SEQUENCE LISTING (47 sequences)

SEQ ID NO: 1 - protein, 10 aa, Bos sp.
YLGYLEQLLR

SEQ ID NO: 2 - protein, 14 aa, Bos sp.
  MISC_FEATURE (10)..(10): X is phosphoserine
VPQLEIVPNXAEER

SEQ ID NO: 3 - protein, 12 aa, Bos sp.
FFVAPFPEVFGK

SEQ ID NO: 4 - protein, 16 aa, Bos sp.
IGVNQELAYFYPELFR

SEQ ID NO: 5 - protein, 19 aa, Bos sp.
EPMIGVNQELAYFYPELFR

SEQ ID NO: 6 - protein, 15 aa, Bos sp.
HQGLPQEVLNENLLR

SEQ ID NO: 7 - protein, 14 aa, Bos sp.
  MISC_FEATURE (10)..(10): X is C8F17-labeled residue
VPQLEIVPNXAEER

SEQ ID NO: 8 - skipped

SEQ ID NO: 9 - protein, 16 aa, Bos sp.
  MISC_FEATURE (12)..(12): X is C8F17-labeled residue
YKVPQLEIVPNXAEER

SEQ ID NO: 10 - protein, 14 aa, Bos sp.
  MISC_FEATURE (10)..(10): X is C4F9-labeled residue
VPQLEIVPNXAEER

SEQ ID NO: 11 - protein, 16 aa, Bos sp.
  MISC_FEATURE (4)..(4): X is C4F9-labeled residue
  MISC_FEATURE (6)..(6): X is C4F9-labeled residue
DIGXEXTEDQAMEDIK

SEQ ID NO: 12 - protein, 12 aa, Bos sp.
  MISC_FEATURE (6)..(6): X is C4F9-labeled residue
TVDMEXTEVFTK

SEQ ID NO: 13 - protein, 10 aa, Artificial
  synthetic peptide
  MISC_FEATURE (1)..(1): X is pyroglutamate
  MISC_FEATURE (3)..(3): X is C8F17-labeled residue
XLXSGVSEIR

SEQ ID NO: 14 - protein, 10 aa, Artificial
  synthetic peptide
  MISC_FEATURE (3)..(3): X is C8F17-labeled residue
QLXSGVSEIR

SEQ ID NO: 15 - protein, 20 aa, Gallus gallus
  MISC_FEATURE (5)..(5): X is phosphoserine
EVVGXAEAGVDAASVSEEFR

SEQ ID NO: 16 - protein, 20 aa, Gallus gallus
  MISC_FEATURE (5)..(5): X is C8F17-labeled residue
EVVGXAEAGVDAASVSEEFR

SEQ ID NO: 17 - protein, 23 aa, Gallus gallus
  MISC_FEATURE (7)..(7): X is C8F17-labeled residue
  MISC_FEATURE (12)..(12): X is cysteic acid
LPGFGDXIEAQXGTSVNVHSSLR

SEQ ID NO: 18 - protein, 26 aa, Gallus gallus
  MISC_FEATURE (10)..(10): X is C8F17-labeled residue
  MISC_FEATURE (15)..(15): X is cysteic acid
FDKLPGFGDXIEAQXGTSVNVHSSLR

SEQ ID NO: 19 - protein, 12 aa, Unknown
  unspecificied, O-GlcNAc peptide
  MISC_FEATURE (6)..(6): X is C8F17-labeled residue
PSVPVXGSAPGR

SEQ ID NO: 20 - protein, 13 aa, Unknown
  unspecificied, O-GlcNAc peptide
  MISC_FEATURE (8)..(8): X is C8F17-labeled residue
PGGSTPVXSANMM

SEQ ID NO: 21 - protein, 13 aa, Unknown
  unspecificied, O-GlcNAc peptide
  MISC_FEATURE (8)..(8): X is C8F17-labeled residue
  MISC_FEATURE (12)..(13): X is oxidized methionine
PGGSTPVXSANXX

SEQ ID NO: 22 - protein, 13 aa, Unknown
  unspecificied, O-GlcNAc peptide
  MISC_FEATURE (8)..(8): X is C8F17-labeled residue
  MISC_FEATURE (12)..(12): X is oxidized methionine
PGGSTPVXSANXM

SEQ ID NO: 23 - protein, 13 aa, Unknown
  unspecificied, O-GlcNAc peptide
  MISC_FEATURE (8)..(8): X is C8F17-labeled residue
  MISC_FEATURE (13)..(13): X is oxidized methionine
PGGSTPVXSANMX

SEQ ID NO: 24 - protein, 11 aa, Unknown
  unspecificied, O-GlcNAc peptide
  MISC_FEATURE (6)..(6): X is C8F17-labeled residue
PSVPVXSAPGR

SEQ ID NO: 25 - protein, 12 aa, Unknown
  unspecificied, O-GlcNAc peptide
  MISC_FEATURE (6)..(6): X is serine-O-GlcNAc
PSVPVXGSAPGR

SEQ ID NO: 26 - protein, 6 aa, Bos sp.
  MISC_FEATURE (1)..(1): X is modified cysteine residue
XASIQK

SEQ ID NO: 27 - protein, 8 aa, Bos sp.
  MISC_FEATURE (3)..(3): X is modified cysteine residue
QNXDQFEK

SEQ ID NO: 28 - protein, 12 aa, Bos sp.
  MISC_FEATURE (3)..(3): X is modified cysteine residue
YIXDNQDTISSK

SEQ ID NO: 29 - skipped

SEQ ID NO: 30 - protein, 14 aa, Bos sp.
  MISC_FEATURE (3)..(3): X is modified cysteine residue
MPXTEDYLSLILNR

SEQ ID NO: 31 - protein, 14 aa, Bos sp.
  MISC_FEATURE (1)..(1): X is oxidized methionine
  MISC_FEATURE (3)..(3): X is modified cysteine residue
XPXTEDYLSLILNR

SEQ ID NO: 32 - protein, 16 aa, Bos sp.
  MISC_FEATURE (3)..(3): X is modified cysteine residue
RPXFSALTPDETYVPK

SEQ ID NO: 33 - protein, 13 aa, Bos sp.
  MISC_FEATURE (6)..(6): X is cysteine residue modified by reaction with tridecafluorooctyl acrylate
DDPHAXYSTVFDK

SEQ ID NO: 34 - protein, 11 aa, Bos sp.
  MISC_FEATURE (3)..(3): X is cysteine residue modified by reaction with tridecafluorooctyl acrylate
YIXDNQTISSK

SEQ ID NO: 35 - protein, 7 aa, Bos sp.
  MISC_FEATURE (3)..(3): X is fluorous tagged cysteine residue
GAXLLPK

SEQ ID NO: 36 - protein, 12 aa, Unknown
  unspecified, polyubiquitin
  MISC_FEATURE (7)..(7): X is lysine residue with a branch containing two glycine residues
LIFAGQXLEDGR

SEQ ID NO: 37 - protein, 12 aa, Artificial
  synthetic peptide
LIFAGKQLEDGR

SEQ ID NO: 38 - protein, 14 aa, Bos sp.
  MISC_FEATURE (3)..(3): X is carbamidomethylated cysteine
MPXTEDYLSLILNR

SEQ ID NO: 39 - protein, 16 aa, Bos sp.
RHPYFYAPELLYYANK

SEQ ID NO: 40 - protein, 12 aa, Bos sp.
  MISC_FEATURE (11)..(11): X is carbamidomethylated cysteine
SLHTLFGDELXK

SEQ ID NO: 41 - protein, 12 aa, Bos sp.
TVMENFVAFVDK

SEQ ID NO: 42 - protein, 15 aa, Bos sp.
KVPQVSTPTLVEVSR

SEQ ID NO: 43 - protein, 13 aa, Bos sp.
LGEYGFQNALIVR

SEQ ID NO: 44 - protein, 13 aa, Bos sp.
DAFLGSFLYEYSR

SEQ ID NO: 45 - protein, 14 aa, Bos sp.
  MISC_FEATURE (3)..(3): X is cysteine residue modified by reaction with tridecafluorooctyl acrylate
MPXTEDYLSLILNR

SEQ ID NO: 46 - protein, 11 aa, Bos sp.
  MISC_FEATURE (3)..(3): X is cysteine residue modified by reaction with tridecafluorooctyl acrylate
YIXDNQTISSK

SEQ ID NO: 47 - protein, 11 aa, Bos sp.
  MISC_FEATURE (3)..(3): X is carbamidomethylated cysteine
YIXDNQTISSK

(19) United States
(12) Patent Application Publication
(10) Pub. No.: US 2006/0263886 A1
(43) Pub. Date: Nov. 23, 2006

(54) Fluorous labeling for selective processing of biologically-derived samples

(76) Inventors: Eric Peters, Carlsbad, CA (US); Scott M. Brittain, San Diego, CA (US); Ansgar Brock, San Diego, CA (US); Scott B. Ficarro, San Diego, CA (US); Arthur R. Salomon, Providence, RI (US)

Correspondence Address: QUINE INTELLECTUAL PROPERTY LAW GROUP, P.C., P O BOX 458, ALAMEDA, CA, 94501, US

(21) Appl. No.: 10/544,609

(22) PCT Filed: Nov. 12, 2004
(86) PCT No.: PCT/US04/37821

§ 371 Date: Aug. 4, 2005

Related U.S. Application Data
(60) Provisional application No. 60/520,736, filed on Nov. 14, 2003.
(60) Provisional application No. 60/612,345, filed on Sep. 22, 2004.

(51) Int. Cl. G01N 33/00

(52) U.S. Cl. 436/56

(57) ABSTRACT

This invention provides fluorous-based methods and compositions for preparation, separation and analysis of complex biologically-derived samples, such as proteomic and metabolomic samples.

### CROSS-REFERENCE TO RELATED APPLICATIONS

This application is related to U.S. provisional patent applications 60/520,736 filed Nov. 14, 2003 and 60/612,345 filed Sep. 22, 2004. The present application claims priority to, and benefit of, these applications, pursuant to 35 U.S.C. §119(e) and any other applicable statute or rule.

### FIELD OF THE INVENTION

The present invention relates to fluorous-based methods for analysis of complex samples such as proteomics and metabolomics samples, and related fluorous compositions.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Proteomics is defined as “the qualitative and quantitative comparison of proteomes (i.e., the protein complement to a genome) under different conditions to further unravel biological processes” (see, for example, the proteomics_def.html at us.expasy.org). Thus, proteomics studies involve the examination of how proteins interact with each other, with their environment, and with other molecules. In a similar manner, metabolomics is the examination and analysis of the small molecule components/inventory of a cell (or multicellular construct, such as a tissue or organism), including, but not limited to, nutrients, vitamins, antioxidants and other redox componentry, various molecules involved in signal transduction and regulation (e.g., nucleotides, hormones, neurotransmitters, and the like), byproducts of metabolism, waste products, non-endogenous components (e.g., pharmaceutical and their derivatives), and the like. The information generated from the profiling of cellular protein and/or metabolite constituents can be used for a number of purposes, many of which focus on the development of an understanding of the underlying characteristics of disease and wellness.

The introduction of biological mass spectrometry (MS) in the early 1990's and its rapid subsequent development have greatly increased researchers' abilities to characterize cellular compositions and the processes in which they are involved (see, for example, Karas and Hillenkamp (1988) Anal. Chem.60:2299-2301; and Fenn et al. (1989) Science 246:64-71. However, the thorough analysis of all of the proteins expressed by an organism at a given time (i.e., the proteome), or the status of the myriad metabolic intermediates, signal transducers, and other small molecules present in a cell at a given point in time (the metabolome), remains elusive. Processes such as RNA processing, proteolytic activation, and hundreds of (often sub-stoichiometric) post-translational modifications (PTMs) can result in the production of numerous proteins of unique structure and function from a limited number of genes; furthermore, numerous biochemical pathways can be involved in the generation and processing of various cellular metabolites. Additionally, there is an issue of detection sensitivity: for example, complex living organisms exhibit extreme dynamic ranges in protein expression levels, ranging from estimated values of 10⁴ in yeast to 10⁹ to 10¹² in plasma (Futcher et al. (1999) Mol. Cell Biol. 19:7357-7368; Corthals et al. (2000) Electrophoresis 21:1104-1115) Due to the extreme complexity thus inherent in biological samples, proteomics and metabolomics studies effectively focus instead on only a subset of the overall protein or metabolite complement.

Numerous methodologies for sample simplification have actively been employed. For example, the initial fractionation of protein samples based on their physical properties such as differing solubility (Nouwens et al. (2000) Electrophoresis 21:3797-3809; Taylor et al. (2000) Electrophoresis 21:3441-3459), isoelectric point (Herbert and Righetti (2000) Electrophoresis 21:3639-3648), or subcellular location (Taylor et al. (2003) Trends Biotechnol. 21:82-88) have been described. Similarly, fractionation schemes based on specific chemical functionalities exhibited by chemical components in a complex sample have also been described, and generally fall into two classes. In some approaches, the entity that directly interacts with a specific chemical functionality also effects the direct enrichment/isolation of the sample components containing this functionality from the remainder of the sample. Embodiments of this fractionation approach include the immobilized metal affinity chromatography (IMAC) enrichment of phosphorylated species (Ficarro et al.(2002) Nature Biotechnol. 20:301-305); the antibody-based enrichment of numerous functionalities (Pandey et al. (2000) Proc. Natl. Acad. Sci. USA 97:179-184; Nikov et al. (2003) Anal. Biochem. 320:214-222); or the enrichment of various glycosylated species using corresponding lectins (Geng et al. (2001) J. Chromatogr. B Biomed. Sci. Appl., 752:293-306). Although highly effective, these approaches require the development of individual enrichment and isolation reagents for each specific functionality.

Alternatively, samples can be chemically altered to assist in the analysis procedure. For example, analysis of N-terminal peptides in a proteolyzed sample can be approached by altering the hydrophobicity of the internal peptides using the reagent 2,4,6-trinitrobenzenesulfonic acid (as described, e.g., in Gevaert et al. “Exploring proteomes and analyzing protein processing by mass spectrometric identification of sorted N-terminal peptides” (2003) J. Nat. Biotechnol. 21:566-569). In a similar manner, 2,4-dinitrofluorobenzene can be used to tag N-termini of hydrolyzed, lysine-protected proteins for the purpose of identifying cross-linked peptides (see, for example, Chen et al. (1999) “Protein cross-links: universal isolation and characterization by isotopic derivatization and electrospray ionization mass spectrometry” Anal. Biochem. 273:192-203). In another approach, a series of dual-functionality reagents are employed, which possess different chemically reactive moieties coupled to a selected affinity moiety, enabling the facile enrichment of specific sample fractions using a common isolation (e.g., affinity-based) methodology. Using this approach, the enrichment of fractions containing particular amino acids (Gygi et al. (1999) Nature Biotechnol.17:994-999), post-translational modifications (Goshe et al. (2001) Anal. Chem. 73:2578-2586), chemical cross-links (Trester-Zedlitz et al. (2003) J. Am. Chem. Soc. 125:2416-2425), or specific enzymatic activities (Campbell and Szardenings (2003) Curr. Opin. Chem. Biol. 7:296-303), has been described. In the overwhelming majority of the proteomics cases, classic biochemical affinity pairs such as biotin-streptavidin are used to effect the isolation of the labeled species. Although effective, the custom reagents employed are relatively expensive, and subject to all the typical limitations in the use of biologically-derived samples.

Additional isolation technologies based upon bead-bound chemical functionalities are known in the art. These include reactions specific for particular chemical functionalities, component elements such as amino acid residues, as well as particular post-translational modifications. After removal of non-bound species, the peptides captured on the solid phase are selectively recovered using specialized release mechanisms chemically designed into the capture reagent. Examples of cleavage processes described in the art include photochemical cleavage (Zhou et al. (2002) Nat. Biotechnol. 20:512-515; Qian et al. (2003) Anal. Chem. 75:5441-5450), acid labile cleavage (Qiu et al. (2002) Anal. Chem. 74:4969-4979), or chemical reagent induced cleavage (Wang et al. (2002) J. Chromatogr. A 949:153-162; Shen et al. (2003) Mol. Cell. Proteomics 2:315-324). Although highly effective, these approaches require the development of individual solid phase reagents for each specific functionality.

Since the introduction of fluorous biphasic catalysis techniques (Horvath and Rabai (1994) Science 266:72-75), the field of fluorous chemistry has expanded rapidly. The term “fluorous” was coined to represent highly fluorinated (or perfluorinated) species in a way analogous to how “aqueous” represents water-based systems. Its original application was the ready separation of reaction products from catalysts based on liquid-liquid partitioning. Specifically, a metal complex bearing one or more highly fluorinated ligands is dissolved in a fluorous solvent, and mixed with reactants dissolved in an organic solvent. Immiscible at room temperature, the two phases become miscible upon heating, enabling the reaction to occur under homogeneous conditions. Upon cooling, phase separation reoccurs, and, under ideal conditions, the organic phase contains only the reaction products, while the fluorous phase contains the catalyst, which can then easily be removed and reused. This concept was quickly extended to fluorous synthesis methodologies in which the reaction substrate itself is made fluorous rather than the catalyst or reagents (Studer et al. (1997) Science 275:823-826).

Although successful, these liquid-liquid extraction methodologies require the use of compounds with extremely high fluorine content. For example, the presence of thirty nine fluorine molecules is required to effectively render “fluorous” small organic molecules with molecular weight less than 150 Daltons (Curran Synlett. 2001 pages 1488-1496). By contrast, replacing the liquid fluorous phase with a solid fluorous phase dramatically expands the practicality of fluorous methodologies. For example, fluorous reversed-phase silica gel has been used to effect fluorous solid phase extraction of appropriated labeled excess reagents or products (see, for example, Zhang et al. (2002) Tetrahedron 58:3871-3875; Markowicz and Dembinski (2002) Org. Lett. 4:3785-3787. Alternatively, fluorous chromatography has been employed to separate members of a solution-phase combinatorial library based primarily of the fluorine content of tags introduced during the reaction sequence (Zhang et al. (2002) J. Am. Chem. Soc. 124:10443-10450). In addition to a variety of classical organic syntheses (see, for example, Dobbs and Kimberley (2002) Journal of Fluorine Chemistry 118:3-17), fluorous methodologies have also recently been applied to the synthesis of small peptides and oligosaccharides (Palmacci et al. (2001) Angew. Chem. Int. Ed. 40:44334437; Filippov et al. (2002) Tetrahedron Letters 43:7809-7812; Miura et al. (2003) Angew. Chem. Int. Ed. 42:2047-2051; and Mizuno et al. (2003) Chem. Commun. (Camb.) 972-973). Fluorous methodologies have also been employed in the kinetic resolution of racemic carboxylic acids and alcohols using lipases that maintain their catalytic activity in dry hydrophobic solvents (Beier and O'Hagan (2002) Chem. Commun. (Camb.) 1680-1681).

Accordingly, the present invention meets a need in the art by providing novel compositions and methods for fluorous proteomics and metabolomics studies, e.g., the analysis of proteomics or metabolomics samples using fluorous methodologies. The methods and. compositions of the present invention can be used to specifically label and manipulate highly complex mixtures of biologically-derived samples in protic solvents. Despite the fact that the labeled species bear fluorous tags that are often considerably smaller than their original molecular mass, the tagged species can still easily be separated from untagged species, and in some embodiments of the invention, from species carrying different fluorous tags. These and other advantages of the present invention will be apparent upon complete review of the following disclosure.

### SUMMARY OF THE INVENTION

The present invention provides fluorous-based methods and compositions for preparation, separation and analysis of complex biologically-derived samples, such as proteomic and metabolomic samples.

In one aspect, the present invention provides methods for preparing one or more compounds in a biologically-derived sample for analysis. The methods include the steps of a) providing a fluorous labeling reagent comprising a chemically-reactive functional group coupled to a fluorous moiety comprising five or more fluorine atoms; and b) coupling the fluorous labeling reagent to one or more member compounds in the biologically-derived sample, via the chemically-reactive functional group, to produce fluorous labeled sample members, thereby preparing the biologically-derived sample for analysis. The biologically-derived sample can be, for example, a proteomics sample or a metabolomics sample; exemplary sample sources include, but are not limited to, cell lysates, cell secretions, tissue samples, bodily fluids such as blood, urine, or saliva, and the like. Optionally, the biologically-derived sample is prefractionated (e.g., by gel electrophoresis or column chromatography).

Optionally, the methods of the present invention further include the step of separating the fluorous labeled sample members from unmodified members using a separating composition having an affinity for the fluorous labeling reagent. For example, in some embodiments, the fluorous labeled sample members are “batch eluted” via a solid phase extraction step. In alternate embodiments, the fluorous labeled sample members are separated from the unmodified members by performing fluorous column chromatography using a fluorous affinity matrix, such as fluorous silica gel, and collecting a column effluent of interest (e.g., either the unbound species or the fluorous labeled species, or both). Optionally, eluting the bound fraction can also include separating singly-labeled sample members from multiply-labeled sample members. In another embodiment, the fluorous affinity matrix is associated with a 2-dimensional surface, such as the surface of a MALDI plate or a DIOS plate, such that separating the fluorous labeled sample members involves applying the sample to the surface containing the affinity matrix and washing away unbound sample members.

Optionally, the methods of the present invention further include the step of analyzing the biologically-derived sample, e.g., by performing mass spectrometry on a separated (labeled or unlabeled) fraction of the biologically-derived sample. In some embodiments, the analyzing step includes comparing MS data for the separated fraction with MS data for an unreacted aliquot of the biologically-derived sample.

Optionally, the fluorous labeling reagent is a composition that is stable during a selected analysis procedure, e.g., one that is minimally fragmented under standard ionization and/or fragmentation conditions for mass spectroscopy.

A variety of chemically-reactive functional groups can be incorporated into the fluorous labeling reagents of the present invention, including, but not limited-to, a maleimide, a halogen β-ketone, a disulfide exchange reagent, a phenylglyoxal, an anhydride, an acrylate, an azide, a thiol, a dihydroxy borane or boronic acid, an N-hydroxysuccinimide ester or sulfo N-hydroxysuccinimide ester, a dialkyl pyrocarbonate, a Michael donor, an aminooxy compound, or a hydrazine-containing compound. In some embodiments, the chemically-reactive functional group is chosen such that the fluorous labeling reagent is an amino acid conjugation agent.

The fluorous moiety of the fluorous labeling reagent typically comprises five or more fluorine atoms. In many embodiments, this fluorous moiety is a fluoroalkyl group having the formula CF₃(CF₂)ₙ, wherein n is an integer between 2 and 10, or optionally between 3 and 7. In some embodiments, the fluorous moiety is a branched fluoroalkyl moiety, or a fluoroalkyl structure in which a limited number of the fluorine atoms are replaced with other atoms such as hydrogen, deuterium, or other halogens. Optionally, the fluorous labeling reagents of the present invention include first and second chemically-reactive functional groups coupled to one another via the fluorous moiety (e.g., a fluoroalkyl linker).

Optionally, the fluorous labeling reagent comprises a mixture of reagents. For example, the labeling reagent can include a first member having a first chemically-reactive functional group coupled to a first fluorous moiety, and a second member comprising a second chemically-reactive functional group coupled to a second fluorous moiety. Optionally, the first and second fluorous moieties differ in their affinity for the separating composition.

In addition to targeting naturally-occurring chemical moieties in a select sample, a reactive functionality can be introduced into the biologically-derived sample to facilitate the fluorous labeling. For example, in some embodiments of the present invention, a periodate oxidation can be performed on the biologically-derived sample, to generate sample components having one or more aldehyde groups. This is particularly useful for preparing glycosylated sample members for analysis. One or more sugars on the glycosylated sample members are oxidized to generate one or more aldehyde moieties in the reaction mixture, to which is added a hydrazine-type or aminooxy-type fluorous labeling reagent (e.g., in which the chemically reactive functional group is a hydrazine or aminooxy moiety). The aldehyde moieties react with, e.g., the hydrazine to form a (fluorous) hydrazide product, thereby labeling the glycosylated sample member. Alternatively, reaction of the aldehyde with the aminooxy reagent produces an fluorous labeled oxime product.

In some embodiments, the sample members selected for labeling are phosphorylated amino acid-containing components (e.g., phosphorylated serine residues, phosphorylated threonine residues, and/or phosphorylated tyrosine residues). Phosphorylated serine and threonine members can be labeled by making the reaction mixture basic, performing a β-elimination reaction on the phosphorylated amino acid-containing components, adding the fluorous labeling reagent to the reaction mixture, followed by performing a Michael addition reaction on a product of the β-elimination reaction, thereby coupling a fluorous label from the fluorous labeling reagent at a previous site of phosphorylation. While numerous reagents can be used to interact with the dephosphorylated product of the β-elimination reaction, fluorous-modified thiol reagents such as CF₃(CF₂)₇CH₂CH₂SH are particularly easy to use.

Alternatively, all three phosphorylated amino acid residues can be labeled by performing a carboxylic acid methylation on the sample under acidic conditions, followed by an EDC-mediated coupling of cystamine to the phosphate group, to produce a phosphoramidate species. The cystamine is reduced to form a free thiol, via which the fluorous labeling reagent can be coupled, thereby labeling the phosphorylated sample member. After fluorous solid phase extraction (FSPE) of the labeled species, the method optionally includes the acid release of the fluorous label from the methylated phosphopeptides. This approach provides for the isolation and/or enrichment of phosphotyrosine containing as well as phosphoserine and phosphothreonine containing sample components.

In a further aspect, the present invention provides methods for separating one or more members of a biologically-derived sample. The methods include the steps of a) reacting the biologically-derived sample with at least one fluorous labeling reagent comprising a chemically-reactive functional group coupled to a fluorous moiety comprising five or more fluorine atoms, thereby attaching a fluorous label to one or more sample members to form labeled sample members; and b) separating the fluorous labeled sample members from unmodified sample members using a composition having an affinity for the fluorous label. Optionally, separating the labeled and unmodified sample members can be performed by solid phase (e.g., batch) elution. Alternatively, the separation step can be performed by fluorous column chromatography using, e.g., a fluorous affinity matrix such as fluorous silica gel, and collecting a column eluent. In a further embodiment, the composition having an affinity for the fluorous label is coupled to a surface of a substrate (such as a MALDI or DIOS plate); separating the fluorous labeled sample members from the unmodified sample members can be achieved by applying the biologically-derived sample to the fluorous surface of the substrate and removing the unmodified sample members, e.g., by washing.

The present invention also provides methods for analyzing a complex composition comprising a plurality of biologically-derived components, such as a proteomics sample or metabolomics samples having a plurality of amino acid-containing components (e.g., proteins, proteolytic peptides, and the like). The methods include the steps of a) providing a fluorous labeling reagent comprising a fluorous moiety (having five or more fluorine atoms) coupled to a chemically-reactive functional group; b) modifying one or more members of the complex composition with the fluorous labeling reagent to form a modified composition comprising fluorous labeled components and unlabeled components; c) fractionating or separating the modified composition using a composition having an affinity for the fluorous moiety of the fluorous labeling reagent; and d) performing mass spectrometry a separated sample fraction and generating mass spectral data, thereby analyzing the complex composition.

In a further embodiment, the present invention provides methods for analyzing a biologically-derived sample by a) reacting the biologically-derived sample with a fluorous labeling reagent comprising a chemically-reactive functional group coupled to a fluoroalkyl moiety comprising five or more fluorine atoms, to form a treated sample, thereby incorporating a fluorous label into one or more member components of the biologically-derived sample and forming fluorous modified components; b) analyzing a first portion of the treated proteomics sample by mass spectrometry and generating a first set of mass spectral data; c) analyzing a second portion of the treated proteomics sample by mass spectrometry and generating a second set of mass spectral data, wherein the fluorous modified components of the second portion have been removed by fluorous-based separation techniques using a fluorous affinity matrix prior to analyzing; and d) comparing the first and second sets of mass spectral data and determining one or more mass spectral peaks which are present in the first portion and absent in the second portion, thereby analyzing the biologically-derived sample. Optionally, the data comparison step further includes identifying the mass spectral peaks which are present in the first portion and/or absent in the second portion.

In another aspect, the present invention provides methods for separation of differentially labeled components in a biologically-derived sample, such as a proteomics sample or metabolomics sample, using fluorous-based separation techniques. The methods include the steps of a) providing a biologically-derived sample having a plurality of amino acid-containing components; b) treating the biologically-derived sample with a fluorous labeling reagent and labeling one or more member components, where the fluorous labeling reagent comprising a chemically-reactive functional group coupled to a fluorous moiety having five or more fluorine atoms; c) combining the treated sample with a fluorous affinity matrix; and d) selectively eluting bound single-labeled components separately from bound multiply-labeled components.

The chemically-reactive functional group (which, in some embodiments, is a peptide terminus conjugation agent) element of the fluorous labeling reagent can be a primary amine blocking reagent (e.g., an N-terminal labeling reagent) or a carboxyl blocking reagent (e.g., a C-terminal labeling reagent). Fluorous silica gel can be used to separate the labeled and unlabeled proteins (or protein fragments), which then can be analyzed, e.g., by mass spectrometry.

In embodiments involving the analysis of ubiquitinated components, the sample is often further treated (prior to interaction with the fluorous labeling reagent). Typically, the epsilon-amino groups of any unmodified (i.e., non-ubiquitinated) lysine residues are blocked. Sample members are optionally cleaved (e.g., with trypsin or another proteolytic enzyme), to generate a plurality of proteolytic fragments. This can be performed either prior to or after the lysine 8-amino group blocking step. The N-termini of the peptides are labeled with the fluorous labeling reagent. Due to the presence of the ubiquitin moiety, the pool of proteolytic fragments include a first portion of proteolytic fragments having a single peptide N-terminus, and a second portion of proteolytic fragments having two N-termini (a first peptide-derived N-terminus and a second ubiquitin-derived N-terminus). Both the first and second N-termini of the proteolytic fragments are labeled with the fluorous labeling reagent, to produce a first portion of single-labeled proteolytic fragments and a second portion of multiply-labeled proteolytic fragments.

In a similar manner, the methods of the present invention can also be used to examine intermolecular disulfide bridge-containing components in a proteomics sample. Treating the proteomics sample can optionally include the step of cleaving the disulfide bridge-containing components of the proteomics sample with a proteinase, thereby generating one or more disulfide-linked proteolytic fragments having two N-termini; and labeling both N-termini of disulfide-linked proteolytic fragments.

In a further aspect, the present invention provides methods of separating components of a set of biologically-derived sample, such as series of proteomics or metabolomics samples, using fluorous-based separation techniques. The separation methods include the steps of a) providing a set of biologically-derived samples, wherein each member sample comprises a plurality of components (e.g., amino acid-containing components); b) providing two or more fluorous labeling reagents which differ in the number of fluorine atoms incorporated therein; c) treating a first member of the set of samples with a first fluorous labeling reagent, thereby labeling one or more components of a first sample; d) treating a second member of the set of samples with a second fluorous labeling reagent, thereby labeling one or more components of the second sample; e) combining the first and second samples to form a combined sample; and f) performing a fluorous-based separation technique (such as a fluorous solid phase extraction or fluorous column chromatography) using a fluorous affinity matrix on the combined sample, thereby separating components of the set of biologically-derived samples.

Optionally, additional members of the sample set can also be treated using additional fluorous labeling reagents, which reagents differ from each other and from the first and second fluorous labeling reagents in the number of fluorine atoms incorporated therein. These additional labeled samples are combined with the first and second samples, prior to separation in the fluorous-based separation step.

In some embodiments, the methods are employed to separate non-labeled components from fluorous labeled components. Optionally, the components labeled with the first fluorous labeling reagent can also be separated from the components labeled with the second fluorous labeling reagent. The methods optionally further include the step of analyzing the non-labeled components or the fluorous labeled components (either combined or fractionated) by mass spectrometry.

The present invention also provides novel fluorous labeling reagents, having one or more fluorous moieties coupled to chemically-reactive functional group (e.g., to form a fluorous bioconjugation agent for derivatizing a chemical functionality on a target sample member, for example, an amino acid-associated functional group or a post-translational modification). Typically, the fluorous moieties incorporated into the labeling reagent comprise five or more fluorine atoms, either in a contiguous stretch or clustered into two or more regions of the molecule. In some embodiments, the fluorous labeling reagents have multiple fluorous moieties (e.g., a first fluorous moiety coupled at a first position within the fluorous labeling reagent, and a second fluorous moiety coupled at a second position on the fluorous labeling reagent). In additional embodiments, the fluorous labeling reagents have multiple chemically-reactive functional groups.

Because the compositions of the present invention are often used in conjunction with proteomics and/or metabolomics samples, many embodiments of the fluorous labeling reagents of the present invention are compatible with aqueous reaction conditions. Furthermore, since mass spectrometry is commonly employed in the analysis of such sample, the fluorous labeling reagents optionally are inert under standard ionization and/or fragmentation conditions using in mass spectrometry (for example, the low energy collisions employed in tandem MS).

In some embodiments of the present invention, the fluorous moiety is coupled to the chemically-reactive functional group via a linker region. Typically, the linker region is an alkyl chain between two and twenty carbons in length. In some embodiments, the fluorous portion of the labeling reagent and/or the linker region includes an isotopic label, such as one or more ²H, ¹³C, ¹⁵N or ¹⁸O atoms. Optionally, the linker region (or another part of the bioconjugation agent) includes a releasable element, e.g., to facilitate dissociation of the labeled sample member from the fluorous affinity matrix or other separation composition. For example, chemical moieties sensitive to enzymatic cleavage, chemical cleavage, photolysis, and/or thermal degradation can be used as releasable elements in the compositions and methods of the present invention.

Any of a number of reactive groups can be targeted using the fluorous labeling reagents of the present invention, including, but not limited to, a sulfhydryl group, a thioether group, an amino group, a carboxyl group, a hydroxyl group, an imidazole group, a guanidino group, or an indole moiety. Often, the targeted chemical functional group is associated with an amino acid (e.g., the side chain). In some embodiments, the amino acid-associated functional group is a post-translational modification (PME) element, for example, a phosphate moiety or a saccharide moiety. Also included are chemically-modified PTM elements, as well as a product resulting from removal of a post-translational modification.

Thus, the chemical functionalities that can be employed as chemically-reactive functional groups in the compositions of the present invention include, but are not limited to, a number of chemical species known to react with amino acid-associated reactive groups, such as maleimides, halogen β-ketones, disulfide exchange reagents, phenylglyoxal derivatives, anhydrides, NHS esters and NHS sulfoesters, dialkyl pyrocarbonates, alkyl aminooxy compounds, and hydrazine-containing compounds.

Exemplary fluorous labeling reagents of the present invention include, but are not limited to, 1H,1H,2H,2H-perfluorodecane-1-thiol (1a), 1H,1H,2H,2H-perfluorooctane-1-thiol (1b), 1H,1H,2H,2H-perfluorohexane-1-thiol (1f), N-(3-(perfluorooctyl))propylmaleimide (2a), N-(3-(perfluorohexyl))propylmaleimide (2b), 2-pyridyl-2′-1H,1H,2H,2H-perfluorodecane disulfide (3), (1H,1H,2H,2H-perfluorooctyl)acrylate (4a), (1H,1H,2H,2H-perfluorodecyl)acrylate (4b), N-succinimidyl-2H,2H,3H,3H-perfluoroheptanoate(5aN-sulfosuccinimidyl-2H,2H,3H,3H-perfluoroheptanoate (5b), N-succinimidyl-2H,2H,3H,3H-perfluoroundecanoate (5c), N-sulfosuccinimidyl-2H,2H,3H,3H-perfluoroundecanoate (5d), N-iodoacetyl-3-(perfluorooctyl)propylamine (6a), N-iodoacetyl-3-(perfluorohexyl)propylamine (6c), 3-(perfluorooctyl)glutaric anhydride (7), 4-[3-(perfluorooctyl)propyl-1-oxy]phenyl glyoxyl (8), 2-nitro-4-(N-(3-(perfluorooctyl)propyl)carboxamide)benzenesulfonyl chloride (9), 2H,2H,3H,3H-perfluononanoic acid hydrazide (10), bis(sulfosuccinimidyl)-2H,2H,3H,3H,10H, 10H, 11H, 11H-perfluorododecanedionate (11), sulfosuccinimidyl-12-[(iodoacetyl)amino]2H,2H,3H,3H,10H,10H,11H, 11H, 12H,12H-perfluorododecanoate (12), 1-(1H, 1H, 2H,2H,3H,3H-perfluorononyl)-pyrrole-2,5-dione (13), 2-aminooxy-N-(3,3,4,4,5,5,6,6,7,7,8,8,8-tridecafluoro-octyl)-acetamide (14), 1-azido-(1H, 1H, 2H,2H,3H,3H-perfluorononane (15a), 1-azido-(1H, 1H, 2H,2H,3H,3H-perfluorounde (15b), 2-aminooxy-N-(4,4,5,5,6,6,7,7,7-nonafluoro-heptyl) acetamide, (16a), 2-aminooxy-N-(4,4,5,5,6,6,7,7,8,8,9,9,10,10,11,11,11-heptadecafluoro-undecyl)acetamide (16b), 4,4,5,5,6,6,7,7,7-nonafluoro-heptanoic acid N′-(2-aminooxy-acetyl)hydrazide (16c), 4,4,5,5,6,6,7,7,8,8,9,9,10,10,11,11,11-heptadecafluoro-undecanoic acid N′-(2-aminooxy-acetyl)hydrazide (16d), 1-amino-(1H, 1H, 2H,2H,3H,3H-perfluorononane (17), p-(1H, 1H, 2H,2H-perfluorodecyl)-phenylboronic acid (18), 1-aminooxy-5,5,6,6,7,7,8,8,9,9,10,10,11,11,12,12,12-heptadecafluoro-dodecan-2-one (19), 1-(fluoroethoxyphosphinyl)-3,3,4,4,5,5,6,6,7,7,8,8,9,9,10,10,10-heptadecafluoro-decane (20), phosphoric acid mono-{4-[fluoro-(4,4,5,5,6,6,7,7,8,8,9,9,10,10,11,11,11-heptadecafluoro-undecylcarbamoyl)-methyl]-phenyl}ester (21), and 6-[3-(3,3,4,4,5,5,6,6,6-nonafluoro-hexyldisulfanyl)-propionylamino]-hexanoic acid 2,5-dioxo-pyrrolidin-1-yl ester (25).

Optionally, the fluorous labeling reagents of the present invention can further include an additional chemically-reactive functional group for derivatizing an additional amino acid-associated functional group. In embodiments having multiple chemically-reactive functional groups, the bioconjugation elements need not be of the same structure or have an affinity for the same target or type of amino acid residue (i.e., a two-pronged fluorous labeling reagent can be used to couple disparate chemical entities).

The present invention also provides methods for fractionating fluorous and non-fluorous components of a fluorous labeled sample directly on a 2-dimensional surface. The methods include the steps of providing a composition having an affinity for a fluorous label, which composition is coupled to a first portion of the surface of the substrate; loading a fluorous labeled sample comprising fluorous components and nonfluorous components onto the surface of the substrate and associating the fluorous components of the sample with the composition having an affinity for a fluorous label; and removing the nonfluorous components, thereby fractionating a fluorous labeled sample on the substrate surface. In an exemplary embodiment, removing the nonfluorous components is performed by washing the surface of the substrate, thus separating the fluorous components from the nonfluorous components. In some embodiments, the substrate is a MS substrate, such as a MALDI plate or a DIOS plate, such that washing the substrate surface leaves the fluorous components in place for further analysis by mass spectrometry. Optionally, the first portion of the substrate surface includes a majority (or all) of the surface. Alternatively, the first portion of the surface can comprise one or more specified locations on the substrate surface (e.g., positions that correlate to arrayed positions from which the samples are obtained, such as microtiter wells).

As a further aspect, the present invention also provides sets of fluorous labeling reagents, which can be used, for example, for differential quantification of a proteomics sample. Typically, a set of fluorous labeling reagents includes two or more fluorous labeling reagents of the present invention, wherein the reagents are differentially labeled with one or more stable isotopes (e.g., deuterium or ¹³C).

These and other objects and features of the invention will become more fully apparent when the following detailed description is read in conjunction with the accompanying figures.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a fluorous moiety” includes a combination of two or more fluorous moieties; reference to “biologically-derived sample” includes mixtures of samples, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

As used herein, the term “biologically-derived sample” refers to a plurality of components isolated or otherwise obtained from a biological source, such as a eukaryotic or prokaryotic cell, and includes cellular lysates as well as bodily fluids (e.g., blood, urine, etc.) or other cellular secretions (e.g., culture media which has been exposed to cells or tissues) from an organism. Exemplary embodiments include, but are not limited to, proteomic samples, metabolomics samples, and glycomics samples. A proteomics sample typically comprises a set of protein compositions derived from a corresponding cellular genome. The proteomics sample can be a complete set of proteins that the cell is capable of generating, or a subset of proteins (e.g., selected based upon an expression pattern or a fractionation technique). In a similar manner, a metabolomics samples comprises a corresponding population of small molecule components present in a cell or other biologically-derived sample, while a glycomics sample contains various carbohydrate-based components (e.g., simple sugars, complex carbohydrates, proteoglycans, glycoproteins, glycolipids, and the like).

The term “fluorous” as used herein refers to fluorine-containing chemical moieties, and includes both partially and fully fluorous (e.g., perfluoro) compositions.

The term “bioconjugation agent” is used herein to refer to a chemical moiety comprising a chemically-reactive functional group for use in the fluorous labeling reagents of the present invention. For example, a “bioconjugation agent for derivatizing an amino acid-associated functional group” (also termed an “amino acid conjugation agent”) refers to reactive agents that are capable of reversible or irreversibly interacting with a functional group on an amino acid. The reactive functional group can be either a portion of the amino acid itself, or a functionality associated the amino acid, such as a post-translational modification or chemical modification (e.g. β-elimination reaction).

The term “amino acid-containing components” includes any of a number of components present in a biologically-derived sample and having either natural or unnatural amino acids (e.g., amino acid analogs, mimetics, and the like) linked by peptide bonds. Amino acid-containing components of the present invention include, but are not limited to, peptides, oligopeptides, polypeptides, proteins, protein complexes, and the like.

A “protic solvent” is a solvent having a reactive proton, while an “aprotic solvent” is a solvent that does not have a reactive proton.

The terms “aqueous compatible” and “aqueous tolerant,” as used herein with respect to fluorous labeling reagents, refer to compositions which are not rapidly consumed or otherwise deactivated by the solvent system (e.g., prior to having the opportunity to interact with the sample). Typically, the fluorous labeling reagents are employed under reaction conditions in which the concentration of protic solvent(s) (e.g., H₂O) is substantially greater than the concentration of target species to be labeled (e.g., the members of the biologically-derived sample). When freshly prepared, preferably in the presence of the sample to be labeled, the majority (e.g., greater than 50%) of the chemically reactive functional groups in an aqueous compatible fluorous labeling reagent are capable of interacting with the members of the biologically-derived sample (i.e., reaction kinetics favor interaction of the fluorous labeling reagents with the biologically-derived sample members as compared to solvent molecules.) In other words, the water molecules do not out-compete the targeted species for reaction with the fluorous reagent.

Aqueous reaction conditions and/or aqueous solvent systems include, but are not limited to, solvent systems comprising as little as at least 10%, or optionally 25%, 50% or more protic solvents (e.g., water, methanol, etc.) by volume. Optionally, the aqueous solvent systems comprises 75% or more protic solvents by volume, or 90% or more protic solvents, or in some embodiments 100% protic solvents.

The term “fluorous-based separation techniques” as used herein includes, but is not limited to, both liquid and solid phase extraction techniques (e.g., bulk extractions) as well as fluorous chromatography (e.g., the eluting of separate fractions).

### BRIEF DESCRIPTION OF THE DRAWINGS

FIG. 1 provides a schematic representation of fluorous solid phase extraction FSPE) methodology for the isolation of fluorous tagged peptides from a complex peptide mixture.

FIGS. 2A and 2B depict exemplary two step reaction schemes involving β-elimination under basic condition followed by Michael addition of a fluorous thiol, for the selective reaction and subsequent isolation of phosphoserine (pS)/phosphothreonine (pT)-modified or O-GlcNAc-modified (S(OGlcNAc) and T(OGlcNAc)) peptides.

FIG. 3A depicts two exemplary reactions by which the ε-amine functionality of lysine can be blocked by conversion to homoarginine or an imidazoyl moiety. FIG. 3B provides an exemplary reaction scheme depicting conversion of the ε-amino group of lysine to homoarginine (using O-methylisourea), followed by fluorous labeling of the peptide N-terminal amino group. FIG. 3C schematically depicts a procedure for separating linear from branched peptides via a similar fluorous fractionation scheme (in which R_{f} is the fluorous moiety portion of the labeling reagent, e.g., C₄F₉).

FIG. 4A provides an exemplary reaction scheme involving the Michael addition of thiol units to fluorous Michael acceptors for the selective reaction and subsequent isolation of cysteine-containing peptides. FIG. 4B provides an exemplary reaction scheme depicting reaction of thiols with an iodoacetamide-type fluorous labeling reagent.

FIG. 5 depicts an alternative reaction scheme for isolation and/or enrichment of phosphopeptides, including phosphotyrosine species, in which the fluorous label can be removed after isolation/enrichment of the tagged species.

FIG. 6 provides MAILDI spectra of samples generated from a tryptic digest of α-casein prior to (FIG. 6A) and after β-elimination and subsequent reaction with 1H,1H,2H,2H-perfluorodecane-1-thiol (FIG. 6B), as well as the resulting FSPE fractionated peptides (FIG. 6C=non-retained portion, FIG. 6D=retained and eluted fraction; u¹=modified residue).

FIG. 7 depicts a tandem MS spectrum of +2 charge state of peptide VPQLEIVPNu¹AEER (SEQ ID NO:7) residing in retained and subsequently eluted fraction after FSPE as described in FIG. 6 (u¹=residue modified with 1H,1H,2H,2H-perfluorodecane-1-thiol).

FIG. 8 depicts a tandem MS spectrum of +2 charge state of a similar peptide VPQLEIVPNu³AEER (SEQ ID NO:10) labeled in a manner similar to that depicted in FIG. 7 but using 1H,1H,2H,2H-perfluorohexane-1-thiol as the fluorous labeling reagent (u³=modified residue).

FIG. 9 depicts a tandem MS spectrum of +2 charge state of peptide DIGu³Eu³TEDQAMEDIK (SEQ ID NO:11) fluorous labeled with 1H,1H,2H,2H-perfluorohexane-1-thiol and subsequently eluted during FSPE (u³=modified residue).

FIG. 10 depicts a tandem MS spectrum of +2 charge state of peptide TVDMEu³TEVFTK (SEQ ID NO:12) labeled using 1H,1H,2H,2H-perfluorohexane-1-thiol and subsequently eluted during FSPE (u³=modified residue).

FIG. 11A provides the MALDI spectrum of a mixture of two synthetic phosphoserine (pS)-containing peptides (qLu¹SGVSEIR, SEQ ID NO:13 and QLu¹SGVSEIR, SEQ ID NO:14) that were first subjected to β-elimination and subsequent reaction with 1H,1H,2H,2H-perfluorodecane-1-thiol, and then spiked into a tryptic digest of non-modified peptides. FIG. 11B depicts data for the non-retained portion upon FSPE, while FIG. 11C depicts data for the retained and subsequently eluted fraction after FSPE, showing recovery of spiked peptides.

FIG. 12 provides MALDI spectra of a tryptic digest of ovalbumin before (FIG. 12A) and after B-elimination and subsequent reaction with 1H,1H,2H,2H-Perfluorodecane-1-thiol (FIG. 12B), as well as upon FSPE (FIG. 12C=non-retained portion, FIG. 12D=fraction retained and subsequently eluted).

FIG. 13 depicts a tandem MS spectrum of +2 charge state of peptide EVVGu¹AEAGVDAASVSEEFR (SEQ ID NO:16) generated as described in FIG. 12 and residing in the retained and subsequently eluted fraction after FSPE (u¹=modified residue).

FIG. 14 depicts a tandem MS spectrum of +3 charge state of peptide LPGFGDu¹IEAQcGTSVNVHSSLR (SEQ ID NO:17) residing in the retained and subsequently eluted fraction after FSPE as described in FIG. 12 (u¹=modified residue, c=cysteic acid).

FIG. 15 depicts a tandem MS spectrum of +3 charge state of peptide FDKLPGFGDu¹IEAQcGTSVNVHSSLR (SEQ ID NO:18) residing in retained and subsequently eluted fraction after FSPE (u¹=modified residue, c=cysteic acid).

FIG. 16 provides MAIII spectra of a tryptic digest of non-modified peptides spiked with a mixture of two synthetic O-GlcNAc-containing peptides (FIG. 16A) and the peptides retained and subsequently eluted fraction after FSPE (FIG. 16B), demonstrating recovery of the spiked peptides after first subjecting the spiked tryptic digest to β-elimination and subsequent reaction with 1H,1H,2H,2H-perfluorodecane-1-thiol.

FIG. 17 depicts a tandem MS spectrum of +2 charge state of peptide PSVPVuGSAPGR (SEQ ID NO:19) depicted in FIG. 16B and residing in the retained and subsequently eluted fraction after FSPE (u=modified residue).

FIG. 18 depicts a tandem MS spectrum of +2 charge state of peptide PSVPVS_{G}GSAPGR (SEQ ID NO:25) before β-elimination and subsequent reaction with 1H,1H,2H,2H-perfluorodecane-1-thiol (S_{G}=serine-O-glcNAc).

FIG. 19A provides a MALDI spectrum of a mixture of two synthetic pS-containing peptides that were first subjected to -elimination and subsequent reaction with 1H,1H,2H,2H-perfluorodecane-1-thiol, and then spiked into a tryptic digest of the entire soluble protein fraction from pervanadate-treated Jurkat cells. FIG. 19B provides the MALDI spectrum for the non-retained portion upon FSPE, while FIG. 19C depicts the retained and subsequently eluted fraction after FSPE, showing recovery of the spiked peptides (SEQ ID NOS: 13 and 14, u¹=modified residue).

FIG. 20 provides MALDI spectra generated for a tryptic digest of bovine serum albumin after reduction with TCEP and reaction of the cysteine residues with tridecafluorooctyl acrylate (FIG. 20A), the non-retained portion upon FSPE (FIG. 20B), and the retained and subsequently eluted fraction after FSPE (FIG. 20C).

FIG. 21 depicts a tandem MS spectrum of +2 charge state of peptide DDPHAc^{†}YSTVFDK (SEQ ]D NO:33) residing in retained and subsequently eluted fraction after FSPE from FIG. 20 (c^{\}=modified residue).

FIG. 22 depicts a tandem MS spectrum of +2 charge state of peptide YIc^{†}DNQTISSK (SEQ ID NO:34) residing in retained and subsequently eluted fraction after FSPE from FIG. 20 (c^{†}=modified residue).

FIG. 23 provides MALDI spectra generated for a tryptic digest of bovine serum albumin after reduction with TCEP and reaction with N-[(3-perfluorooctyl)-propyl]iodoacetamide (FIG. 23A), and the retained and subsequently eluted fraction after FSPE (FIG. 23B, (C*=peptides containing modified cysteine residues(s)).

FIG. 24 depicts a tandem MS of 2+charge state of peptide GAC*LLPK (SEQ ID NO:35) residing in the retained and subsequently eluted fraction after FSPE as shown in FIG. 23B (C*=modified residue, C*₁ =immonium ion of the modified cysteine residue; C*L and C*LL are internal fragments).

FIG. 25 provides MALDI spectra of tryptic digest of polyubiquitin before (FIG. 25A) and after (FIG. 25B) reaction with O-methylisourea to selectively block lysine residues. After reaction with N-hydroxysuccinimidyl-2H,2H,3H,3H-perfluoroheptanoate (FIG. 25C, solid circle=fluorous moiety), singly-labeled and doubly labeled species are generated. FSPE is performed under conditions such that species bearing one fluorous tag are not retained, while those bearing two tags are retained and subsequently eluted (FIG. 25D).

FIGS. 26-28 provide LC/MS chromatographic elution profiles of synthetic peptide LUFAGQKLEDGR (SEQ ID NO:37) labeled at the N-terminus with N-hydroxysuccinimidyl-2H,2H,3H,3H-perfluoroheptanoate, as well as four bovine serum albumin tryptic peptides, resolved upon C₈F₁₇ modified silica (FIG. 26), C₆F₁₃ modified silica (FIG. 27), and C₆F₅ pentafluorophenyl modified silica (FIG. 28).

FIG. 29A depicts a tandem MS spectrum of +2 charge state of tryptic peptide MPc^{†}TEDYLSLILNR (SEQ ID NO:45) from, reduced bovine serum albumin, after reaction of cysteine residue with tridecafluorooctyl acrylate (c^{†}=modified residue). FIG. 29B provides the spectrum for the native peptide MPcTEDYLSLILNR (SEQ ID NO:38, in which c=carbamidomethylated cysteine).

FIGS. 30A and 30B depict tandem MS spectra of +2 charge state of peptide YIc^{†}DNQTISSK (SEQ ID NO:46) from reduced bovine serum albumin after reaction of cysteine residue with tridecafluorooctyl acrylate (c^{†}=modified residue), and native peptide YIcDNQTISSK. (SEQ ID NO:47, in which c=carbamidomethylated cysteine).

FIGS. 31 and 32 provide additional exemplary reaction schemes employing fluorous labeling reagents of the present invention.

### DETAILED DESCRIPTION

The present invention provides novel methods for the analysis of complex biological samples, such as proteomics and metabolomics samples, as well as fluorous labeling reagents for use in fluorous applications. The methods and compositions of the present invention enable the analysis of proteomics and/or metabolomics components in a manner highly orthogonal to other such techniques currently employed. The methods of the present invention take advantage of the unique self-associative interactions of fluorous moieties, facilitating the separation of labeled and unlabeled species, as well as enabling multiplexed separations of differentially-labeled species. Furthermore, unlike other labeling reagents described in the art, the fluorous labels provided herein typically are chemically inert and/or stable during processing and analysis.

Characteristics of Fluorous Moieties

The unique selectivity of fluorous-based separation techniques provides an novel approach for the selective isolation of labeled species from a complex mixture, eliminating many of the non-specific interactions characteristic of biological-based affinity methods. Fluorous moieties such as perfluoroalkyl groups tend to associate primarily with “like” or similar compositions (e.g., themselves, or other fluorous containing compositions). This property has been utilized by those skilled in the art for liquid-liquid and liquid-solid extractions during chemical syntheses, as well as in fluorous chromatography. Segregation between fluorous-containing and non-fluorous containing compositions can be achieved independent of the nature (e.g., molecular weight) of the chemical entity attached to the fluorous label(s). Additionally, chemical species having fluorous labels of different chain lengths can be separated from one another, demonstrating retention properties that persist regardless of the nature of the bound species.

Fluorous methodologies have been used in combinatorial syntheses as an alternative to conventional solid and solution phase approaches (see, for example, Zhang et al. (2002) “Solution Phase Preparation of a 560-compound library of individual pure mappine analogues by fluorous mixture synthesis” J. Am. Chem. Soc. 124:10443-10450; as well as U.S. Pat. No. 5,777,121; U.S. Pat. No. 5,859,247; and U.S. Pat. No. 6,156,896 to Curran et al.). Additionally, fluorous species have been used as reagents, scavengers, and catalyst in organic synthesis methodologies (see, for example, Lindsley et al. (2002) Tetrahedron Letters 43:6319-6323; Zhang et al. (2003) Tetrahedron Letters 44:2065-2068; Zhang et al. (2000) J. Org. Chem. 65:8866-8873; and Zhang (2003) Tetrahedron 59:44754489). However, in all cases, these methods have been employed during the targeted synthesis and purification of specific organic molecules, rather than the isolation of a specific subfraction of a more complex, preformed mixture (e.g., such as a cellular extract or other biologically-derived sample). The fluorous species employed in these processes typically have molecular weights greater or similar to the chemical synthesis intermediate to which they are bound and are only used in conjunction with aprotic solvent(s).

The present invention provides fluorous-based methods and compositions that are not limited to incorporation of fluorous moieties into low molecular weight synthetic intermediates in organic reaction mixtures. Rather, the methods and compositions of the present invention can be employed with biological products covering a range of sizes, which products may be present in either organic or aqueous (or other protic) solutions. For example, highly complex mixtures of peptides and/or proteins, such as typically present in a proteomics sample, can be labeled with one or more fluorous labeling reagents of the present invention, which reagent has been selected or designed to react with a specific functionality present in the sample. While the fluorous tags optionally are considerably smaller than the species to be labeled (i.e., the label might not dramatically change the molecular weight of the bound species), the tagged species can still easily be separated from untagged species using, for example, readily available fluorous stationary phases. In many embodiments, the labeling reagents are compatible with protic solvents, making them highly suitable for the analysis of biologically-derived samples. Separation techniques based on the fluorous properties of the labeled species are performed, an approach that is highly orthogonal to classical separation methods currently available (e.g., biological-based interactions, such as that of biotin with (strept)avidin), and thus are less susceptible to the non-specific interactions (and greater costs) associated with biological-based separation techniques. In addition, differently tagged species can often be separated from one another, leading to the potential for the multiplexing of a particular analysis, or the separation of fluorous labeled species having differing numbers of tags.

A fluorous approach to proteomics and metabolomics analysis has several additional advantages over techniques currently available in the art. For example, the fluorous labels employed in the methods of the present invention are typically inert under the low energy (e.g., collision-induced dissociation) conditions used in tandem MS. The mass difference between labeled and unlabeled fragment ions can assist in determination of the site of modification within the protein. Additionally, the mass defect and monoisotopic nature of fluorine can confirm the presence of tagged peptides or other small molecules based solely upon their accurate mass measurement. The fluorous labeled sample members as described herein are typically soluble in mobile phases compatible with electrospray ionization. Optionally, cleavable labels are also provided herein, as are fluorous labeling reagents having stable isotopes incorporated therein. These and other advantages of the present invention are provided in greater detail herein.

The present invention provides various methods for preparing one or more compounds in a biologically-derived sample for analysis using fluorous labeling reagents. In general, the methods of the present invention involve modifying sample components by reacting a sample with a fluorous labeling reagent, thereby incorporating a fluorous label. Optionally, the methods further include separating the modified sample components from unmodified components, and analyzing one or more separated fractions, e.g., by mass spectrometry.

In one aspect, the present invention provides methods for preparing one or more compounds in a biologically-derived sample for analysis. The methods include the steps of providing a fluorous labeling reagent comprising a chemically-reactive functional group coupled to a fluorous moiety comprising five or more fluorine atoms; and coupling the fluorous labeling reagent to one or mote member compounds in the biologically-derived sample via the chemically-reactive functional group to produce fluorous labeled sample components, thereby preparing the biologically-derived sample for further analysis. In some embodiments, the methods further include the step of separating the fluorous labeled sample components from unmodified components using a composition having an affinity for the fluorous labeling reagent.

In another aspect, the present invention provides methods for separating one or more members of a biologically-derived sample, including the steps of reacting the biologically-derived sample with at least one fluorous labeling reagent comprising a chemically-reactive functional group, such as a bioconjugation agent, coupled to a fluorous moiety comprising five or more fluorine atoms, thereby attaching a fluorous label to one or more sample members to form modified sample members; and separating the modified sample members from unmodified sample members using a composition having an affinity for the fluorous label.

In another embodiment, components of a biologically-derived sample having a plurality of amino acid-containing constituents (e.g., such as a proteomics sample) can be prepared for analysis by reacting the plurality of amino acid-containing components with at least one fluorous labeling reagent comprising an amino acid conjugation agent coupled to a fluoroalkyl moiety comprising five or more fluorine atoms, thereby attaching a fluorous labeling reagent to one or more of the amino acid-containing components to form modified amino acid-containing components, and separating the modified amino acid-containing components from unmodified components using a composition having an affinity for the fluorous labeling reagent.

Optionally, as an extension of these methods for preparing and/or separating components of a biologically-derived sample, members of the complex composition (or a fraction thereof) can further be analyzed, e.g., by mass spectrometry.

In a further aspect, the preset invention provides methods for analyzing a complex composition comprising a plurality of biologically-derived components. The analysis methods include the steps of a) providing a fluorous labeling reagent comprising a chemically-reactive functional group coupled to a fluorous moiety comprising five or more fluorine atoms; b) modifying one or more members of the complex composition with the fluorous labeling reagent to form a modified composition comprising fluorous labeled components and unlabeled components; c) fractionating the modified composition using a separating composition having an affinity for the fluorous moiety of the fluorous labeling reagent; and d) performing mass spectrometry on a separated sample fraction and generating mass spectral data, thereby analyzing the complex composition.

Biologically-Derived Samples

The methods of the present invention are performed on one or more biologically-derived samples, including, but not limited to, proteomics and metabolomics samples. These samples are complex compositions having a plurality of components, unlike organic reaction mixtures of chemical syntheses intermediates. As such, the methods of the present invention can be employed with samples having a plurality of sample members, e.g., biologically-derived preparations having at least 25 constituents, or at least 50 constituents, or at least 100 constituents, or at least 1,000 constituents, or even more complex populations of tens of thousands of constituents (for example, at least 10,000 components, 100,000 components, 1 million components, or more).

Biologically-derived samples for use in the present invention can either prokaryotic or eukaryotic in origin. Sources for samples are almost boundless: animal or plant cells; yeast, fungi, bacteria, viruses and/or cells infected with viruses; cell cultures, tissue cultures, or biopsy samples; whole cells or cell lysates; untreated cells, or cells/organisms treated with chemical compositions (e.g., pharmaceuticals) or exposed to one or more environmental factors (heat, light, changes in pH, and the like). Additional exemplary embodiments of biologically-derived samples for use in the present invention include, but are not limited to, cell culture media which has been exposed to a cell/tissue/organism, various bodily fluids, waste products and/or excretions (e.g., blood, serum, urine, saliva, cerebrospinal fluid, interstitial fluid, and the like). Optionally, the samples can be collected from cells (or organisms) that have been treated with one or more members of a compound library. It is not intended that the invention be limited to biologically-derived samples from any particular organism or cell type.

In many embodiments, a cell lysate is used to provide a proteomics or metabolomics sample for use as the biologically-derived sample. Optionally, the sample is treated, e.g., using proteolytic enzymes or chemical cleavage reagents, to generate peptide fragments or to introduce a chemical functionality into a species to be analyzed.

In some embodiments of the present invention, the biologically-derived sample is treated with one or more proteinases prior to coupling the fluorous label to sample members. Alternatively, the proteinase treatment can be performed after fluorous labeling of the sample. Exemplary proteolytic enzymes for use in the present methods include, but are not limited to, trypsin, chymotrypsin, endoprotease ArgC, aspN, gluc, and lysC. Optionally, these proteinases (as well as any additional enzymes not specifically listed) can be used in combination to generate proteolytic fragments of the sample proteins.

Alternatively, members of the biologically-derived sample can be fragmented using a chemical cleavage reagent, such as cyanogen bromide, formic acid, trifluoroacetic acid, or S-ethyl trifluorothioacetate. Chemical cleavage of peptide bonds as well is a process known and described in the art (see, for example, Hunt et al. (1986) Proc. Natl. Acad. Sci. USA 83:6233-6237; and Tsugita et al. (2001) Proteomics 1:1082-1091).

In some embodiments of the present invention, members of the biologically-derived sample are modified to incorporate a specific chemical functionality, to assist in the coupling of the sample member to the fluorous label. For example, the degree and type of post-translational modifications of a sample constituents, particularly phosphorylations and glycosylations, are of interest in the analysis of proteome and metabolome samples. In order to specifically label sample members containing a selected post-translational modification element (for example, a phosphate moiety or a saccharide moiety), the biologically-derived sample may be exposed to reaction conditions which transform the post-translational modification (or a portion thereof) to a specified chemical functionality that can then be reacted with the chemically-reactive functional group of the fluorous labeling reagent.

Optionally, the proteomics sample used in the methods of the present invention is pre-fractionated prior to coupling with the fluorous labeling reagent. Exemplary prefractionated samples include, but are not limited to, gel electrophoresis bands, column. chromatography fractions, and the like.

Fluorous Labeling Reagents

The methods of the present invention employ one or more fluorous labeling reagents These fluorous compositions typically include a chemically-reactive functional group as well as a fluorous moiety having five or more fluorine atoms. Optionally, the chemically-reactive functional group is a portion of a bioconjugation agent, to which the fluorous moiety is to be attached. For example, in embodiments for labeling of peptide constituents of a biologically-derived sample, the fluorous labeling reagent is often a fluorous derivative of an amino acid conjugation agent. While the present invention provides novel fluorous labeling reagents, particularly aqueous-compatible fluorous labeling reagents, the methods of the present invention are not limited to these agents, and as such can be performed with any of a number of known fluorine-containing reagents (such as the fluorine-coupled thiol reagents described in Luo et al. (2001) Science 291:1766-1769).

The present invention provides a straight-forward yet novel approach to simplifying the preparation of proteomic and/or metabolomic samples for further analysis. Because the association properties of fluorous tags are relatively unaffected by the physical characteristics (e.g., molecular weight) of the targeted sample component, varying samples labeled with different fluorous labeling reagents can be processed (e.g., separated, fractionated, and/or analyzed) in a similar manner. As an additional feature, different perfluoroalkyl chains have different retentions that are relatively unaffected by what is attached to them; this property can be employed to perform multiplexed labeling reactions, thereby providing a unique approach to complex composition analysis that cannot be implemented using other methodologies. The fluorous labeling reagents of the present invention provide additional advantages with respect to sample analysis, in that the fluorous labels are typically inert under standard ionization and/or fragmentation conditions used in mass spectrometry, thereby simplifying the data generated during analysis of the labeled species (e.g., little loss of signal due to label fragmentation). Furthermore, the mobile phases used in fluorous chromatography (typically MeOH/water) are compatible with analysis techniques such as ESI.

Fluorous Moieties

Typically, the fluorous labeling reagents of the present invention contains at least one fluorous moiety having five or more fluorine atoms. Exemplary fluorous moieties for use in the present invention are depicted in Table 1. Optionally, the compositions of the present invention have at least six, seven, eight, nine, ten, eleven, twelve, thirteen, fifteen, seventeen, twenty, or more fluorine atoms. In some composition embodiments, the fluorine atoms are coupled to contiguous carbon atoms (e.g., perfluoroalkyl chains). Alternatively, the fluorous moiety can be provided as two or more “clusters” of carbon-coupled fluorine atoms separated by non-fluorous chemical regions. For example, the fluorous moieties of the present invention include, but are not limited to, varying combinations of 'CF₂—, —CF₂CH₂—, and —CFH— elements, either linear or branched, and optionally interspersed with non-fluorous —CH₂— elements. Optionally, other halogens can also be incorporated (in addition to the fluorine atoms) into the compositions of the present invention.

TABLE 1
Exemplary fluorous moieties

| | | |
|---|---|---|
| —CF₂CF₂CF₃ | —(CF₂)₃CH₃ | —CF(CF₃)₂ |
| —CF₂CF₂CF₂CF₃ | —(CF₂)₄CH₃ | —C(CF₃)₃ |
| —CF₂CF₂CF₂CF₂CF₃ | —(CF₂)₅CH₃ | —CF₂CF(CF₃)₂ |
| —CF₂CF₂CF₂CF₂CF₂CF₃ | —(CF₂)ₙCH₃ | —CF₂C(CF₃)₃ |
| —(CF₂)ₙCF₃ | (CH₂)ₙ(CF₂)ₙ(CH₂)ₙ— | [CF₂O]ₙ |

Where n is an integer between 2 and 20.

In some embodiments, the fluorous labeling reagents are fluorous analogs of standard bioconjugation agents (i.e., in which a number of carbon-bound hydrogens typically present in the reagent have been replaced with at least five fluorine atoms). In other embodiments of the present invention, the fluorous moiety portion of the labeling reagent is an additional component coupled to a bioconjugation agent (or portion thereof that bears the chemically-reactive functional group). Optionally, the fluorous moiety is coupled to the chemically-reactive functional group via a linker region, to form the fluorous labeling reagent. Typically, the linker region is an alkyl chain at least two, and optionally between two and twenty, carbons in length. While a linear alkyl chain is provided in the exemplary embodiment, branched alkyl chains and/or aromatic linker elements can also optionally be used.

Furthermore, in some embodiments, the fluorous portion of the labeling reagent and/or the optional linker region includes an isotopic label. Exemplary isotopes for use as isotopic labels in the compositions of the present invention include, but are not limited to, one or more deuterium (²H), ¹³C, ¹⁵N, and/or ¹⁸O atoms.

In a further embodiment, the linker region(s) employed in the compositions of the present invention can include a releasable element, such that the modified sample member or component can be separated from the fluorous moiety at a selected point during processing (e.g., during a separation or fractionation step). Biochemical structures having an enzymatic cleavage site can be used as linker elements in the compositions and methods of the present invention. For example, an oligopeptide representing a protease recognition site, or an oligonucleotide having a restriction site, can be used as linkers between the conjugation agent and the fluorous moiety. Alternatively, releasable elements that are sensitive to chemical cleavage, photolysis, or thermal degradation can be used to release the fluorous moiety from the remainder of the fluorous labeling reagent.

In some embodiments of the present invention, multiple fluorous moieties (with or without accompanying linker elements) are incorporated into the fluorous labeling reagents. For example, a fluorous labeling reagent of the present invention could include a first fluorous moiety coupled at a first position on the bioconjugation agent (e.g., a first position relative to the chemically-reactive functional group), and a second fluorous moiety coupled at a second position on the bioconjugation agent.

In preferred embodiments, the fluorous labeling reagents of the present invention are compatible with aqueous reaction conditions (e.g., the reactive nature of the label is such that the solvent does not out-compete the target species for reaction with the label, so the labeling reagent can be used in the presence of equimolar (or greater) concentrations of H₂O).

Optionally, the hydrophilic nature of the fluorous labeling reagent is further adjusted, as compared to a corresponding nonfluorous bioconjugation agent. This can be achieved, for example, by the addition of hydrophilic groups to the fluorous moiety, or to an optional linker coupling the fluorous moiety to the bioconjugation agent. In some instances, modifications can be made to the bioconjugation agent itself, to increase the aqueous compatibility of the fluorous labeling reagent. For example, the presence of bases can remove active hydrogen species and increase the aqueous compatibility of certain reagents, such as fluorous thiols used in the β-elimination reactions. Additionally, N-hydroxysulfosuccinimidyl esters of various mono- and di-carboxylic acid-containing fluorous reagents can be used to increase the aqueous compatibility of these amine-reactive reagents. In general, peptides labeled with fluorous reagents remain completely aqueous compatible.

Chemically-Reactive Functional Groups

In addition to the one or more fluorous moieties described above, the fluorous labeling reagents of the present invention include at least one chemically-reactive functional group (which in some embodiments, represents only a portion of the “bioconjugation agent”). The chemically-reactive functional group is used to couple the fluorous label to the targeted species in the biologically-derived sample.

The targeted species within the biologically-derived sample typically contain within their structure a common functionality (the “targeted chemical functional group” or “reactive group”). For example, for amino acid-containing species, the targeted chemical functional group can be either a portion of the amino acid itself (e.g., an amino acid side chain), or a functionality coupled to or otherwise associated with the amino acid, such as a post-translational modification. In some embodiments, the functional group to be targeted is not normally part of the native molecule, but is generated by derivatization of the sample members for the purpose of tagging with the fluorous labeling reagent.

Targeted chemical functional groups to be reacted with the fluorous labeling reagents of the present invention (e.g., common functional groups found within sample members of the biologically-derived sample) include, but are not limited to, a sulfhydryl group, a thioether group, an amino group, a carboxyl group, a hydroxyl group, a ketone or aldehyde, an imidazole group, a guanidino group, or an indole moiety. In some embodiments of the present invention, the chemically-reactive functional group portion of the fluorous labeling reagent is selected to react with an unmodified side chain of an amino acid. In other embodiments, the chemically-reactive functional group targets the fluorous label to a post-translational modification element (for example, a phosphate moiety or a saccharide moiety), or a product resulting from the removal or other chemical transformation of the post-translational modification.

Exemplary embodiments of these and other fluorous labeling reagents are provided in Table 2 and in the Examples. The embodiments illustrated herein are intended to serve only as examples; it is not intended that the invention be limited to any particular fluorous moieties illustrated herein. After reading a description of the invention, a variety of embodiments will be apparent to one of skill in the art, all of which are encompassed by the scope of the claimed invention,

TABLE 2
Exemplary fluorous labeling reagents

| Compound | Structure |
|---|---|
| 1a | CF₃(CF₂)₇CH₂CH₂SH |
| 1b | CF₃(CF₂)₅CH₂CH₂SH |
| 1c | CF₂H(CF₂)₅CH₂CH₂SH |
| 1d | (CF₃CF₂)₂CF(CF₂)₂CH₂CH₂SH |
| 1e | (CF₃CF₂)₂CH(CF₂)₂CH₂CH₂SH |
| 1f | CF₃(CF₂)₃CH₂CH₂SH |
| 2a | 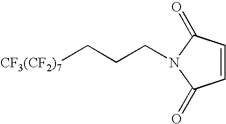 |
| 2b | 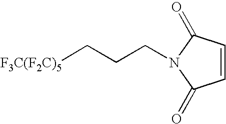 |
| 3 | 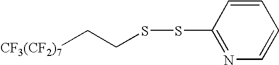 |
| 4a | 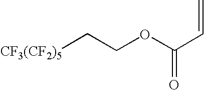 |
| 4b | 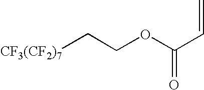 |
| 5a | 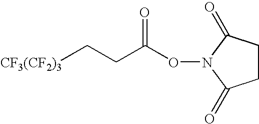 |
| 5b | 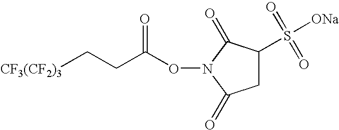 |
| 5c | 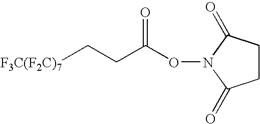 |
| 5d | 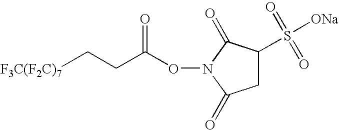 |
| 5e | 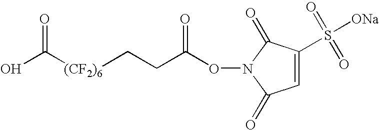 |
| 6a | 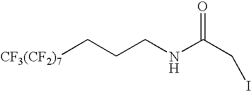 |
| 6b | 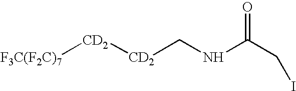 |
| 6c | 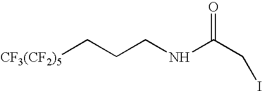 |
| 6d | 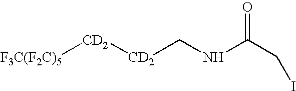 |
| 7 | 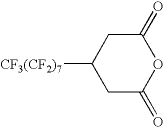 |
| 8 | 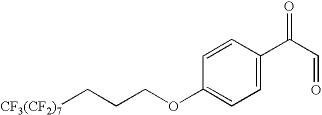 |
| 9 | 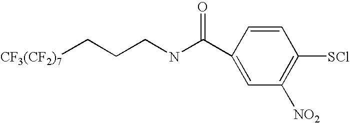 |
| 10 | 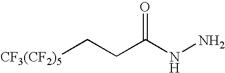 |
| 11 | 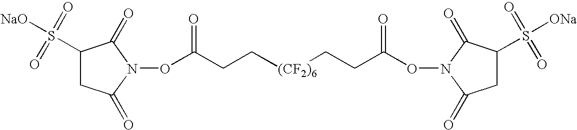 |
| 12 | 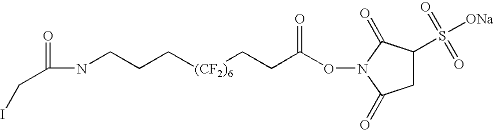 |
| 13 | 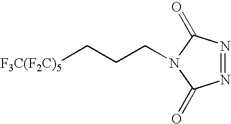 |
| 14 | 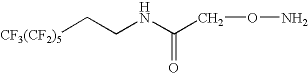 |
| 15a | 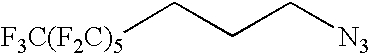 |
| 15b | 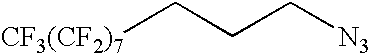 |
| 16a | 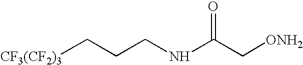 |
| 16b | 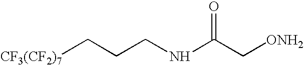 |
| 16c | 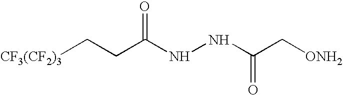 |
| 16d | 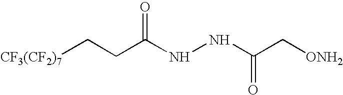 |
| 17 | CF₃(CF₂)₅CH₂CH₂CH₂NH₂ |
| 18 | 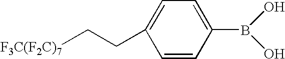 |
| 19a | 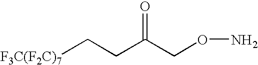 |
| 19b | CF₃(CF₂)₅CH₂CH₂(C═O)CH₂ONH₂ |
| 19c | CF₃(CF₂)₃CH₂CH₂(C═O)CH₂ONH₂ |
| 20 | 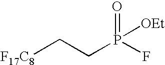 |
| 21 | 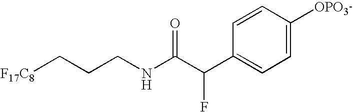 |
| 22 | 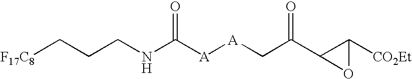 |
| | wherein A = amino acid |
| 23 | 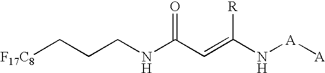 |
| | wherein A = amino acid |
| 24 | H₂NCH₂CH₂CH₂(CF₂)₆CH₂CH₂SH |
| 25 | 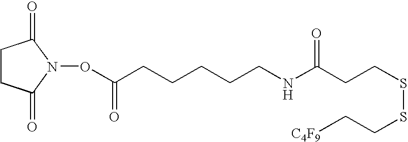 |

Fluorous Labeling of Sulfhydryl Groups

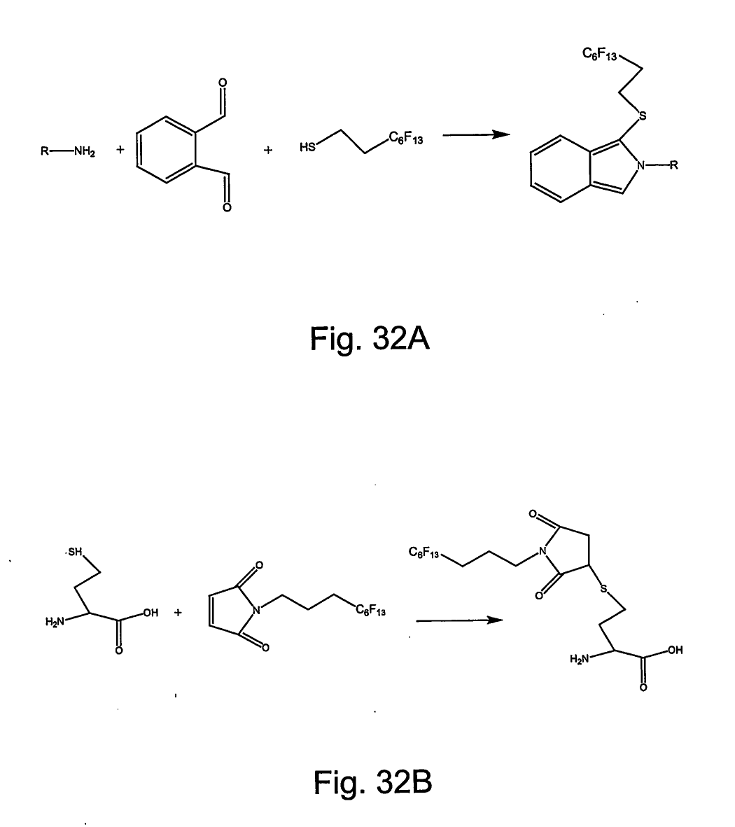

Sulfhydryl groups are among the most highly reactive functionalities present in biomolecules. Alkylation or disulfide exchange reactions are typically used for bioconjugation of sulfhydryl-containing molecules (e.g., cysteine side chains). For example, maleimides and acrylate Michael acceptors can be used to irreversibly alkylate sulfhydryl groups by forming a stable thioether bond (see, for example, the fluorous labeling of homocysteine depicted in FIG. 32B). As such, these chemically-reactive functional groups are suitable for use in the methods and compositions of the present invention. Exemplary maleimide-type fluorous labeling reagent for use as in the present invention include, but are not limited to, N-(3-(perfluorooctyl))propylmaleimide 2a and N-(3-(perfluorohexyl))propylmaleimide 2b. Other Michael acceptor-type fluorous labeling reagents for labeling sulfhydral moieties include, but are not limited to, 1H, 1H, 2H, 2H-perfluorooctyl acrylate 4a and 1H, 1H, 2H, 2H-perfluorodecyl acrylate 4b.

In another embodiment, activated halogen derivatives, such as haloacetals, benzyl halides, and alkyl halides (e.g., halogen β-ketones) are employed as chemically-reactive functional groups in the compositions of the present invention. An exemplary halogen β-ketone-type fluorous labeling reagent for use in the present invention is N-iodoacetyl-3-(perfluorooctyl)propylamine 6a.

In yet another embodiment, disulfide exchange reagents are used as the chemically-reactive functional group in the compositions of the present invention. The disulfide exchange/interchange reaction involves a bioconjugate agent having a disulfide bond incorporated therein; a sulfhydryl moiety present in a sample member is then able to attack the disulfide moiety of the fluorous labeling reagent, breaking the bond and forming a new, reversible (cleavable) coupling between the labeling reagent and sample member. An exemplary disulfide exchange reagent-type fluorous labeling reagent of the present invention is 2-pyridyl-2′-1H,1H,2H,2H-perfluorodecane disulfide 3.

Linear and branched thiol species can also be employed as fluorous labeling reagents in the present invention. In addition to their usefulness in exploring fluorous biophysical parameters (such as the relationships between total fluorine content, 3-dimensional orientation of the fluorine atoms and retentive properties), thiol-type fluorous compositions such as those depicted in Table 1 (e.g., compounds 1b and 1c, and compounds 1d and 1 e), could be used as “mass coded affinity tags.” Assuming that the substitution of a hydrogen for a fluorine atom within the fluorous moiety does not cause a significant difference in relative retention times between the differently labeled analogs, use of differentially fluorinated labeling reagents would enable a relative quantitation scheme not requiring the incorporation of any stable isotopes, where pairs of MS peaks would be shifted by 18Da (i.e., the difference in mass between F and H).

Fluorous Labeling of Amino and/or Guanidino Groups

Nitrogen-containing moieties such as amino and guanidino groups are also reactive functionalities that can be targeted for modification within a biologically-derived (e.g., proteomic or metabolomic) sample.

For example, acylation reactions under properly controlled conditions have been shown to be effective for bioconjugation of amine-containing molecules (e.g., lysine side chains, as well N-terminal amino groups). N-hydroxysuccinimide (NHS) derivatives, and more particularly hydrophilic sulfo-NHS derivatives, can be used to acylate the amino groups in a peptide or protein sequence. Exemplary fluorous derivatives of these acylation reagents include, but are not limited to succinimidyl-2H,2H,3H,3H-perfluoroheptanoate 5a and sulfosuccinimidyl-2H,2H,3H,3H-perfluoroheptanoate 5b, as well as the corresponding nonanoate and undecanoate derivatives.

Alternatively, fluorous anhydride derivatives such as 3-(perfluorooctyl)glutaric anhydride 7 can also be employed as amino-targeting fluorous labeling reagents in the present invention.

For the purpose of labeling guanidino-type nitrogen moieties in a sample, fluorous 1,2-dicarbonyl reagents, such as 4-[3-(perfluorooctyl)propyl-1-oxy]phenyl glyoxyl 8, can be employed. These reactants undergo a condensation reaction with target guanidino moieties (such as the guanidino side chain of arginine).

Fluorous Labeling of Keto and/or Aldehyde Moieties

Keto and/or aldehyde moieties are yet another reactive functionality naturally present in some biomolecules of interest (e.g., ketone groups on steroidal derivatives, various pharmacological intermediates or degradation products), or can be introduced into select sample members of interest by a number of processes. These chemical functionalities can be targeted for fluorous labeling, for example, using fluorous derivatives of hydrazine (to form fluorous labeled hydrazides) or amino-oxy compounds (to form fluorous labeled oximes).

For example, one approach to selectively labeling carbohydrate-modified peptides in a biologically-derived sample is to generate a reactive aldehyde moiety by performing a chemical oxidation (e.g., using sodium periodate), or using a specific sugar oxidase. The aldehyde is then labeled with a hydrazine-type fluorous labeling reagent such as 10 (as opposed to using a biotin-hydrazide complex, a more expensive approach which is more susceptible to non-specific interactions). Another approach is to react the aldehyde with an aminooxy-type fluorous labeling reagent, to form the oxime. The fluorous labeled species can then be isolated and further analyzed.

In a further embodiment of the methods of the present invention, carbodiimide derivatives in combination with a fluorous amine (i.e. 3-(perfluorooctyl)propylamine 17) or fluorous alcohol (i.e., 3-(perfluoroheptyl)propan-1-ol) are used for fluorous labeling of carboxyl moieties in the biologically-derived sample. In these methods, the carboxylic acid moieties of sample members (e.g., amino acid-containing sample members) are converted to the corresponding fluorous amide or ester.

Bioconjugation of Methionine Side Chains

Amino acid sequences having methionine residues can also be fluorous labeled. The methionine side chain reacts with halogen β-ketones in a manner similar to that described for cysteine residues, except the methionine labeling reaction is typically performed at acidic (2-3) pH. Optionally, the resulting bond can be cleaved to give back the methionine-containing peptide on reaction with a thiol such as B-mercaptoethanol. An exemplary fluorous labeling reagent for use with methionine residues is N-iodoacetyl-3-(perfluorooctyl)propylamine 6a.

Bioconjugation of Indole Groups

The indole moiety of tryptophan residues can be reacted with various sulfenyl halides to introduce a sulfenyl group at the 2-position on the indole ring. Tryptophan-targeting fluorous labeling reagents of the present invention include, but are not limited to, fluorous sulfenyl halides, such as 2-nitro-4-(N-(3-(perfluorooctyl)propyl)carboxamide)benzenesulfonyl chloride 9.

Other Chemical Ligation Approaches

Additional fluorous labeling reagents can be prepared based upon a variety of selective chemical ligation reactions, thus targeting a number of other (natural or introduced) chemical functionalities within a sample population, including, but not limited to, cis-dienes, alkynes, and vicinal diols. For example, two highly orthogonal chemical ligation strategies for which fluorous reagents can be prepared are the “Staudinger Ligation” (for targeting of phospane-bearing species; for a review, see Köhn and Breibauer (2004) Ang. Chem. Int. Ed. 43:3106-3116) or Huisgen 1,3-dipolar cycloaddition-type ligation reactions (“click” chemistry for targeting alkyne-bearing substrates; see Rostovtsev et al. (2002) Angew Chem Int Ed 41:2596-2599 and references cited therein). These methodologies are becoming increasing popular in a variety of proteomics applications ranging from the isolation of specific species from complex mixtures to the ordered arraying of target species. Exemplary fluorous azides for use in targeting of alkyne-containing biologically-derived sample components are provided in Table 2.

In a further embodiment of the present invention, fluorous labeling reagents are provided that include a “suicide inhibitor” as the chemically-reactive functional group. These fluorous reagents can be used to selectively target active enzymatic species in a biologically-derived sample (e.g., activity-based proteomics studies).

The chemically-reactive functional group employed in the suicide-type fluorous labeling reagents typically fall into one of two general categories: small peptide structures and simple but highly orthogonal small molecules prepared, e.g., by rational drug design. For example, serine hydrolases in a biologically-derived sample can be targeted using fluorous reagents such as 20 and various sulfonate ester analogs. Fluorous labeling reagent 21 can be used to specifically target tyrosine hydrolases, or fluorous labeling reagents 22 or 23 for targeting of cysteine proteases (see, for example, Greenbaum et al. (2000) Chemistry and Biology 569; Winssinger et al. (2001) Ang. Chem. Int. Ed. 40:3152). Additional exemplary reagents for use in the design and preparation of additional “suicide-type” fluorous labeling reagents are provided, for example, by Liu et al. (1999) “Activity-based protein profiling: the serine hydrolases” Proc. Natl. Acad. Sci USA 96:14694-14699.

Multi-Functional Labeling Reagents

Optionally, the fluorous labeling reagents of the present invention can further include an additional chemically-reactive functional group for derivatizing an additional amino acid-associated functional group. In these embodiments, the fluorous moiety acts as a linker between the two chemically-reactive functional groups. Either similar or dissimilar functional groups can be targeted by the first and second functionalities. Thus, for composition embodiments having multiple chemically-reactive functional groups, the bioconjugation elements need not be of the same structure or have an affinity for the same type of amino acid residue (i.e., the two-pronged fluorous labeling reagent can be used to couple disparate chemical entities).

An exemplary homofunctional crosslinking fluorous labeling reagent having similar targeting specificities (i.e., both chemically-reactive functional groups in the fluorous labeling reagent are capable of reacting with the same functional group) is bis(sulfosuccinimidyl)-2H,2H,3H,3H,10H,10H,11H,11H-perfluorododecanedionate 11, an amine-targeting composition.

An exemplary heterofunctional crosslinking fluorous labeling reagent that reacts with different functional groups in a sample is sulfosuccinimidyl-12-[(iodoacetyl)amino]2H,2H,3H,3H,10H,10H,11H,11H, 12H,12H-perfluorododecanoate 12 (which compound is both amine and thiol reactive).

Modifying and Separating Proteomics Sample Components

After providing the proteomics sample and fluorous labeling reagent(s), the next step in the analytical methods of the present invention involves modifying one or more components of the proteomics sample (e.g. members of the plurality of amino acid-containing components). By incorporating one or more fluorous labels into the targeted members of the proteomics sample and forming modified proteomics sample components, the self-association properties of fluorous-containing compounds can be put to use in the separating steps of the methods as provided herein.

As noted above, a fluorous version of any of a number of common amino acid conjugation agents (e.g. labeling reagents) can be synthesized and used for isolation of the labeled peptides from the remaining bulk of unlabeled peptides. The fluorous-containing amino acid conjugation agent interacts with the peptide component such that the portion of the agent having the fluorous label becomes associated with the modified peptide. In some embodiments of the methods of the present invention, the fluorous labeling reagents is composed of a plurality of fluorous labeling reagents. For example, the labeling reagent can have a first amino acid conjugation agent coupled to a first fluorous moiety, and as a separate chemical entity, a second amino acid conjugation agent coupled to a second fluorous moiety. In such embodiments involving a plurality of fluorous labeling agents, preferably the first and second fluorous moieties differ in their affinity for the separating composition.

In an alternative embodiment, the fluorous labeling reagent employed in the methods has two amino acid conjugation agents (e.g., a first amino acid conjugation agent and a second amino acid conjugation agent) coupled via a fluoroalkane linker. An exemplary fluoroalkane linker is represented by the formula
—CH₂CH₂(CF₂)ₙCH₂CH₂—
wherein n is an integer between 3 and 20.

Since fluorous moieties tend to associate primarily with “like” or simnilar compositions, this property is utilized in the separating step of the methods of the present invention. Segregation between fluorous-containing and non-fluorous containing compositions occurs fairly independent of the nature (e.g., size) of the species attached to the labels. As an added feature, chemical species having fluorous labels of different fluorous compositions can be separated from each other with specific retention properties that persist regardless of the nature of the bound species.

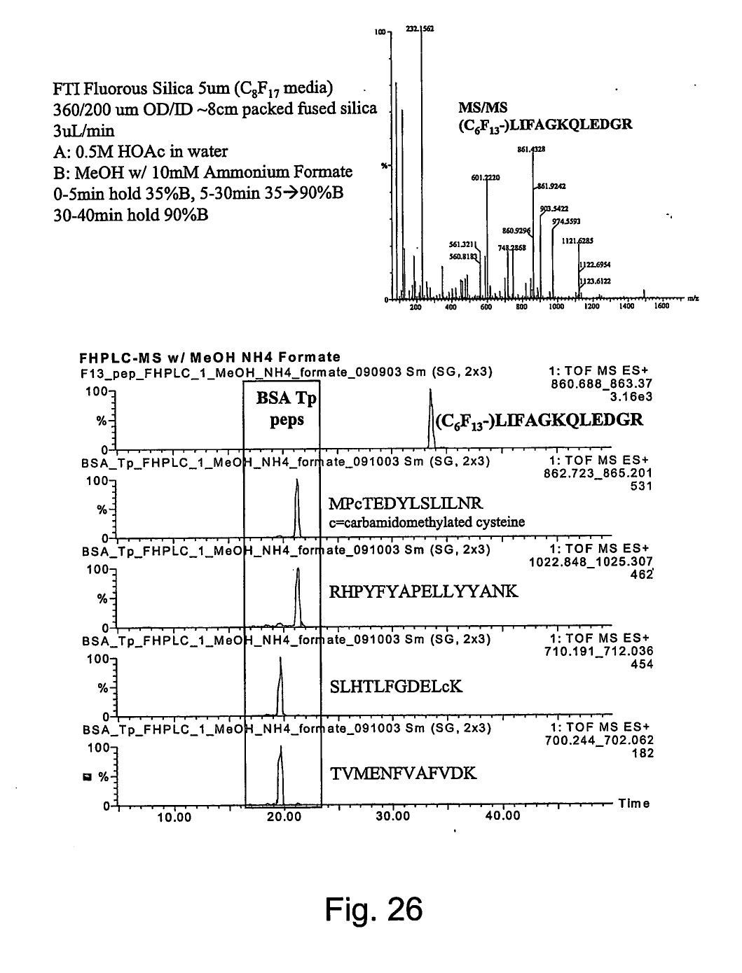

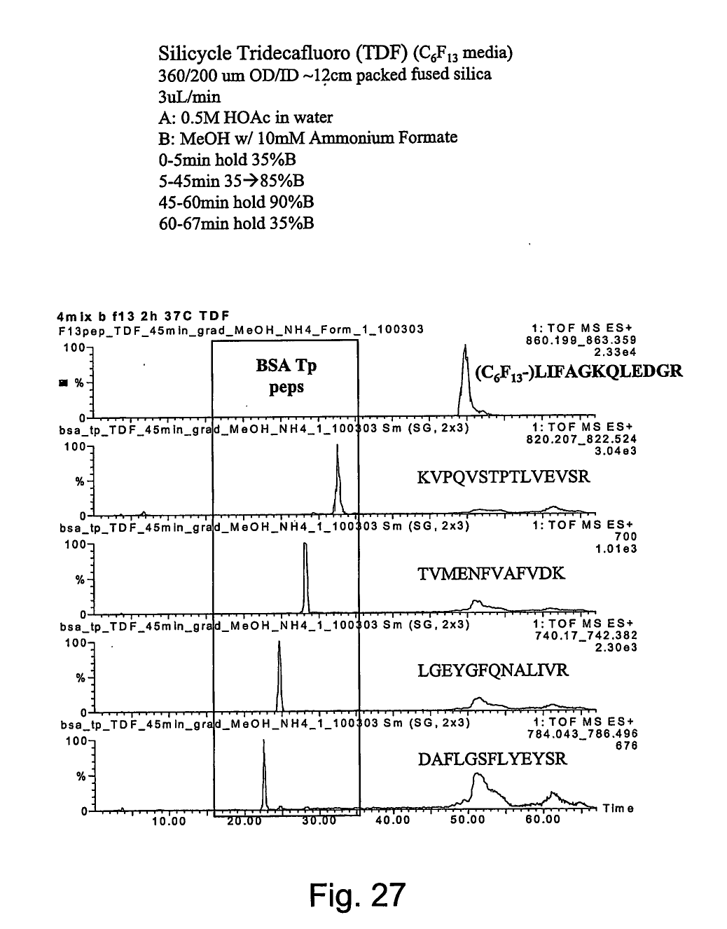

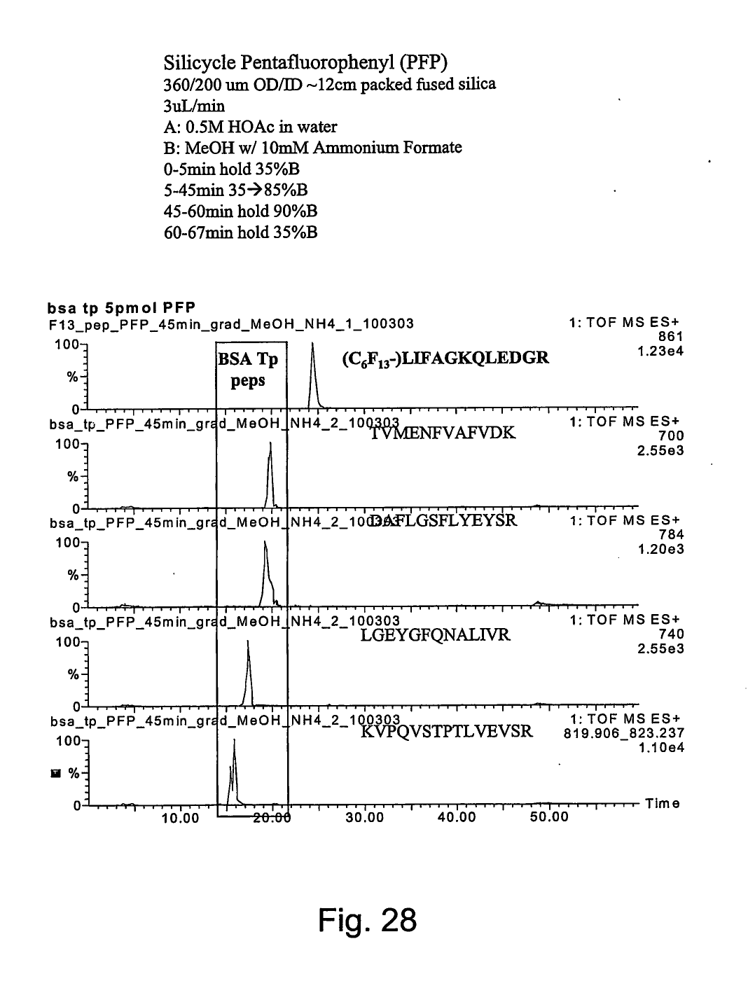

A further step in the methods of the present invention involves separating the modified (e.g., fluorous labeled) proteomic sample components from unmodified components using a composition having an affinity for the fluorous label. Fluorous separations are typically highly selective with minimal backgrounds and are relatively simple to implement. It should be noted that the ability to distinguish fluorous tagged species from unlabeled species can also be affected by choice of the stationary phase (see, for example, FIGS. 26-28).

Any of a number of fluorophilic compositions can be used to separate the fluorous labeled and non-labeled (unmodified) proteomics sample components. For example, a number of fluorous stationary phases or fluorous affinity matrices can be prepared by coupling fluorous moieties to silica gel or a polymeric substrate (e.g., polystyrene), or by polymerizing fluorous monomers. In a preferred embodiment, the composition having an affinity for the fluorous label is fluorous silica gel (e.g., FluoroFlash® Silica Gel from Fluorous Technologies Inc., Pittsburgh, Pa.). Alternatively, fluorous solvents such as FC-72® from 3M (Maplewood, Minn.) can be used in liquid:liquid or liquid:solid extraction techniques.

Optionally, separating the modified proteomic sample components from unmodified components can be achieved by performing fluorous-based separation technique (e.g., batch-style solid phase extraction using, e.g., a fluorous-functionalized stationary phase, or fluorous column chromatography) using a fluorous affinity matrix, and collecting a column effluent to be further analyzed. The column effluent can be either the unbound (e.g., non-fluorous) portion of the proteomics sample, or a fluorous-containing fraction.

In a further aspect, the present invention also provides methods for fractionating fluorous and non-fluorous components of a fluorous labeled sample directly on a surface of a substrate (for example, a MALDI or DIOS plate, e.g., for sample clean-up directly on the sample plate). A composition having an affinity for a fluorous label is coupled to a first portion of a surface of the substrate, to form a fluorous 2-dimensional surface on the surface. The fluorous affinity composition can cover either the entire surface of the substrate, or select portions of the surface (e.g., an array of positions spread across the surface of the substrate). As a further example, a fluorous-modified porous silicon surface can be prepared for use with the DIOS (desorption ionization on silicon) methodology described by Wei et al. in “Desorption/Ionization Mass Spectrometry on Porous Silicon” (1999) Nature 399:243-246.

The (unfractionated) fluorous labeled sample is then loaded directly onto the substrate surface, after which the fluorous components and nonfluorous components can be separated based upon their affinity for the fluorous affinity composition. For example, separating the fluorous-labeled components from the nonfluorous components could involve the steps of associating the fluorous components of the sample with the composition having an affinity for the fluorous label and thereby localizing the fluorous components to the surface, followed by removal of any nonfluorous components e.g., by washing.

Mass Spectroscopy

The methods of the present invention further include the step of performing mass spectrometry on a separated component, thereby analyzing the proteomics sample. While a number of mass spectrometry techniques can be used in the present invention, tandem MS is particularly useful. Preferably, the fluorous labeling reagents employed in the methods of the present invention are chemically stable compositions, such that the fluorous moieties are inert under mass spectroscopy conditions for low energy collisions (e.g., collisionally-activated dissociation (CAD) conditions).

In many embodiments of the present invention, the mobile phases used in the fluorous-based separating step are mixtures of water- and methanol. This solvent system is compatible with ESI techniques, giving rise to the possibility of direct elution of species from the fluorous column into the mass spectrometer.

In one embodiment, analysis is performed by collecting mass spectral data for both a separated fraction of the proteomics sample as well as an untreated portion of the sample. The MS data are then compared. The separated fraction can be either a non-retained (e.g., unlabeled) portion of the proteomics sample, or a retained (fluorous labeled) fraction. For embodiments in which the separated component is an unmodified proteomics sample component (e.g., a fluorous column flow-through fraction), comparing the MS data can include determining which MS peaks are present in the original untreated proteomics sample but not in the unmodified proteomics sample component (i.e., which peaks have been retained by the fluorous affinity matrix). For embodiments in which the separated fraction is a fluorous-containing fraction, the methods optionally further include the step of separating singly-labeled member components from multiply-labeled member components.

Some embodiments of the methods of the present invention were performed using a Bruker Biflex III MALDI TOF instrument or a Micromass ESI Q-TOF-2 Instrument. Optionally, methods and systems for identification of proteins using high mass accuracy mass spectrometry, such as those described in PCT publication WO 03/054772 to Brock et al. (“Methods and Devices for Proteomics Data Complexity Reduction”) can be used in the analysis step of the methods provided herein. Experiments involving high mass accuracy (such as for the analysis of shifted isotopic distribution of the tagged species) were performed using a modified 7.0 T Bruker Apex II FT-ICR instrument, equipped with a home-built MALDI source, a new open-cylindrical cell, and a quadrupole mass spectrometer (ABB Extrel). High mass accuracy measurements provide greater confidence in protein identification assignments and enable proteins to be identified with less sequence coverage (e.g., fewer peptides) and fewer additional tandem MS experiments.

An accurate mass measurement of the observed peptides can be also utilized to advantage in the analysis process. Fluorine has a mass of 18.9984 amu. If measured accurately, the mass of a fluorine :moiety-containing derivative will be less than its calculated nominal mass. In contrast, the accurate mass of a non-fluorous labeled compound having the same nominal mass will be slightly higher than the calculated nominal mass. Thus, accurate mass measurements, when compared to calculated nominal mass values for a series of theoretical compositions, can be used to determine whether a given MS peak represents a fluorous labeled species. Methods for accurate mass determination are provided, for example, in PCT publication WO 03/054,772 by Brock et al., titled “Methods and Devices for Proteomics Data Complexity Reduction.”

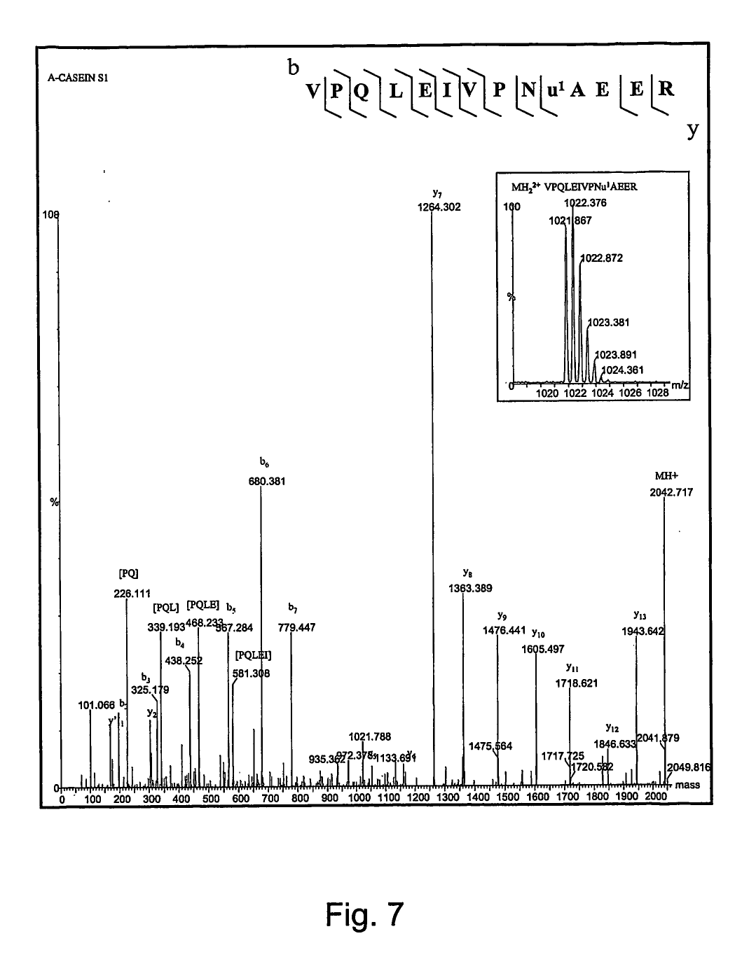

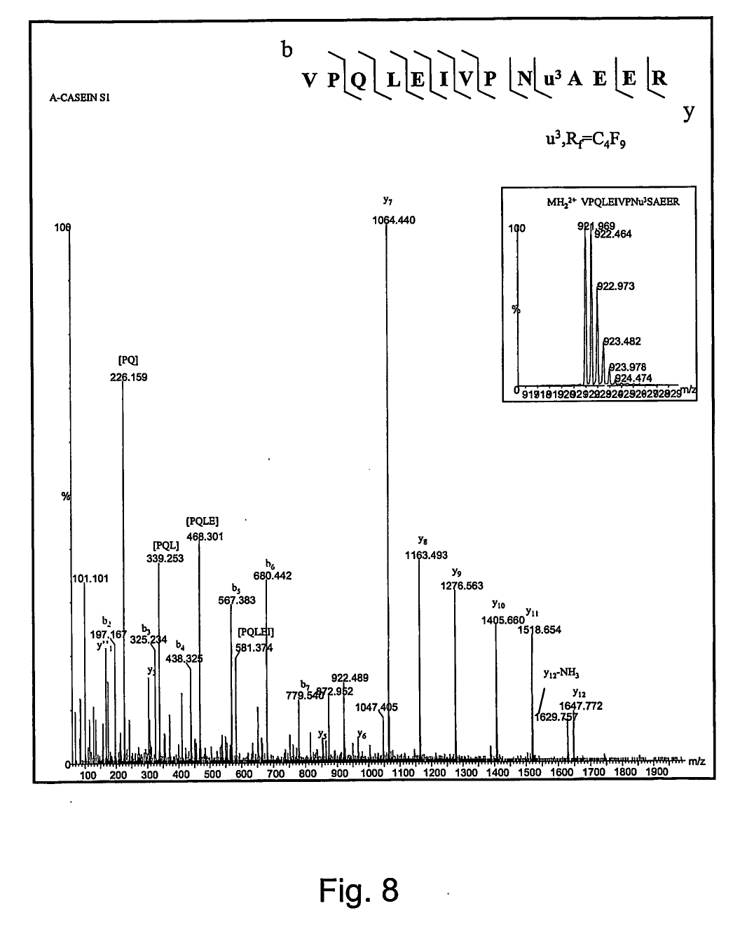

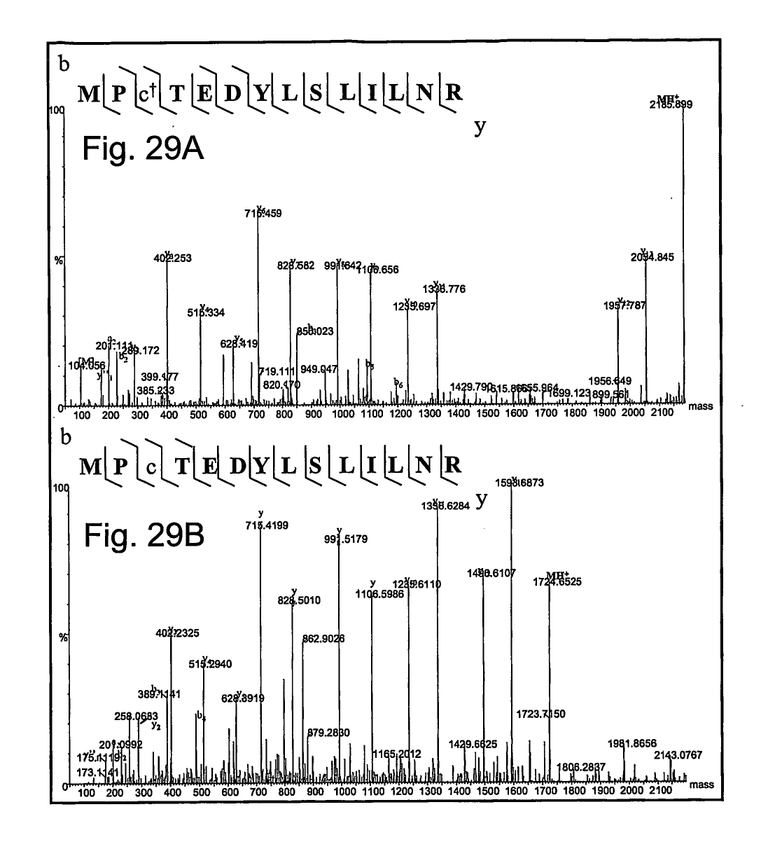

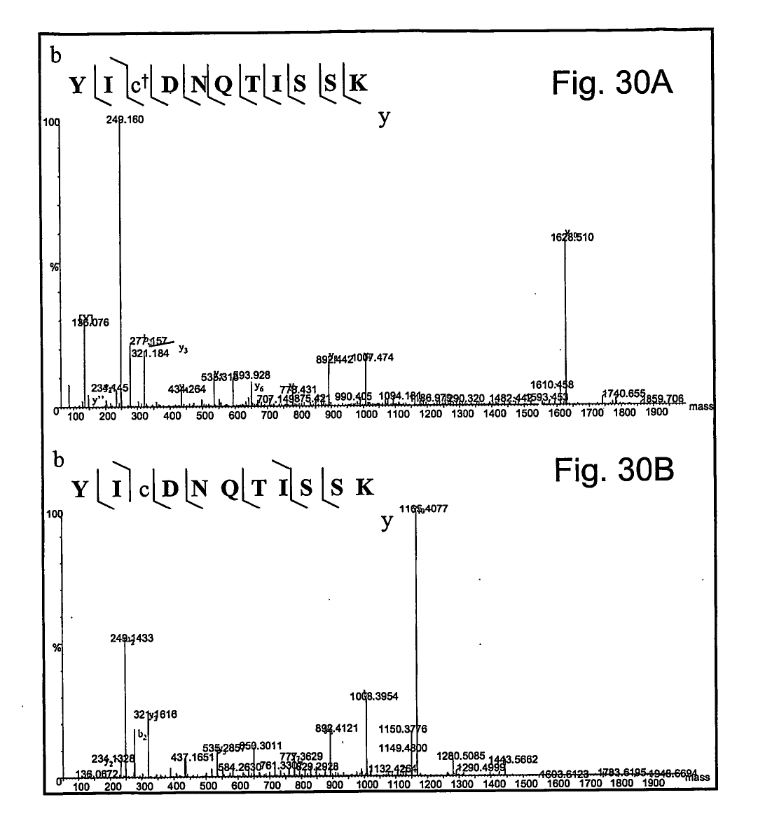

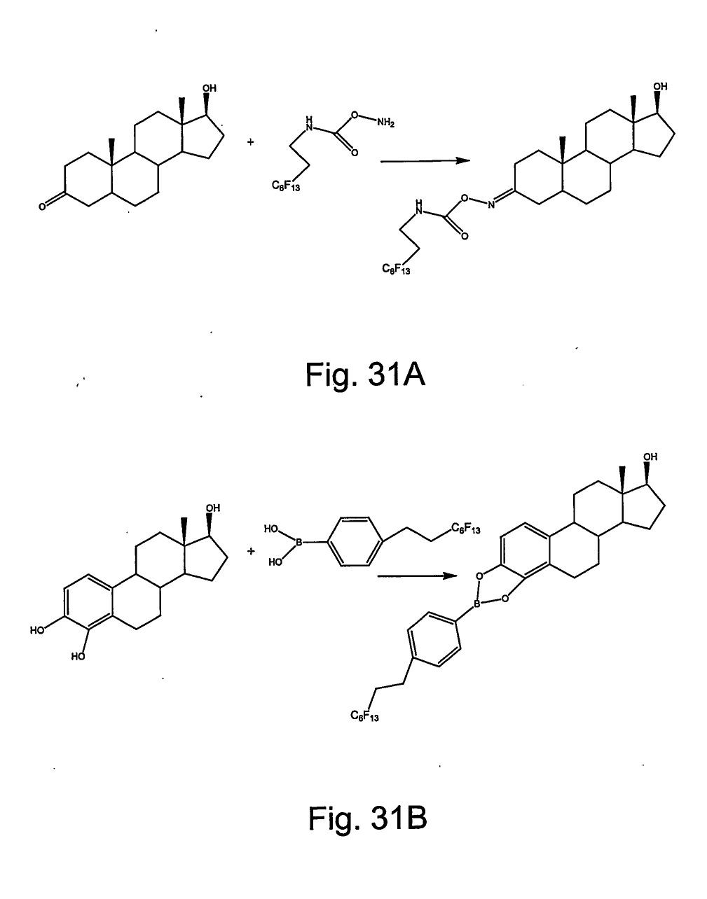

FIGS. 29 and 30 provide two comparative examples of inertness of a fluorous tag in tandem MS, comparing the tandem MS pattern of a native cysteine-containing peptide and its acrylate-labeled counterpart. In both cases, no distinctive peaks due to the decomposition of the tag are observed (in contrast to an alternative labeling reagent, the ICAT reagent). Similarly, FIGS. 7 and 8 demonstrate the inertness of fluorous tags under tandem MS conditions (two identical species with different sized tags issuing spectra that are identical except due to mass shifts due to the different labels).

Analysis of Post Translational Modifications

The methods of the present invention can be used, for example, to examine changes in post-translational modification(s) of proteomics or metabolomics sample components. Post translational modifications (PTMs) include, but are not limited to, glycosylation, phosphorylation, sulfation, fatty acid attachment, and the like.

In some embodiments, the methods of the present invention are used to analyze members of a plurality of amino acid-containing components having at least one phosphorylated component. The phosphorylated component can include one or more phosphorylated serine residues, one or more phosphorylated threonine residues, or a combination thereof. Modifying phosphorylated members of the plurality of amino acid-containing components can be performed, for example, by a) adding a base to the plurality of amino acid-containing components to form a reaction mixture; b) performing a β-elimination reaction on the phosphorylated component; c) adding the fluorous labeling reagent to the reaction mixture; and d) performing a Michael addition reaction on a product of the β-elimination reaction. This procedure results in the coupling of a fluorous label at the (previous) site of phosphorylation and generating a fluorous labeled proteomic sample component. An exemplary fluorous labeling reagent for the described application is 1H, 1H,2H,2H-perfluorodecane-1-thiol.

In other embodiments of the present invention, the methods are used to analyze one or more glycosylated proteomics components. For example, sugar hydroxyl functionalities can be acylated or alkylated using fluorous-containing reagents. In one embodiment, the step of modifying the glycosylated members of the proteomics sample include the steps of a) oxidizing one or more sugars on the glycosylated component to generate one or more aldehyde moieties in a reaction mixture; b) adding the fluorous labeling reagent to the reaction mixture, wherein the amino acid conjugation agent comprises a hydrazide-containing compound; and c) coupling the aldehyde moieties with the hydrazide through a hydrazone bond. Optionally, the hydrazone bonds are reduced, thereby generating a fluorous labeled amino acid-containing component.

Oxidizing the one or more sugars in the glycosylated proteomics component can be performed by a number of techniques known in the art. For example, a periodate oxidation reaction can be used to introduce an aldehyde functionality in sugars having two adjacent hydroxyl groups. An exemplary fluorous labeling reagent for the described application is 2H,2H,3H,3H-perfluononanoic acid hydrazide.

Data Set Comparisons

In another aspect, the present invention provides methods for analyzing a proteomics sample including the steps of: a) providing a proteomics sample having a plurality of amino acid-containing components (proteins, peptides, and the like); b) providing a fluorous labeling reagent having an amino acid conjugation agent coupled to a fluorous moiety having five or more fluorine atoms; c) reacting the proteomics sample with the fluorous labeling reagent to form a treated proteomics sample, thereby incorporating a fluorous label into one or more member components of the proteomics sample and forming fluorous modified (i.e. labeled) components; d) analyzing a first portion of the treated proteomics sample by mass spectrometry and generating a first set of mass spectral data; e) analyzing a second portion of the treated proteomics sample by mass spectrometry and generating a second set of mass spectral data, wherein the fluorous modified components of the second portion have been removed by fluorous-based separation techniques using a fluorous affinity matrix prior to analyzing; and f) comparing the first and second sets of mass spectral data and determining one or more mass spectral peaks which are present in the first portion and absent in the second portion, thereby analyzing the proteomics sample.

Differential Lableing and Quantitation of Members of a Biologically-Derived Sample

Various techniques for the analysis of pairs of samples, e.g., based on chemical labeling for assessing quantitative differential display of base proteins or PTMS, have been described in the art. The chemical labels used in these techniques are typically prepared from two differing components that performing separate and independent functions (three in the case of differential quantitation). The first portion directs the chemical labeling of functional groups in the peptides of interest, while the second portion enables the selective isolation of the labeled peptides. Examples of such chemical labels include those employed in the isotope-coded affinity tagging (ICAT) methodology (Gygi et al., “Quantitative analysis of complex protein mixtures using isotope-coded affinity tags” (1999) Nature Biotechnology 10:994-999), as well as thiol-type Michael addition reagents used for serine and threonine phosphorylation analysis (see, for example, Goshe et al., supra).

These methods for the simplification of complex mixtures and/or concentration of specific protein classes of interest have proven critical in the thorough analyses of target molecules. However, these commonly-employed methodologies have several operational problems, including a) the presence of nonspecific interactions, b) difficulty in fully recovering the species of interest from high specificity binding systems, and c) unwanted fragmentation of the label during analytical processes such as tandem MS, thereby decreasing the effectiveness of tandem MS processes.

The present invention overcomes these and other problems in the art by providing new methods and compositions that take advantage of fluorous properties. Differential labeling capability can also easily be built into these systems. For example, many of the fluorous labeling reagents provided in Table 2 includes a short linker element e.g., —CH₂CH₂— or —CH₂CH₂CH₂—, between the fluorous moiety and the chemically-reactive functional group. For reagents such as the thiol-type fluorous labeling reagents, this linker element assists the thiol in retaining its nucleophilicity. For differential labeling embodiments, deuterated and normal versions of the alkyl chain are employed, enabling differential quantitation (see, for example, the iodoacetamide-type fluorous labeling reagents 6a through 6d).

It should be noted that the length of both the alkyl and the perfluoroalkyl chain can also be varied as desired. Species with perfluoroalkyl chains of different chain length can be separated from each other with specific retention properties that persist regardless of the nature of the bound species. In theory, this property could be used to multiplex separations from different samples simultaneously, a process that would be extremely difficult to do by other methodologies. Further, the data arising from having different length chains (that clearly have different masses) attached to different labeling reagents (that would label different amino acids/functional groups) could be used to elucidate amino acid composition prior to tandem MS analysis. Optionally, these parameters can also be programmed into the tandem MS software analysis program, giving rise to further confidence in peptide identifications.

The present invention provides methods for differential quantitation using the compounds and derivatives disclosed herein. In this aspect, samples to be compared are reacted with different isotopic versions of the same reagent, and the two derivatized samples are combined. The result is a series of isotopically labeled polypeptide pairs, with the relative concentration of each member of a given pair being directly proportional to its signal intensity. The isotopic substitutions can exist within the fluorous moiety itself in the form of ¹³C atoms, within the linker-region as a variety of stable isotopes, or as part of the amino acid conjugation agent itself. Alternatively, the isotopic substitutions can be incorporated such that they remain with the isolated peptide if a cleavable reagent is employed Advantageously, the methods of the present invention provide differential quantitation while simultaneously maintaining the label's other desirable properties.

An exemplary pair of isotopic reagents includes, but is not limited to, tridecafluorooctyl acrylate and its 3,4,5,6,7,8-¹³C₆ tridecefluorooctyl analog. Protein samples (1 mg) to be compared are reduced with TCEP and digested with trypsin. The digests are desalted, dried, and reconstituted in 200 μL dimethyl formamide (DMF), and 2.5 μL 100 mM sodium carbonate, pH 8.0. 1 μL of tridecafluorooctyl acrylate or its ¹³C₆ are added to each sample individually, and the reactions are allowed to proceed overnight at room temperature. The samples are combined, and unreacted acrylates are removed from the mixture by incubation with 4 mg N-2-mercaptoethylaminomethyl polystyrene beads (Nova Biochem) at room temperature for 2 hours. FSPE is performed to isolate only the fluorous labeled (and thus cysteine-containing) peptides, each of which exists as an isotopic pair separated by 6 Daltons per cysteine moiety, and the relative concentration between the two samples is reflected in the pair's signal intensities.

Separation by Fluorous Content

In a further aspect, the present invention provides methods for separation of components of a biologically derived (e.g., proteomics or metabolomics) sample based upon the fluorous content of the labeled species. A biologically-derived sample, such as a proteomics or metabolomics sample having a plurality of amino acid-containing components (proteins, peptides, and the like), is treated with a fluorous labeling reagent as described herein (e.g., having a chemically-reactive functional group coupled to a fluorous moiety having five or more fluorine atoms). Member components of the sample are labeled with the fluorous labeling reagent, such that some member components are coupled to a single fluorous label while other member components are coupled to more than one fluorous label. The sample members can then be fractionated according to fluorous content; the treated sample is combining with a fluorous separation composition (e.g., a fluorous affinity matrix) to allow selective elution of bound single labeled components separately from bound multiply labeled components.

For example, the methods of the present invention can be used for the analysis of proteomics or metabolomics samples having ubiquitinated components. Ubiquitin is a highly conserved polypeptide that, once coupled (via a lysine residue) to a cellular protein, tags that protein for degradation. As such, a ubiquitinated protein has two N-termini, one from the primary sequence of the protein itself and one from the coupled ubiquitin moiety. This property can be put to use for the analysis of a sample containing ubiquitinated components.

The sample can be considered as having two portions, a first portion consisting of those proteins having a single N-terminal residue, and a second portion including ubiquitinated proteins having at least two N-terminal residues (one derived from the attached ubiquitin sequence). Appropriate cleavage of the sample members into fragments (e.g., by trypsin) will thus generate two populations of peptides, one portion having the single N-terminus, and the second portion (i.e., the peptides containing the ubiquitinated lysine residue) having two N-termini. In a preferred embodiment o the methods, the epsilon-amino groups of any unmodified lysine residues are blocked (e.g., via guanidination) prior to treating the sample with a fluorous labeling reagent that targets amino groups.

Once the non-terminal amino groups are blocked, labeling the first and second N-termini of the proteolytic fragments with the fluorous labeling reagent produces a first portion of single-labeled proteolytic fragments and a second portion of multiply-labeled (e.g., dual-labeled) proteolytic fragments.

In a similar manner, intermolecular disulfide-linked peptides also effectively have two N-termini, one from each peptide. The methods of the present invention can be used for the analysis of proteomics samples having intermolecular disulfide-linked components. Treating the proteomics sample having one or more disulfide-bonded components includes the steps of cleaving the disulfide bridge-containing components with trypsin, thereby generating one or more disulfide-linked proteolytic fragments having two N-termini; and labeling the N-termini of the proteolytic fragments.

Multiplexing Using Multiple Fluorous Tags

In yet another aspect, the present invention provides additional methods of multiplex separation of a proteomics sample using fluorous-based separation techniques. The multiplex separation is performed on a set of biologically-derived samples (i.e., two or more proteomics or metabolomics samples, each member sample having a plurality of components), using two or more fluorous labeling reagents. Typically, the fluorous labeling reagents are amino acid conjugation agents coupled to fluorous moieties having five or more fluorine atoms. A first member of the set of samples (i.e., a first plurality of components) is treated with a first fluorous labeling reagent, thereby labeling one or more components (e.g., proteins, peptides, and the like) in the first sample. In a similar manner, a second member of the set is treated with a second (different) fluorous labeling reagent, thereby labeling one or more components of the second sample. Optionally, additional sample sets can be labeled using additional fluorous labeling reagents. The first, second, and any additional fluorous labeling reagents optionally employed in the methods have different chemical structures; preferably, the fluorous labeling reagents differ in the number of fluorine atoms incorporated therein.

The treated first and second samples are combined, to form a combined sample. A fluorous-based separation technique is then used to separate labeled and non-labeled components in the combined sample. In some embodiments, a fluorous solid phase extraction is performed, to separate the labeled and unlabeled species. The differentially labeled species can optionally be further fractionated, or they can be analyzed together. Alternatively, fluorous column chromatography using an affinity matrix can be used to separate the labeled and non-labeled components, as well as for separating the components labeled with the first fluorous labeling reagent from the components labeled with the second fluorous labeling reagent. Optionally, the method further includes the step of analyzing the non-labeled components or the fluorous labeled components, e.g., by mass spectrometry.

As noted above, one or more additional members of the set of proteomics samples can optionally be treated with additional fluorous labeling reagents, which additional reagents differ from each other and from the first and second fluorous labeling reagents (e.g., in the number of fluorine atoms incorporated therein). These additional treated samples can also be combined with the first and second samples prior to the separating step. The analysis can be performed by mass spectrometry, e.g., as noted for the previous methods.

Differential Quantification Reagents

The present invention also provides sets of fluorous labeling reagents for differential quantification of a proteomics or metabolomics sample. A set of fluorous labeling reagents typically includes two or more fluorous labeling reagents as described herein, which are differentially labeled with one or more stable isotopes. While various stable isotopes can be employed, the isotopes more commonly used in the set of fluorous labeling reagents are deuterium (²H), carbon-13 (¹³C), nitrogen-15 (¹⁵N), and oxygen-18 (¹⁸O). The difference in isotope between a pair of fluorous labeling reagents can be positioned, e.g., in the fluorous moiety (e.g., a ¹³C-perfluoroalkyl group), in a retained portion of the chemically-reactive functional group, or in an optional linker region coupling the fluorous and conjugation moieties. Exemplary pairs of fluorous labeling reagents that can be employed in differential quantification analysis are compounds 6a and 6b, and 6c and 6d.

Kits

In an additional aspect, the present invention provides kits embodying the compositions and/or methods provided herein. Kits of the invention optionally comprise one or more of the following: (1) one or more fluorous labeling reagents as described herein; (2) a fluorous matrix or other materials for performing fluorous solid phase extractions; (3) instructions for practicing the methods described herein, and/or for using the fluorous labeling reagents described herein; (4) one or more biologically-derived sample components (e.g., for use as control(s) during analysis); (5) a container for holding components or compositions, and, (6) packaging materials.

### EXAMPLES

The present invention provides various aspects of fluorous labeling of proteomics and metabolomics sample constituents, including the description of unique reagents not described previously, as well as examples of their usage in the preparation and isolation of a variety of functional species from more complex mixtures. The following examples are offered to illustrate, but not to limit the claimed invention. For example, although only one fluorous labeling reagent may be specified in a given reaction scheme or example, similar labeling reagents of that type of reagent (for example, differing in chain lengths or number of incorporated fluorines) are implied. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Fluorous Solid Phase Extraction (FSPE)

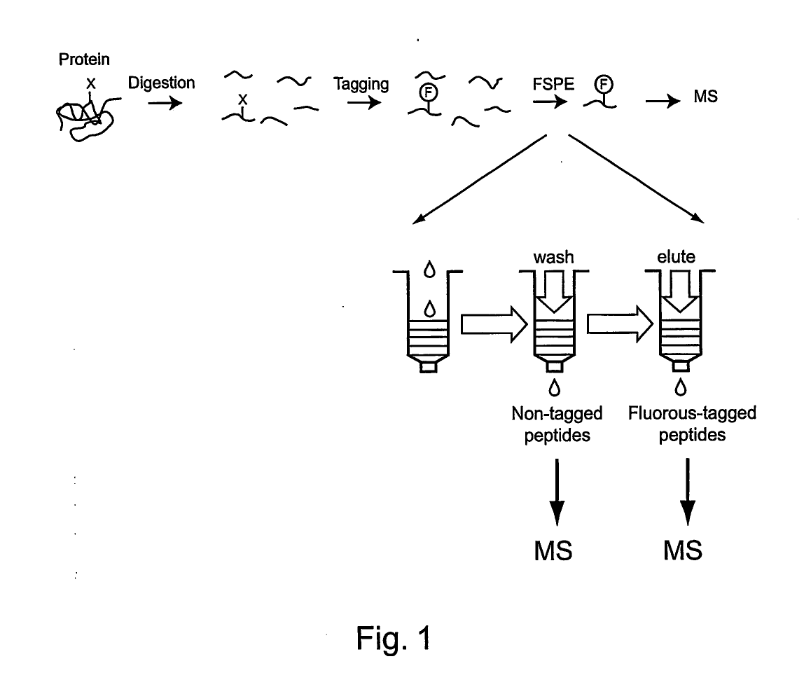

Fluorous columns for use in fluorous solid phase extraction procedures, such as that depicted in FIG. 1, can be prepared as follows. Fused silica capillaries (360/200 μm O.D/I.D, approximately 15 cm in length) were first ‘Kasil’ flitted and then packed under high pressure (500-1000 psi, using an in-house built pressure vessel) with a slurry of Fluoroflash™ fluorous reversed-phase silica gel (FRPSG, perfluorooctane bonded phase, 5 μm particles, available from Fluorous Technologies, Inc., Pittsburgh, Pa.) to a total bed length of approximately 5-8 cm. The slurry was prepared by adding a spatula tip of FRPSG to 500 μL MeOH with magnetic stirring. ‘Kasil’ material was prepared by mixing 450 μL Kasil No. 1 potassium silicate (The PQ Corp., Valley Forge, Pa.) with 88 μL formamide, followed by vortex mixing. The mixture was centrifuged for 1 min (˜5000 rpm) and the top 200 μL removed and saved. The fused silica capillaries were then quickly dipped into the saved solution, allowing 1-2 cm of the material to enter one end of the capillary. The ‘dipped’ capillaries were baked at 100° C. for 1 hour, allowed to cool, and trimmed with a ceramic cutter to a Kasil frit length of approximately 1-2 mm, and a total capillary length of approximately 10 cm. Finally, the fritted capillaries were packed with the above-mentioned FRPSG slurry, and activated with 20-50 column volumes (CV) of 99% methanol/10 mM ammonium formate.

Following equilibration with 20-50 CVs of 60% methanol/10 mM ammonium formate, samples were loaded onto the FRPSG column in the equilibration buffer. Subsequently, a wash step was performed with 20-50 CVs of either wash A (60% methanol/10 mM ammonium formate) or wash B (50% acetonitrile in water v/v) depending on the fluorous tag employed (C₆F₁₃ tag=wash A, C₈F₁₇ or 2×C₄F₉ tag(s)=wash B). Fluorous-tagged peptides were eluted using 20-50 CVs of 99% methanol/10 mM ammonium formate. Fractions were collected into 0.5 mL microcentrifuge tubes, and subsequently dried in vacuo. The dried FSPE eluent (99% methanol/10 mM ammonium formate fraction) was reconstituted in 25% methanol/0.5% acetic acid (v/v) for further analysis, e.g., by MAIDI-TOF MS or capillary LC/MS.

### Example 2

### Guanidination and α-Amino Fluorous Derivatization of Tryptic Peptide Mixtures

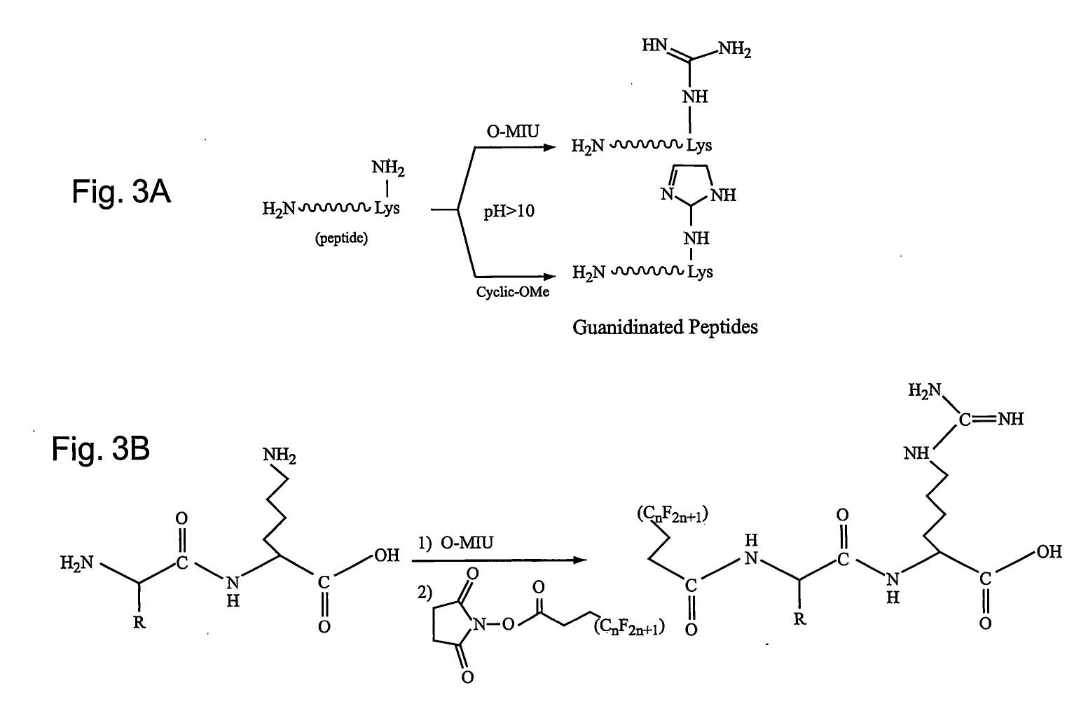

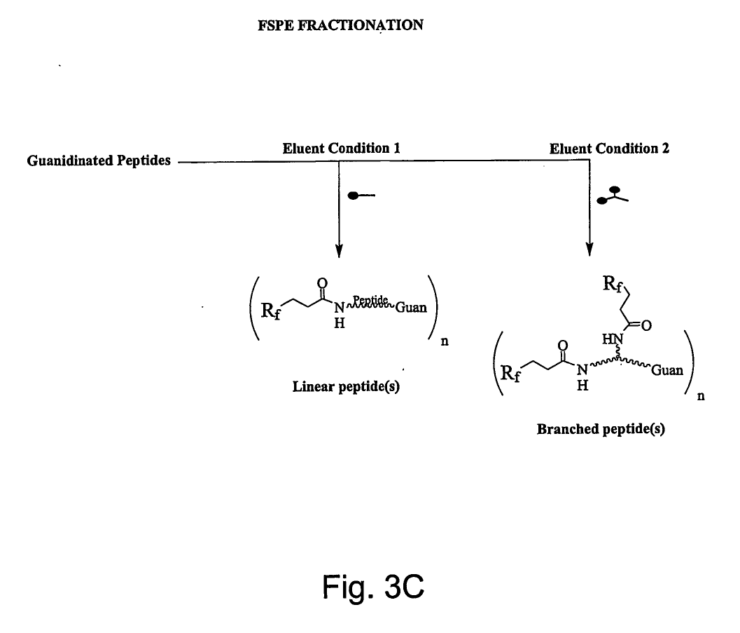

Selective reaction of a lysine e-amino group using either O-methylisourea or 2-methoxy-4,5-dihydro-1H-imidazole is depicted in FIG. 3A. Typically, the lysine residues were modified after capture on μC₁₈ Ziptips™ as described herein. The tips were aspirated with the respective solutions several times and incubated in a 50° C. oven with the tip bed fully immersed in reactant solution.

Lysine conversion to homoarginine was performed during a 2 hour incubation at 37° C., using a 1:4 solution of 0.5 M O-methylisourea hydrogen sulfate (2 μL) and 0.25 M sodium carbonate, pH 11.7 (8 μL), or using O-methylisourea hemisulfate (O-MIU, ˜1.1M in 0.25M sodium bicarbonate pH 10.5). The modified peptides were then desalted and dried in vacuo.

Conversion of lysine t-amino group(s) to the 4,5-dihydro-1H-imidazoyl derivative was performed using 2-methoxy-4,5-dihydro-1H-imidazole (Cyclic-OMe, ˜0.8M in water), and incubation for 4-5 h (or as described in the Lys Tag 4H reagent kit available from Agilent Technologies, Wilmington, Del.).

N-terminal amino groups (on either lysine blocked or untreated samples) were fluorous labeled by addition of an equal volume of 0.25 M sodium bicarbonate buffer and freshly prepared 250 mM N-succinimidyl 3-perfluorobutyl propionate (compound 5a) in THF, for a final total volume of 40 μL. The labeling reaction proceeded for 2 h at room temperature, followed by addition of 4 μL aq. 50% hydroxylamine solution (to reverse unwanted esterifications of tyrosine and histidine residues). The reaction solution was allowed to stand for 10 min, after which 5 μL of 5% TFA was added to terminate the reaction. Finally, the reaction solution was dried in vacuo, then reconstituted in 60% methanol/10 mM ammonium formate.

### Example 3

### Fluorous Labeling of Phosphorylated or Glycosylated Peptides Via β-Elimination and Thiol Michael Addition

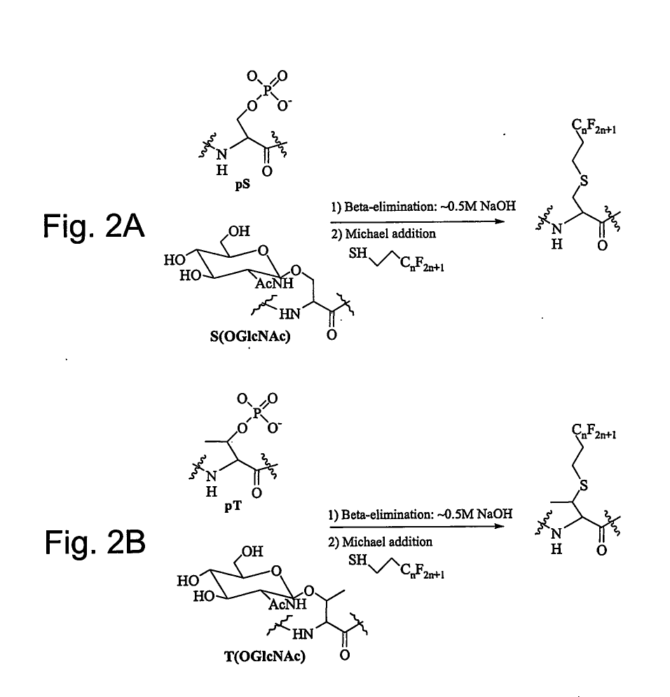

Exemplary reaction schemes depicting the β-elimination and subsequent labeling of phosphorylated and/or glycosylated serine or threonine residues via a Michael addition using a thiol-type fluorous labeling reagents is provided in FIGS. 2A and 2B.

Bovine α-casein and chicken ovalbumin were purchased from Sigma-Aldrich (St. Louis, Mo.). Unless otherwise noted, protein samples (˜40 μM) were reduced by the addition of 5 mM dithiothreitol (DTT) in 50 mM ammonium bicarbonate (pH 7.5). Proteolysis with sequence grade modified trypsin (Tp) (Promega, Madison, Wis.) was carried out overnight at 37° C. using a substrate/enzyme ratio of 50:1 (w/w).

Samples (5 μL,≦250 pmol) were combined with an equal volume of 3:1 DMSO/ethanol (v/v) (5 μL), followed by the addition of 4.6 μL saturated Ba(OH)₂ and 1 μL 500 mM NaOH. Finally, 0.7 μL of 1H,1H,2H,2H-perfluorodecane-1-thiol (fluorous labeling reagent 1a) was added and the solution allowed to react at 37 ° C. for 1 h. Reactions were stopped by addition of 5 μL 5% TFA (v/v), and the reaction products subsequently oxidized by adjusting the reaction mixture to a final concentration of 3% H₂O₂ (v/v), and allowing the reaction to occur for 30 minutes at RT. This results in fluorous labeled sample members having a β-linked fluorous sulfoxide side chain in lieu of the former phosphoserine (pS) or phosphothreonine (pT) residues. Finally, the samples were diluted to 100 μL with 60% methanol (v/v)/10 mM ammonium formate and stored at −80° C.

In an alternative small scale β-elimination/Michael addition reaction (≦100 pmol peptide), 11.35 μL aqueous peptide solution was mixed with 5.11 μL ethanol, 1.84 μL 5M NaOH, and 1.18M of 1.7 μL of 1H,1H,2H,2H-perfluorodecane-1-thiol(fluorous labeling reagent 1a) in dimethylformamide. The reaction was allowed to proceed at room temperature for 2-3 h.

Furthermore, optionally extending the oxidation process will convert the β-linked fluorous sulfoxide side chains of labeled peptides to their fluorous sulfone analogs, which exhibit improved tandem mass spectrometry fragmentation properties.

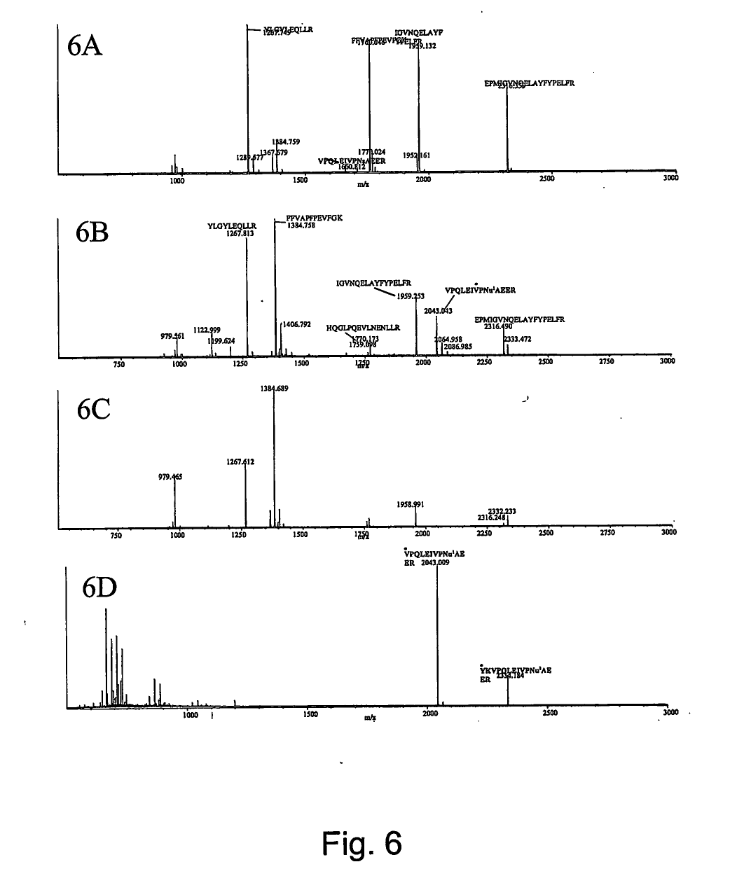

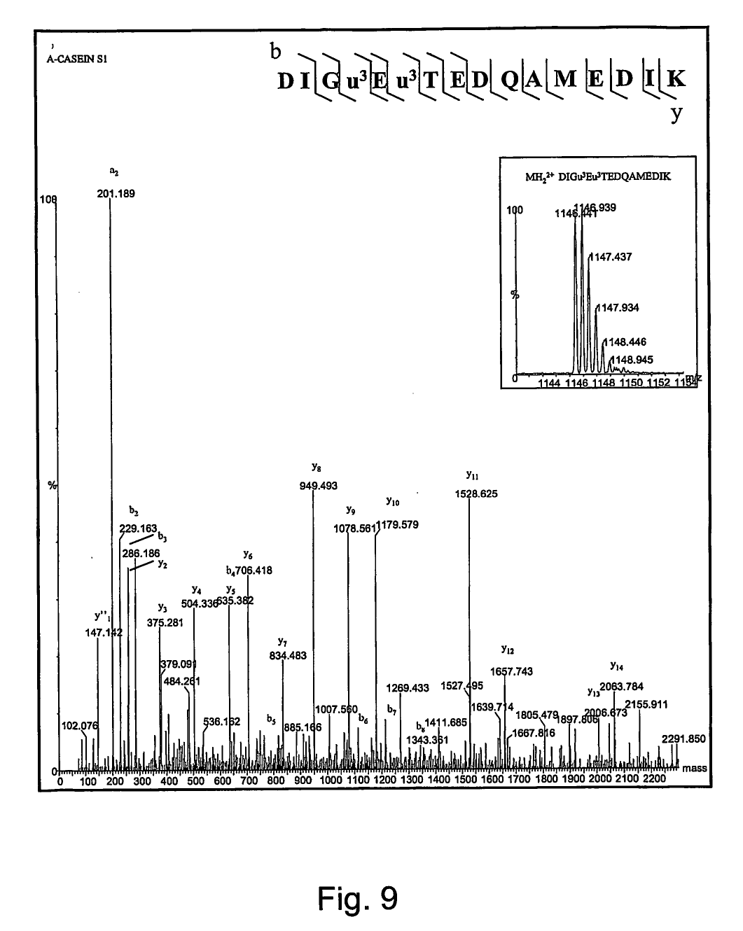

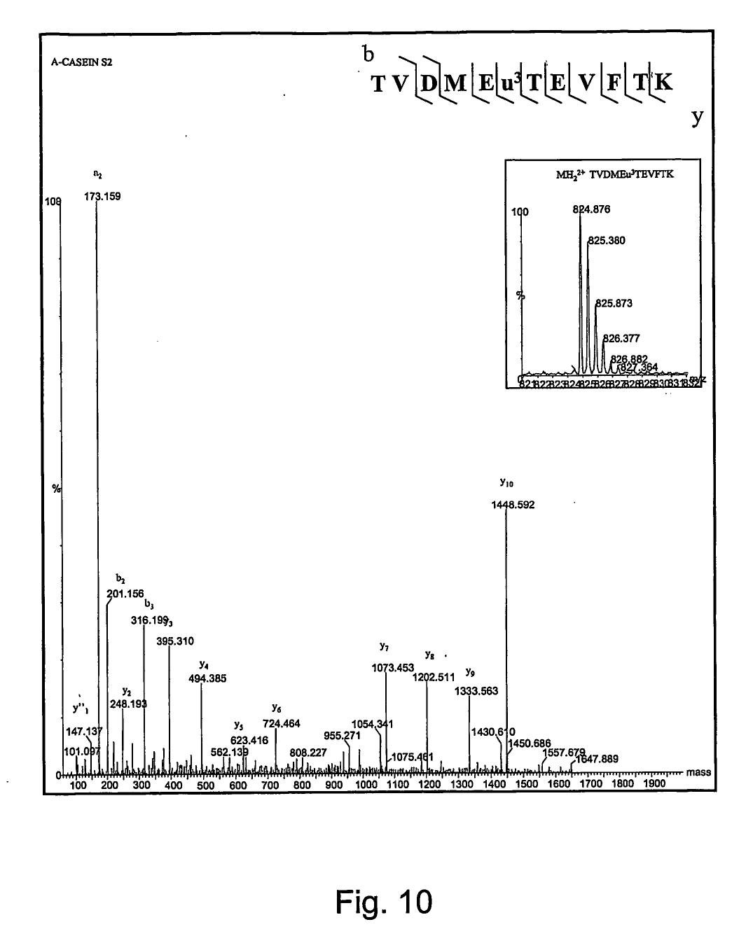

This reaction scheme can be used to label a number of β-elimination products derived from a biologically-derived sample. FIGS. 6-11 provide experimental data generated upon fluorous labeling of α-casein digests (using fluorous labeling reagents CF₃(CF₂)₇CH₂CH₂SH, and 1H,1H,2H,2H-perfluorohexane-1-thiol). The upper panels in FIG. 6 provides MS data generated for the (unlabeled) digested casein sample prior to (first panel) and after (second panel) undergoing the β-elimination reaction and fluorous labeling. The lower panels depict sample contents that were either retained (fourth panel) or not retained (third panel) upon separation with a fluorous affinity matrix. FIGS. 7-10 depict tandem MS data generated for various identified peptide fragments. The spectra also provide an example of the inertness of the fluorous labeling reagents under tandem MS conditions; two identical species with different sized tags produce spectra that are identical except for mass shifts due to the different labels.

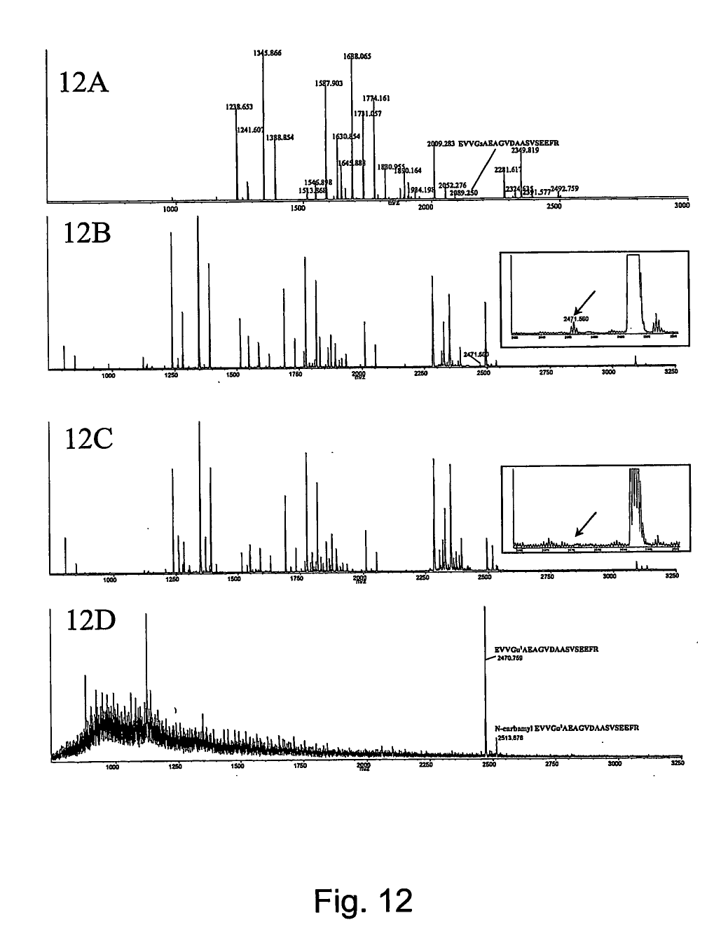

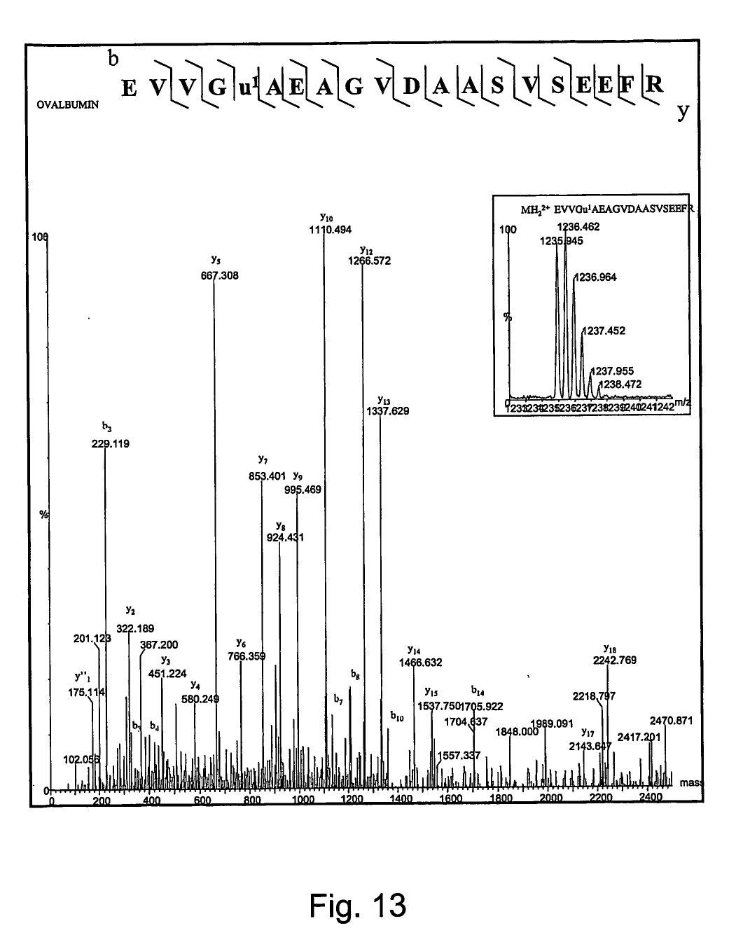

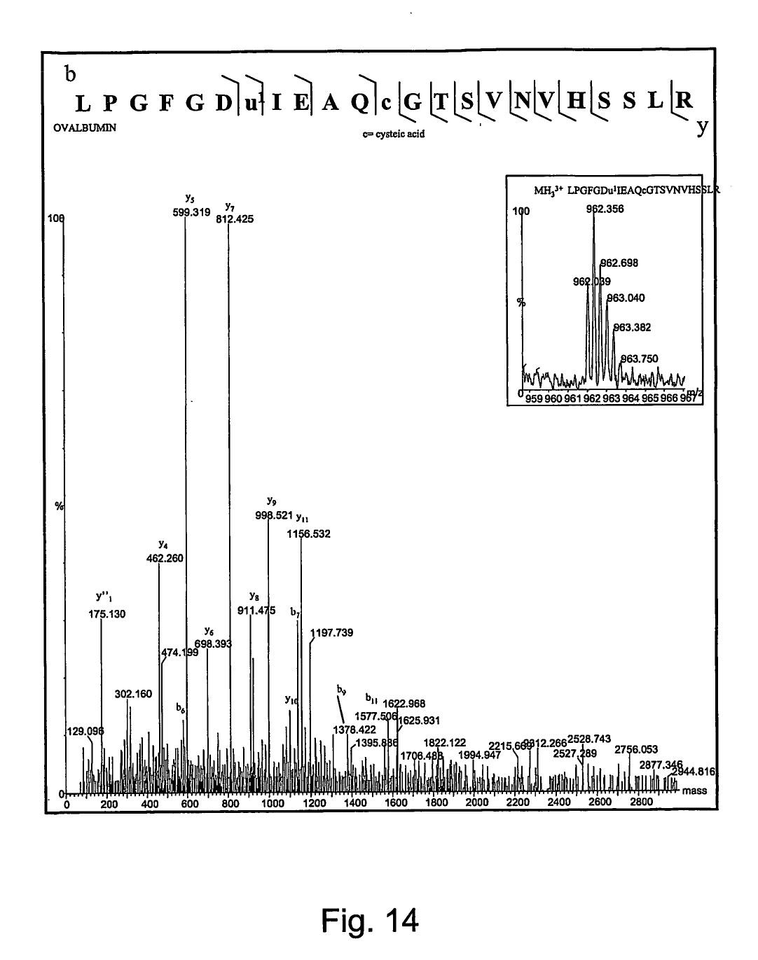

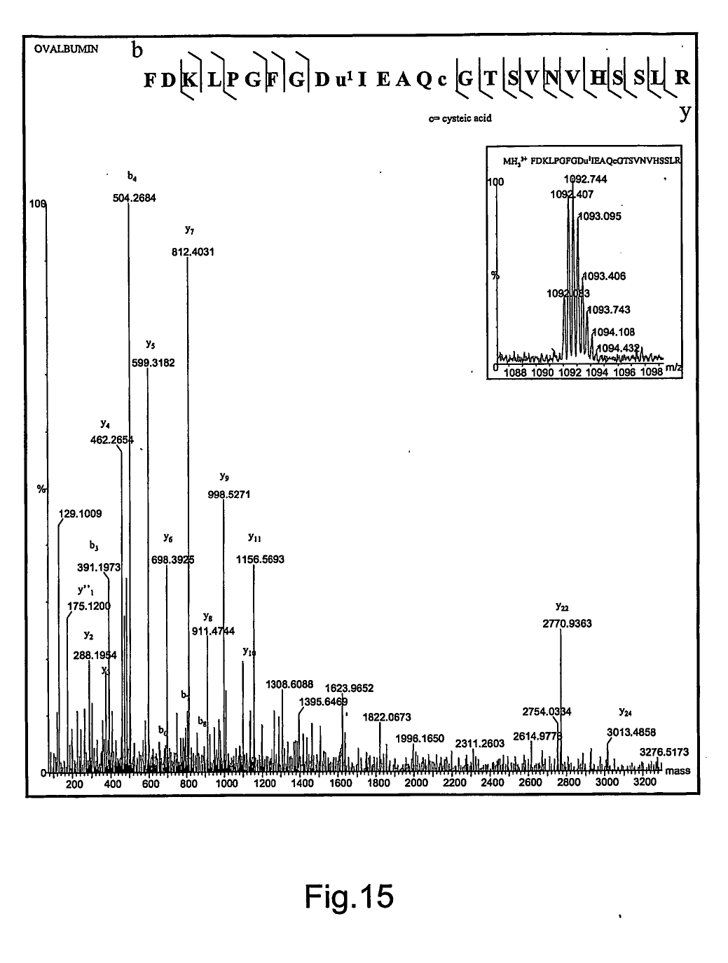

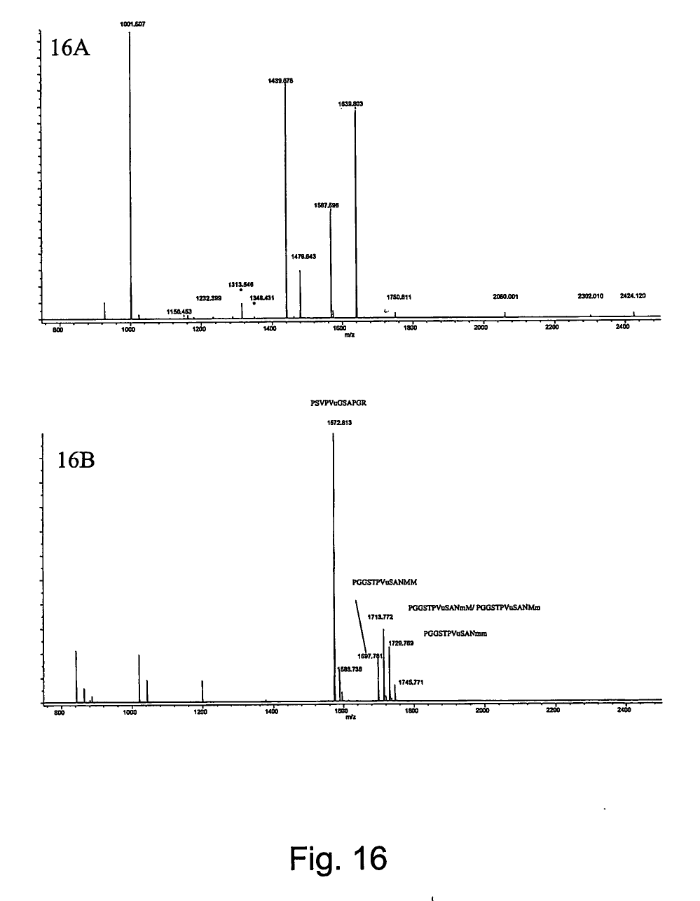

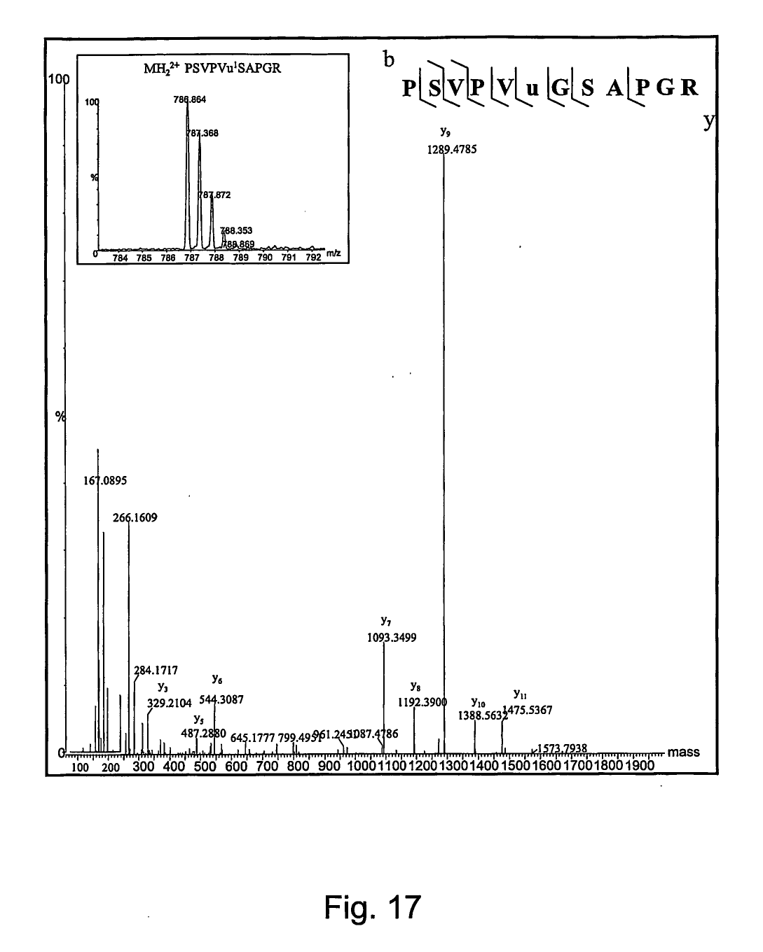

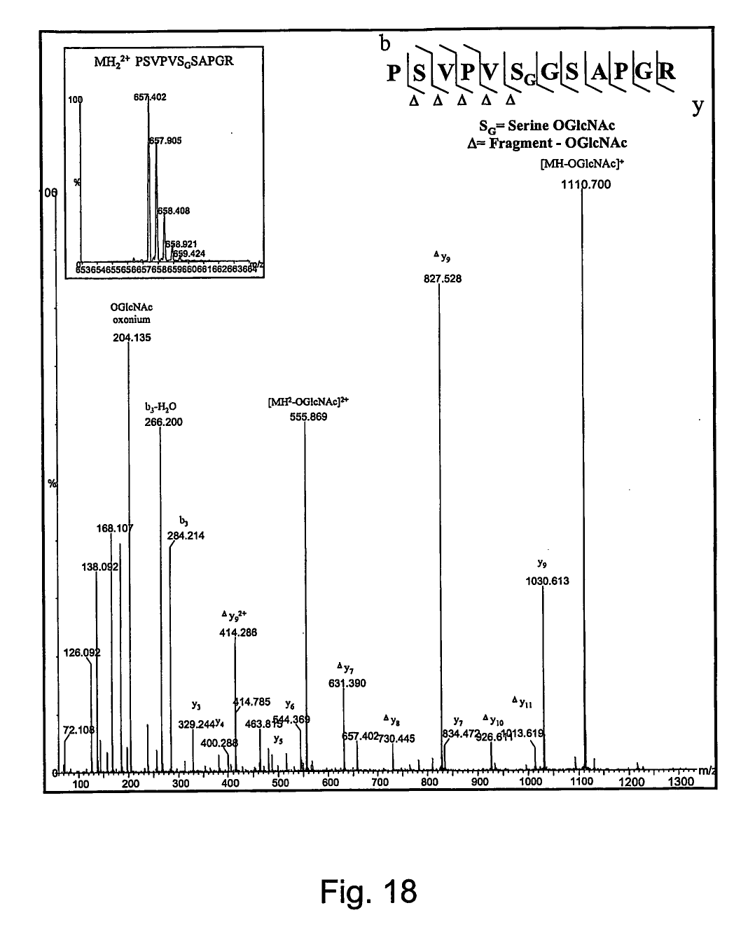

FIGS. 12-15 provide results obtained from analogous experiments performed using the protein ovalbumin, including MS data showing the alterations in MS peak positions after tryptic digestion and fluorous labeling of the phosphopeptides, as well as the corresponding tandem MS data. In a further set of experiments, O-GlcNAc peptides were fluorous labeled in a similar ′-elimination/Michael addition reaction, as depicted in FIGS. 16-18.

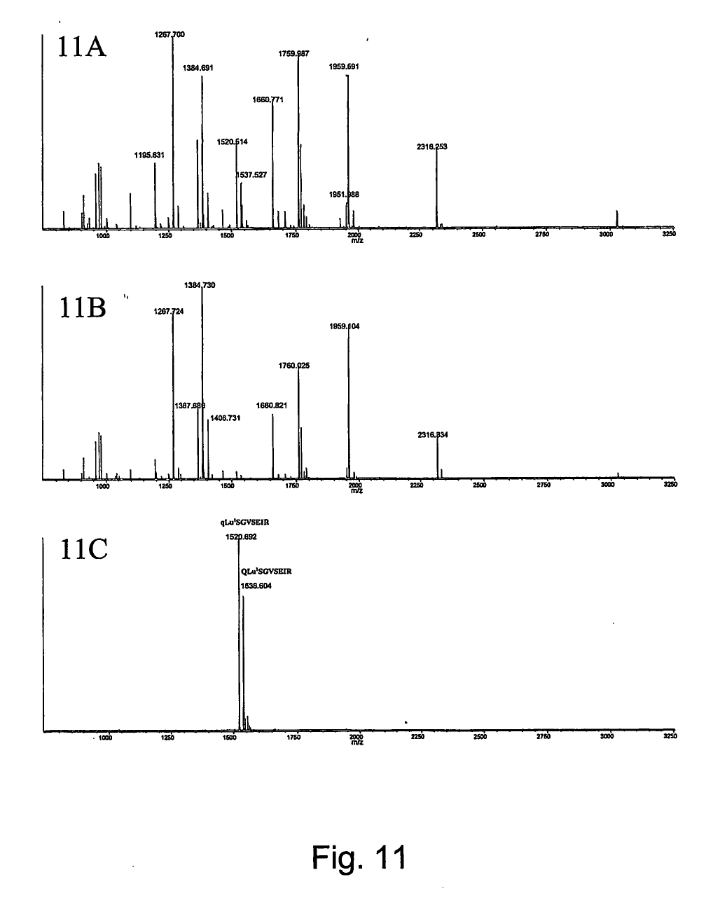

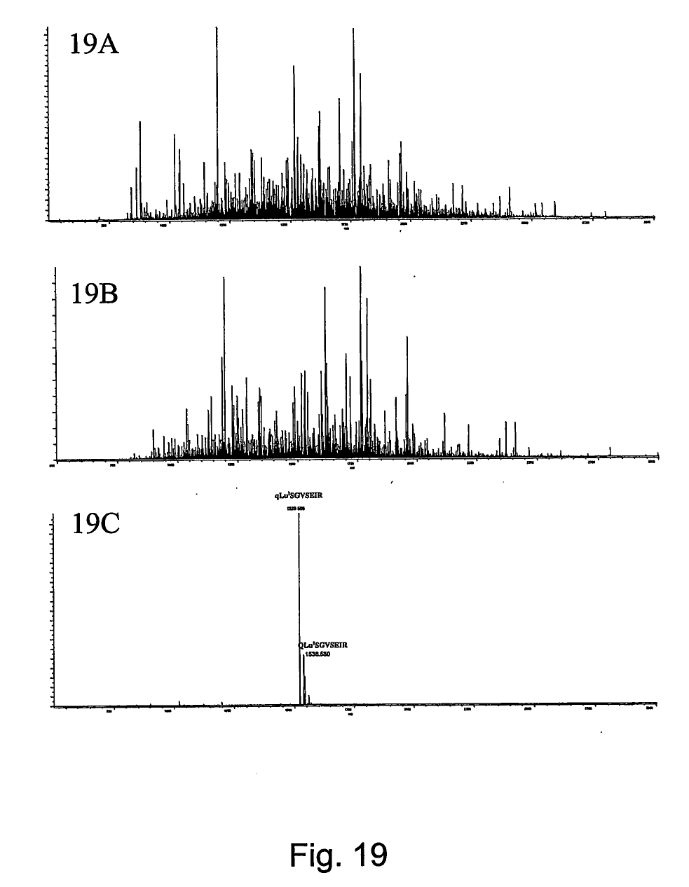

In a related embodiment, fluorous labeled phosphopeptides were prepared by a similar β-elimination/Michael addition reaction, and then used to “spike” tryptic digests of unlabeled casein (as shown in FIGS. 11). FIG. 19 also depicts data generated for samples prepared by spiking of β-elimination labeled phosphopeptides into a whole yeast tryptic digest. Both experiments demonstrate the highly specific retention characteristics of the fluorous label(s) as compared to unlabeled species.

### Example 4

### Fluorous Labeling of Cysteinyl Peptides via Acrylate Michael Addition

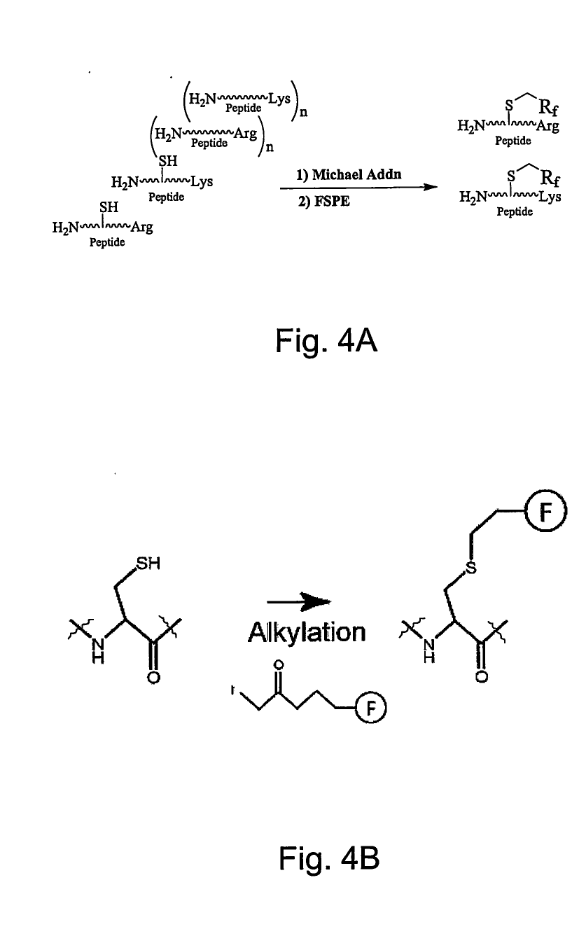

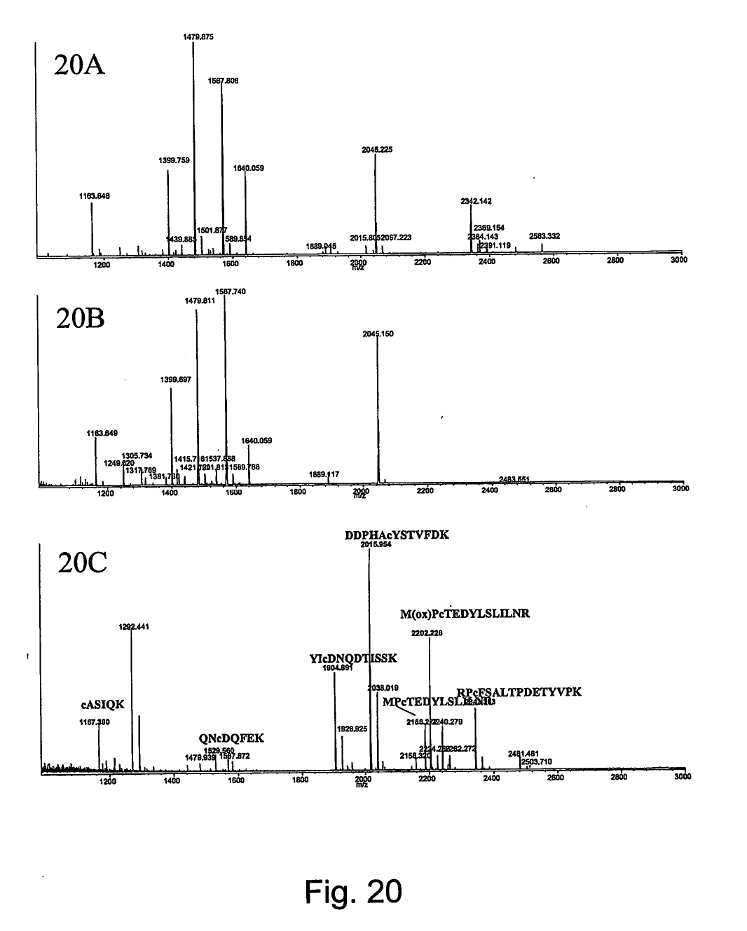

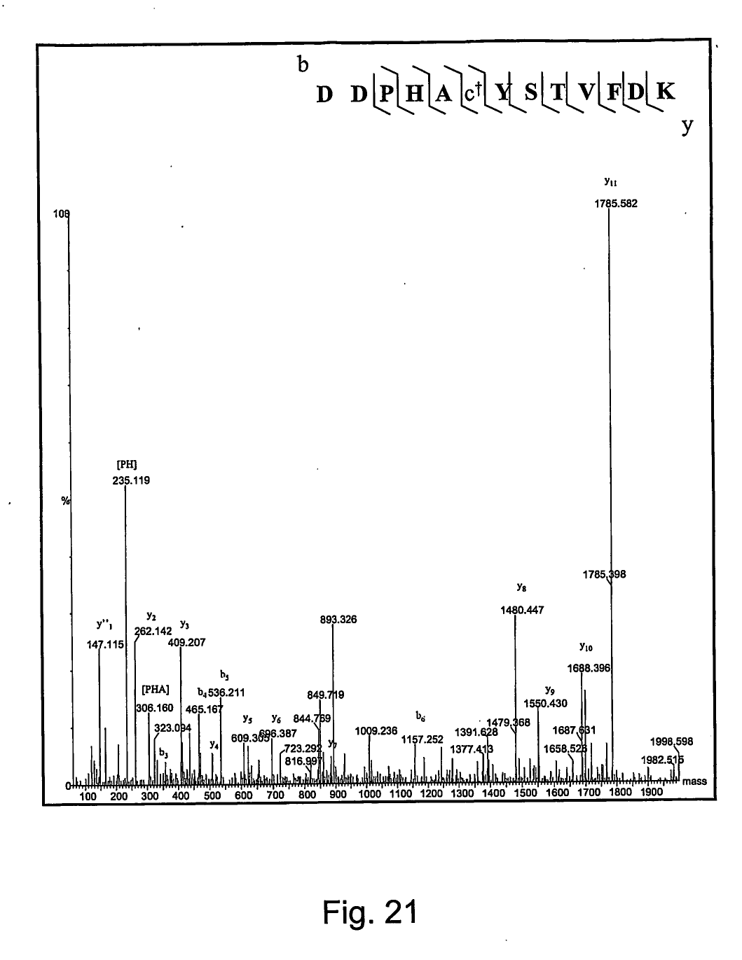

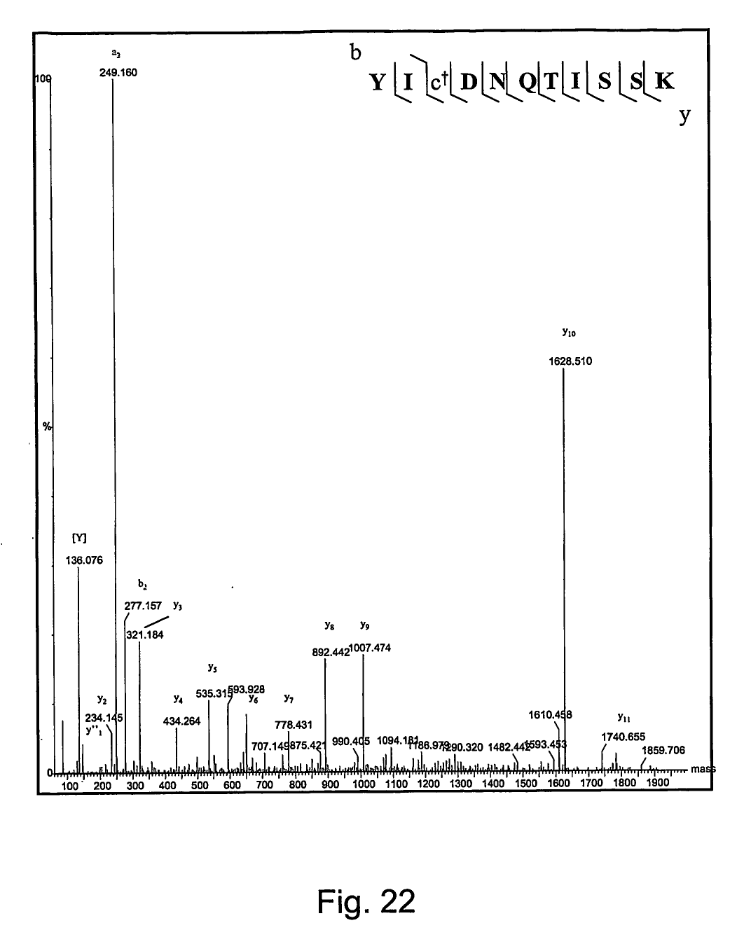

An exemplary reaction scheme depicting an Michael addition of cysteinyl peptides from BSA to an acrylate fluorous labeling reagent is provided in FIG. 4A. Exemplary data generated using this reaction scheme include the comparative MS profiles depicted in FIG. 20, and the tandem MS data of labeled peptides are provided in FIG. 21 and FIG. 22.

Bovine serum albumin (SA, 1 mg) was dissolved in 100 μL of 4M Urea, 0.1 M ammonium bicarbonate, pH 8.0. Triscarboxyethylphosphine (TCEP) in water was added to a final concentration of 10 mM, and the mixture was allowed to stand for 10 minutes at room temperature. Tp (20 μg) was added, and the mixture incubated at 37° C. for 8 hr. An aliquot corresponding to 1 nmol tryptic peptides was loaded onto a Peptide Macrotrap (Michrom Bioresources, Auburn, Calif.). The desalting column was rinsed with 1 mL of 0.1% acetic acid (v/v), and peptides were eluted with 70% acetonitrile/0.1% acetic acid (v/v). This mixture was evaporated to dryness. in vacuo.

The tryptic peptides were reconstituted with 200 μL dimethyl formamide (DMF), 2.5 μL 100 mM sodium carbonate, pH 8.0, and 1 μL 3,3,4,4,5,5,6,6,7,7,8,8,8-tridecafluorooctyl acrylate 4a (TDFOA, 3.7 μmol). The reaction was allowed to proceed overnight at room temperature (RT), during which the free thiol groups of cysteine were coupled with the TDFOA via a Michael-type addition. Unreacted TDFOA was removed by incubation with 2 mg N-2-mercaptoethylaminomethyl polystyrene beads (Novabiochem,) at RT for 2 h. After the reaction period, the supernatant was removed and diluted for further processing and/or analysis.

A similar reaction was performed using with a C₈F₁₇ analog of TDFOA. While the C₆₁₃ species was more amenable to direct LC/MS analysis, the C₈F₁₇ version performed better with respect to isolation of the labeled species (“on-off” capture) and MALDI analysis.

### Example 5

### Fluorous Labeling of Cysteinyl Peptides via Alkylation

Alternatively, a fluorous iodoacetamide alkylating agent (N-[(3-perfluorooctyl)-propyl]-iodoacetamide (compound 6a) was employed as a thiol-targeting fluorous labeling reagent. BSA was reduced with immobilized TCEP, digested with Tp for 8 h at 37° C., and desalted as described previously. 100 pmol of the tryptic digest in 20 μL water was mixed with 5 μL 1M ammonium bicarbonate, 20 μL THF, and 5 μL 500 mM N-[(3-perfluorooctyl)-propyl]iodoacetamide (500 mM stock solution in THF) and allowed to react for 30 minutes at 37° C. in the dark. After allowing to cool, 450 μL 50% methanol/50 mM ammonium bicarbonate was added and mixed. Excess fluorous iodoacetamide was removed by addition of several mg of 3-mercaptopropyl functionalized silica gel (Aldrich, Milwaukee, Wis.), followed by agitation for several hours in the dark at room temperature. The slurry was then filtered through a 3K cellulose molecular weight cut-off filter, followed by a methanol wash (100 μL) of the filtered silica gel. The combined filtrate and wash was then dried in vacuo, and reconstituted in 60% methanol/10 mM ammonium formate.

This and other examples described herein optionally employ solid-phase scavenging agents to remove the excess reagent. One of skill in the art would recognize that the nature of the resin (polymeric vs. silica), as well as the rinsing conditions employed, will influence the effectiveness of this methodology, and will be able to optimize these parameters without undue experimentation. Alternatively, reagent removal schemes not involving solid-phase scavenging could also be applied.

### Example 6

### NHS-Ester Amidation of Primary Amines

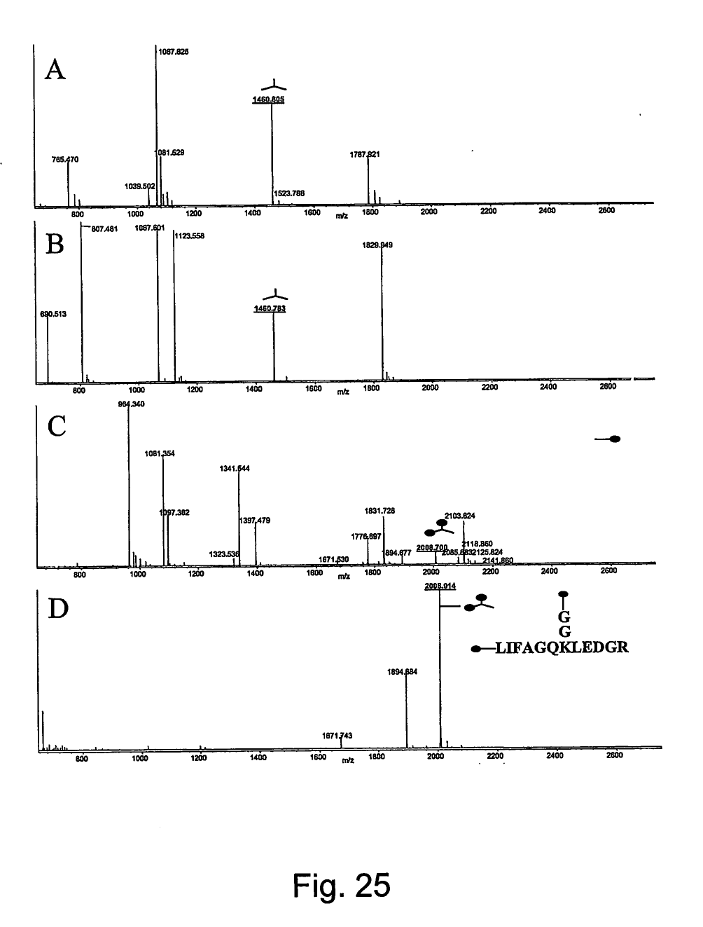

An exemplary reaction scheme depicting synthesis of an NHS-ester type fluorous labeling reagent (and amidation of primary amines using this reagent) is provided in FIG. 3B. In addition to single labeling of peptides (e.g., N-terminal labeling of “linear” peptides), in some embodiments of the present invention, this labeling reaction scheme is used for double labeling of proteomics sample members (for example, in the case of “branched” peptides, such as those formed by ubiquitination or intermolecular disulfide reactions). A schematic representation of linear and branched labeled peptides is depicted in FIG. 3C, while comparative MS profiles (panels A: untreated, B: fluorous-labeled, C: non-retained, and D: species retained by a fluorous affinity matrix) are provided in FIG. 25. In these experiments, the polyubiquitin chains (Affiniti Research Products, Exeter, UK) were digested with trypsin in a solution of 100 mM ammonium bicarbonate, 4 M urea, pH 8.0.

N-hydroxysuccinimidyl -2H,2H,3H,3H-perfluoroheptanoate was synthesized similarly to the method of Hall et. al. (2003 J. Mass Spec. 38:809) by adding 17.2 mg of 2H,2H,3H,3H perfluoroheptanoic acid, 14.6 mg N-hydroxysuccinimide, and 20.2 mg ethyldiethylaminopropylcarbodiimide (EDC) to 500 μL DMF. Desalted peptides (ca. 6 μL) in 70% acetonitrile, 0.1% TFA were added to a mixture of 10 μL Na₂HPO₄, pH 8.0, 10 μL DMF, and 1 μL (ca. 100 nmol) of the PFHA NHS ester.

This reaction scheme can be used for single amine labeling, as well as multiple amine labeling (e.g., of branched peptides). Furthermore, as noted herein, structurally-related families of such reagents having different chain lengths can be employed. In addition, bifunctional crosslinkers such as those provided in Table 2 can also be used. Longer chain species can be kept more aqueous compatible by making the corresponding sulfoNHS ester derivatives.

### Example 7

### Preparation of Yeast and Jurkat Whole Cell Protein Fractions for Alkaline β-Elimination/Fluorous Michael Addition

Yeast cake (S. cerevisiae) was purchased from a bakery supply store and subsequently pulverized under liquid N₂ and stored at −80° C. Cellular protein was isolated using Trizol™ reagent (Invitrogen, Carlsbad, Calif.), and subsequently oxidized by incubation overnight at 4° C. in 50 μL oxidation solution (e.g. 4.5 mL 88% formic acid and 0.5 mL 30% H₂O₂ solution that was first allowed to sit at room temperature UT) for 2 h, then stored at 4° C.). Finally, the oxidized protein fraction was dialyzed into 100 mM ammonium bicarbonate and digested with Tp as described above.

Jurkat T-cells (clone E6-1, ATCC TIB-152) were grown and harvested as described in the art (see, for example, Brill et al (2004) Anal. Chem. 76:2763-2772). The cellular protein was isolated as described above and subsequently digested overnight at 37° C. with sequencing-grade modified trypsin (Tp) (Promega, Madison, Wis.) using a substrate/enzyme ratio of 50:1 (w/w) in a solution of 100 mM ammonium bicarbonate, 4 M urea, pH 8.0. The proteolytic digest was preparatively desalted on a C18 reversed-phase cartridge (Haisil, Higgins Analytical, Mountain View, Calif.), and taken to dryness in a speed vac.

A performic acid oxidation solution was prepared by mixing 1 mL 88% formic acid with 100 μL 30% H₂O₂ that was first allowed to sit at RT for 2 h. 300 μL of this solution was then added to the dried tryptic peptides (7.5 mg), and the oxidation proceeded at RT for 1 h, followed by subsequent evaporation in vacuo. Finally, the tryptic peptides were reconstituted in 500 μL 0.1% acetic acid (v/v) and preparatively fractionated on a C18 reversed-phase cartridge into 5, 15, 25, and 40% acetonitrile-0.1% acetic acid (v/v) fractions successively. Alkaline β-elimination and Michael addition using a fluorous labeling reagent can then be performed as described herein.

### Example 8

### Derivitization and Enrichment of Oxosteroids From Human Plasma

Neutral steroid compounds, such as testosterone, androsterone and progesterone perform a number of metabolic roles, including stimulation of skeletal muscle growth and maintenance of reproductive and related tissues. The present invention provides methods and compositions for fluorous labeling of oxosteroids having a free (i.e., nonconjugated) ketone moiety, thereby providing a mechanism for partial purification of the compounds from a plurality of metabolites in a biologically-derived sample. In addition to reducing the complexity of the metabolomic sample to be analyzed, reaction of the steroid ketone moiety with, for example, an aminooxy-type fluorous labeling reagent, provides labeled molecules that often have a greater positive electrospray ionization efficiency than the starting metabolite, potentially enhancing the detection characteristics of the labeled species (and improving the sensitivity) during analysis by mass spectrometry.

Oxosteroidal compounds, as well as other relatively hydrophobic metabolites containing ketone or aldehyde groups, can be fluorous labeled as follows. A biological sample (in the case, human plasma) is added to an equal volume of acetonitrile, vortex-mixed for approximately 30 seconds, and centrifuged at 1500 g for 10 minutes at 4° C. The supernatant is removed, diluted with water (e.g., by 10-fold), and loaded onto a preconditioned bed of Oasis™ HLB solid phase extraction resin (Waters Corporation, Milford Mass.). The loaded resin is washed with three bed equivalents each of water and methanol:water (70:30), and the retained components are eluted with three bed volumes of ethyl acetate. The solvent is evaporated, and the residue is dissolved in approximately 50 μL of methanol.

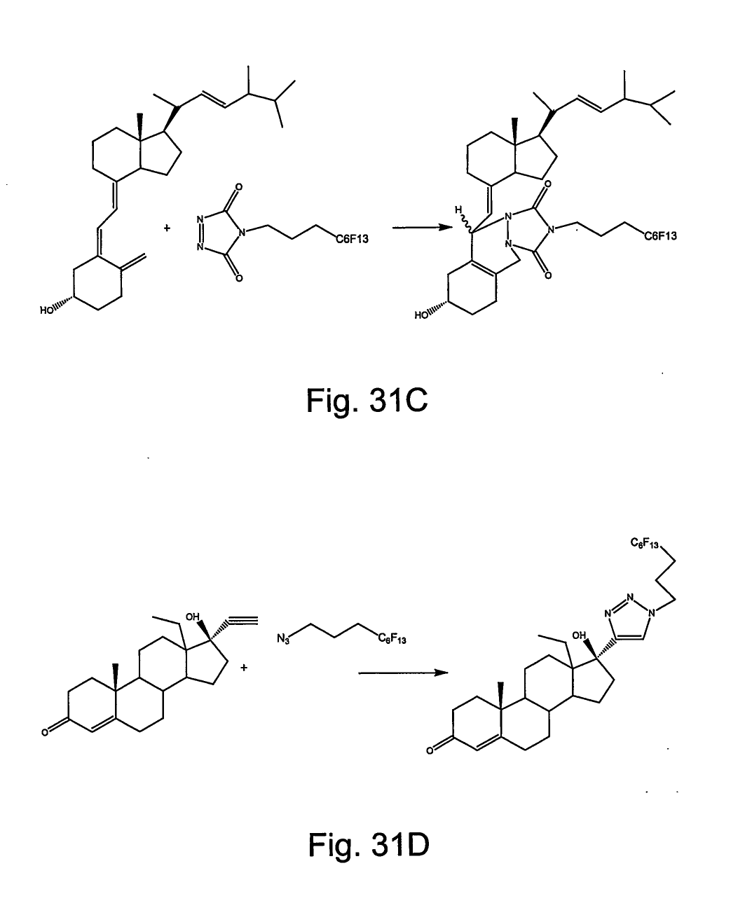

A small quantity (e.g., 5 μg) of an aminooxy-type fluorous labeling reagent (such as compounds 10 or 14, see Table 2) dissolved in 50 μL of methanol containing 0.5 mg of trichloroacetic acid is added to the eluted metabolites, and the mixture is kept at 50° C. for two hours. The reaction mixture is cooled and diluted with an equal volume of water. FIG. 31A provides an exemplary fluorous labeling reaction, as demonstrated for the neutral steroid testosterone and fluorous labeling reagent 14.

Optionally, any excess fluorous reagent is removed by treating the reaction mixture with a 4-benzyloxybenzaldehyde polystyrene resin (Novabiochem, Darmstadt, Germany). The fluorous-labeled sample components are separated from non-labeled species using a fluorous separation composition, such as a fluorous solid phase extraction cartridge. After loading the cartridge, the labeled metabolites are thoroughly rinsed with methanol:water (80:20), and the fluorous-derivatized species are eluted in 100% methanol. Alternatively, the diluted reaction mixture can be directly loaded onto a fluorous HPLC column, thoroughly rinsed with methanol:water (80:20), and subjected to direct LC/MS ESI analysis (e.g., using a shallow elution gradient of 80 to 100% methanol).

Other relatively hydrophobic metabolites containing ketone or aldehyde groups (for example, indole alkaloids) can also be labeled and analyzed in a similar manner. See, for example, Liu S. et al. “Use of oxime derivatives to enhance ionization of neutral ketone-containing species (metabolite analysis)” (2000) Rapid Comm Mass Spec 14:390 and Lemieux et al. (1998) Trends in Biotech 16:506.

### Example 9

### Derivitization and Enrichment of 1,2 Diol-Containing Steroids from Rat Brain

A similar reaction scheme can be designed for fluorous labeling of metabolite species possessing vicinal diols, using any of a number of known chemistries that target adjacent hydroxyl moieties. For example, boronic acid-containing reagents such as those described by Higashi et al. (2002 Analytical Sci. 18:1301-1307) can be substituted or further modified with fluorous moieties, to provide fluorous labeling reagents for use in the present invention. In one exemplary embodiment, fluorous boronic acid-containing reagent 18 is used to derivatize 4-hydroxy estradiol (see FIG. 31B).

Although this procedure can be used to label various classes of 1-2-diol containing species in a metabolomic sample, the most abundant metabolites that bear this functionality (i.e. sugars) can optionally be removed from the sample prior to performing the labeling reaction, e.g., by a solid phase extraction step using an Oasis™ cartridge.

A tissue sample, such as whole rat brain, is homogenized in methanol: acetic acid (100:1) using an ultrasonic homogenizer, and the concentration of the homogenate is adjusted to 100 mg tissue/mL solution. Approximately 0.5 mL of the homogenate is centrifuged at 1500 g for 10 min at 4° C., after which the resulting supernatant is removed and diluted with 2 mL of water. This solution is loaded onto a preconditioned bed of Oasis HLB solid phase extraction resin. The loaded resin is washed with three bed equivalents each of water and methanol:water (70:30), and the retained components are eluted with three bed volumes of ethyl acetate.

The solvent is evaporated, and the residue is dissolved in 50 μL of pyridine containing 0.5 mg of the fluorous boronic acid 18. After reaction for one hour at 50° C., the solvent is evaporated, and the residue is dissolved in a 50:50 solution of methanol:water. Optionally, any excess fluorous reagent can be removed by first incubating the mixture with 1-glycerol polystyrene resin (Product # 01-64-0408 Novabiochem) in a manner similar to that known in the art for removing excess acrylate reagent with a thiol-bearing resin.

This mixture is loaded onto a fluorous solid phase extraction cartridge, thoroughly rinsed with methanol:water (80:20), and the fluorous-derivatized species are eluted in 100% methanol. Alternatively, the diluted reaction mixture is directly loaded onto a fluorous HPLC column, thoroughly rinsed with methanol:water (80:20) and subjected to direct LC/MS ESI analysis running a shallow gradient from 80 to 100% methanol.

Alternatively, periodate oxidation of a metabolite of interest can be used to generate two aldehyde groups that can be ligated with the fluorous aminooxy moiety, such as previously described herein for carbohydrate-containing sample members. Optionally, the duo-labeled metabolite species can be separated from singly-labeled species prior to (or during) analysis.

### Example 10

### Selective Reaction and Isolation of Cis-Diene-Containing Molecules with Fluorous-Modified Cookson-Type Reagents

In a similar manner, molecules containing cis-diene moieties, such as vitamin D, can be labeled using Cookson-type reagents (e.g., maleimides) which have been substituted or otherwise coupled to a fluorous label. As an exemplary embodiment, a fluorous Cookson-type reagent 13 is used to derivatize the cis-diene containing molecule Vitamin D2 (see FIG. 31C). As also seen for the labeling of neutral steroid species with aminooxy-type fluorous labeling reagents, addition of the fluorous label has the added benefit of altering the electrospray ionization efficiency (e.g., providing a greater positive ES ionization efficiency than the unlabeled metabolite, assuming that the labeling reagent does not possess charge-bearing capability). See, for example, Yeung et al. “Cookson-type derivatization to enhance MS efficiency” (1995) Biochem. Pharmacol. 49:1099; and Werner et al. “Use of fluorous dienophiles as scavengers” (2003) Org. Letters 5:3293.

### Example 11

### Selective Reaction and Isolation of Terminal Alkyne-Containing Molecules With Azide-Type Fluorous Labeling Reagent

Huisgen 1,3-dipolar cycloaddition-type ligation reactions, sometimes referred to as “click chemistry” ligations, can also be used to target biologically-derived sample components having (or which have been modified to incorporate) a terminal alkyne moiety. In these reactions, an azide-type fluorous labeling reagent reacts with the alkyne-containing target species in the sample in what is typically an exerogenic process to form a triazole (see, for example, Rostovtsev et al. (2002) Angew Chem Int Ed 41:2596-2599 and references cited therein). An added advantage is the stability of the azide reagents under aqueous as well as organic reaction conditions, thus reducing the need to generate or prepare the biologically-derived sample in a non-aqueous environment.

FIG. 31D depicts a reaction scheme for modification of the steroid norgestrel. As noted by Rostovtsev et al., supra, 1,4-versus 1,5-regioselectivity of the product can be controlled in part through the selection of the copper catalyst. Exemplary fluorous azides for use in targeting of alkyne-containing components in a biologically-derived sample include azide compositions 15a and 15b. As with some previously-described labeling reactions, addition of the fluorous label to the sample member has the added benefit of producing a modified sample component having a greater positive electrospray ionization efficiency than the starting metabolite.

### Example 12

### Selective Reaction and Isolation of Primary Amine-Containing Molecules

Biologically-derived sample members containing primary amines, as opposed to secondary, tertiary and quaternary amines, can be selectively targeted for fluorous labeling using a thiol-type fluorous labeling reagent and o-phthaldehyde (FIG. 32A). The reaction of primary amines with o-phthaldehyde in the presence of thiols is a well-known reaction used, for example, for derivatization of polypeptide lysates to produce fluorescent derivatives (see, for example, Jones and Gilligan (1983) J. Chromatogr. 266:471-482).

### Example 13

### Selective Reaction and Isolation of Free Thiol-Containing Sample Members

Biologically-derived sample members containing a free thiol moiety can be fluorously labeled using, for example, any of a number of maleimide-type fluorous labeling reagents described herein. FIG. 32B depicts an exemplary reaction in which the metabolite homocysteine is reacted with fluorous labeling reagent 2b.

### Example 14

### Differential Labeling and Quantitation

An exemplary pair of isotopic reagents includes, but is not limited to, tridecafluorooctyl acrylate (compound 4a) and its 3,4,5,6,7,8-¹³C₆ tridecefluorooctyl analog. Protein samples (1 mg) to be compared are reduced with TCEP and digested with trypsin. The digests are desalted, dried, and reconstituted in 200 μL dimethyl formamide (DMF), and 2.5 μL 100 mM sodium carbonate, pH 8.0. 1 μL of tridecafluorooctyl acrylate 4a or the ¹³C₆ analog are added to each sample individually, and the reactions are allowed to proceed overnight at room temperature. The samples are combined, and unreacted acrylates are removed from the mixture by incubation with 4 mg N-2-mercaptoethylaminomethyl polystyrene beads (NovaBiochem) at room temperature for 2 hours. Fluorous solid phase extraction (FSPE) is performed as described in Example 1, to isolate the fluorous labeled (and thus cysteine-containing) peptides, each of which exists as an isotopic pair separated by 6 Daltons per cysteine moiety. The relative concentration between the two samples is reflected in the pair's signal intensities.

In addition to ¹³C substituted reagents, deuterium, ¹⁸O and/or ¹⁵N analogs of fluorous labeling reagents can also utilized in the methods of the present invention (see, for example, compound 6b). As an added benefit, the differentially labeled sample components typically have similar ionization properties and show minimal changes in the reversed-phase retention times (except in the case of ²H labeling).

### Example 15

### Crosslinking Reagents for 3D Structural Studies

The present invention also provides methods for determining the relative three-dimensional orientation (e.g., 3D mapping) of two or more chemical moieties either within the same protein, or between different proteins that exist as part of a protein complex. In this aspect, either hetero- or homo-multifunctional fluorous labeling reagents include the appropriate chemically-reactive functional groups needed to selectively react with two specified. chemical moieties (amino acid functionalities) in the protein. The two specified chemical functionalities being targeted for crosslinking with the fluorous labeling reagents should exist within a distance equal to or less than that spanned by the chemically-reactive functional groups in the fluorous labeling reagents. The fluorous moiety of the hetero- or homo-multifunctional fluorous labeling reagent is then used to selectively isolate these crosslinked species.

An example of such fluorous crosslinking reagents includes, but is not limited to, the homofunctional reagent bis(sulfosuccinimidyl)-2H,2H,3H,3H,10H,10H,11H,11H-perfluorododecanedionate 11. This fluorous labeling reagent selectively reacts with primary amines (i.e. lysine residues), and can effectively form crosslinks between any two such functionalities that are positioned less than approximately 12 carbon chain lengths apart (e.g., the length of the linkers and fluorous moiety). In an exemplary embodiment, purified protein complexes dissolved in Na2HPO₄, pH 8.0 are added to an excess of the fluorous crosslinker reagent dissolved in DMF, and the reaction is allowed to proceed at room temperature for approximately 30 minutes. The reaction mixture is then digested, and the contents are subjected to FSPE. Peptides containing the fluorous tag are separated from non-labeled species and subjected to mass spectrometry studies to determine the sites of crosslinking.

In a related aspect, the bifunctional fluorous labeling reagents of the present invention can optionally be used for purposes other than crosslinking of (identical or different) sample member functional groups. For example, fluorous labeling reagents having a carboxylic acid moiety positioned directly adjacent to fluoroalkyl chain, as well as a second chemically-reactive functional group shielded from the inductive effect of the fluorine atoms, would have potential use, e.g., in providing a charged moiety for analysis by tandem MS. The carboxylic acid would be totally deprotonated, thus providing a negative charge to the fluorous labeled sample components. An exemplary embodiment of a carboxylate-containing fluorous labeling reagent is provided as compound 5e.

As another example, a fluorous moiety coupled to the lysine-specific labeling reagents described in International PCT publication WO 03/056299 to Peters et al., could be used to produce a highly basic, but not permanently charged, amine-targeted fluorous labeled sample components that would, as an added benefit, also exhibit an increased ionization efficiency.

### Example 16

### Multiplexing of Analyses

In yet another embodiment using the compounds and derivatives disclosed herein, the present invention provides methods for the simultaneous analysis of multiple samples. In this aspect, a series of reagents having the same chemically-reactive functional group but different fluorous moieties are used to individually label a series of samples, such that each sample is reacted with a different fluorous tag. The resulting samples are pooled, and the fluorous labeled species are separated from non-tagged species using FSPE. The retained species are then batch eluted and analyzed simultaneously (i.e., by MALDI TOF MS), with the difference in masses between analytes indicating the nature of the tag and thus the identity of the sample from which it arose, while the relative intensities of the tagged species is proportional to their respective concentrations. Alternatively, the pooled, retained samples are subject to fluorous chromatography such that the tagged samples elute from the column in an order proportional to their fluorine content. Additionally, different tags be used exclusively with different reactions conditions such that a given peptide can have several tags of different lengths that indicate what combination of amino acid functionalities and/or PTMs were present.

An example of such a multiplex analysis includes, but is not limited to, the discovery and relative assessment of serine/threonine phosphorylation of a given biologically-derived sample member. Three different samples of the targeted substrate (prepared, for example, under different conditions) are subjected to β-elimination reactions, and each is then individually labeled with 1H,1H,2H,2H-perfluorodecane-1-thiol (1a), 1H,1H,2H,2H-perfluorooctane-1-thiol (1b), or 1H,1H,2H,2H-perfluorohexane-1-thiol (1f).

The samples are combined, and subjected to FSPE such that all fluorous tagged species are retained. If the retained species are batch eluted and subjected to MALDI analysis, a series of three peaks differing in mass by 100 Daltons appears for each labeled peptide, and the mass of the peptide itself can easily be calculated. If subjected to fluorous chromatography, an individual analyte labeled with a C₄F₉ tag will elute from the column before the one labeled with C₆P₁₃, which will elute from the column before the one labeled with C₈F₁₇, thus providing individual windows for analysis.

### Example 17

### Alternative Approach to Fluorous Labeleing of Phosphorylated Species

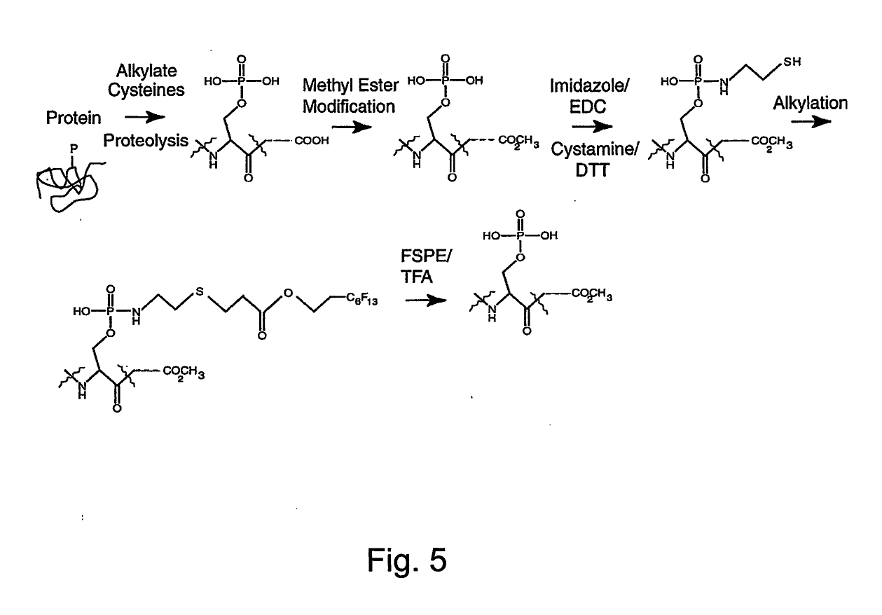

FIG. 5 depicts an exemplary multistep reaction scheme for fluorous labeling of phosphorylated peptides, similar to the methodology described by Zhou et al (2001) Nature Biotechnol. 19: 375-378. The reaction involves carboxylic acid methylation under acidic conditions, EDC-mediated coupling of cystamine to give a phosphoramidate, alkylation with a fluorous Michael acceptor, and acid release of the methylated phosphopeptides after FSPE, providing isolation and/or enrichment of phosphoserine, phosphothreonine and phosphotyrosine containing species.

Tryptic peptides were desalted using peptide macrotrap cartridges (Michrom Bioresources, Auburn, Calif.) and methylated according to Brill et al. (2004) Anal Chem. 76: 2763-2772. Dried, methylated peptides were reconstituted in 50 μL of 1 M imidazole. This solution was added to 4 mg of EDC (final concentration ˜0.5M), and the mixture incubated at room temperature for 2 hr. The mixture was loaded onto C18 columns (360/200 μm O.D/I.D. fused silica packed with 12 cm POROS 10R2), and washed with 20 μL of water. The column was then washed with the following: 1 M cystamine, pH 8.0 at 2 μL/min for 2 hr at 56° C., 20 μL water, 10 mM DTT for 1 hr at 50° C. at a flow rate of 3 μL/min and 20 μL water. Peptides were eluted from the column with 70% acetonitrile, and evaporated to dryness.

Dried peptides were reconstituted in 20 μL of 20 mM tridecafluorooctylacrylate in DMF, 0.75 μl 50 mM sodium carbonate pH 8 was added, and the mixture incubated for 2 hr at room temperature. Excess reagent was removed with the addition of 0.5 mg of N-2-mercaptoethylaminomethyl polystyrene beads (Novabiochem) and incubation at room temperature for 1 hr. The resulting peptide mixtures were diluted 5-fold with 60% MeOH containing 10 mM ammonium formate and enriched by FSPE as described. The retained, fluorous-tagged fraction was dried, and reconstituted in 95% TPA for 30 min to cleave the phosphoramidate bond. TFA was removed by vacuum centrifugation, and the now fluorous-free, methylated phosphopeptides reconstituted for MS analysis.

### Example 18

### Fluorous Labeling of Intact Protein

The following example demonstrates that intact protein can be fluorously labeled and readily handled in typical protein manipulations, such as 1D gel and in-gel digestions.

Bovine serum albumin (˜40 μM) was reduced with 10 mM TCEP in 6 M guanidinium hydrochloride, 20 mM Tris, pH 8.0 buffer for 10 minutes at room temperature, and reacted with 20 mM N-(1H, 1H,2H,2H-perfluorooctyl)iodoacetamide for 1 hour in the dark by addition of an equal volume of a THF solution of the fluorous iodoacetamide. Excess reagents were removed using a disposable gel filtration spin column packed with Biogel P6 beads (Micro Biospin P6, Bio-Rad, Hercules, Calif.). The desalted fluorous-labeled protein was recovered by collection of the appropriate filtrate fraction upon centrifugation.

The desalted fluorous-labeled protein fraction was dried briefly in a speed-vac to remove the tetrahydrofuran, and combined with an equal volume of gel loading buffer (100 mM Tris, pH 6.8, 50% glycerol, 0.1% bromophenol blue, 1% SDS). SDS-PAGE was performed using a 150 V constant voltage after loading several micrograms of derivatized protein into each well of a 12 well, 1 mm×8 cm×8 cm 10-20% Tris-Glycine polyacrylamide gel (Invitrogen, Carlsbad, Calif.). Following electrophoresis, the gel slab was stained with colloidal Coomassie blue (Invitrogen, Carlsbad, Calif.) for 4 hours, followed by destaining in water overnight.

In-gel trypsin digestion was performed as follows. Excised protein gel bands were cut into small cubes (2-3 mm) with a new razor blade and added to 0.5 mL microcentrifuge tubes. The gel cubes were subjected to several wash and dehydration cycles (ten minutes incubation with 50 μL of 100 mM ammonium bicarbonate, removal of the liquid, ten minutes incubation with 25 μL acetonitrile and removal of the liquid. The dehydrated gel cubes were vacuum dried for 5 minutes, rehydrated in 20 μL of 50 mM ammonium bicarbonate solution containing 10 ng/μL trypsin (Promega, Madison, Wis.) and incubated at 37° C. overnight. Tryptic peptides were recovered by repeated extraction (3×) with 80% acetonitrlile/0.2% TFA (v/v). The peptide extracts were combined and dried in vacuo. The samples were reconstituted in 60% methanol containing 10 mM ammonium formate as preparation for fluorous solid phase extraction (described elsewhere).

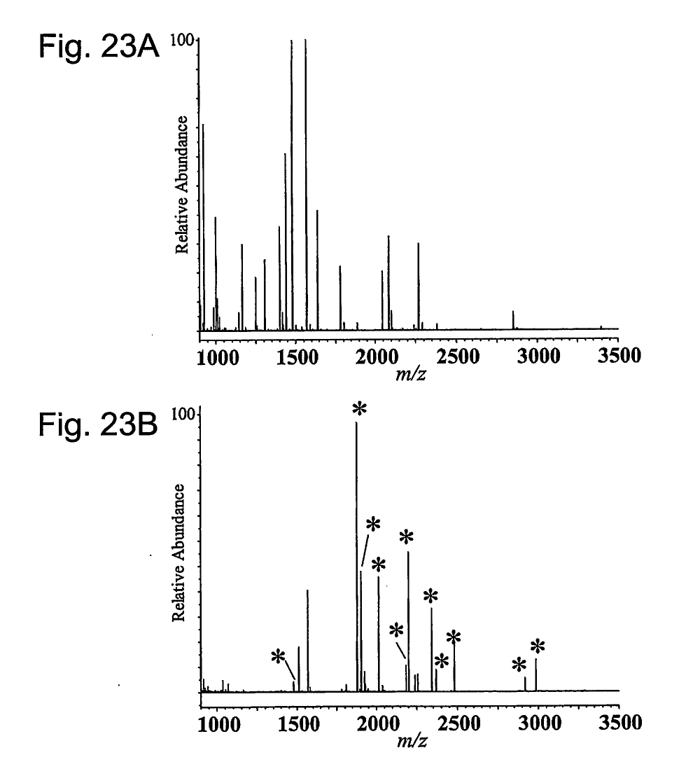

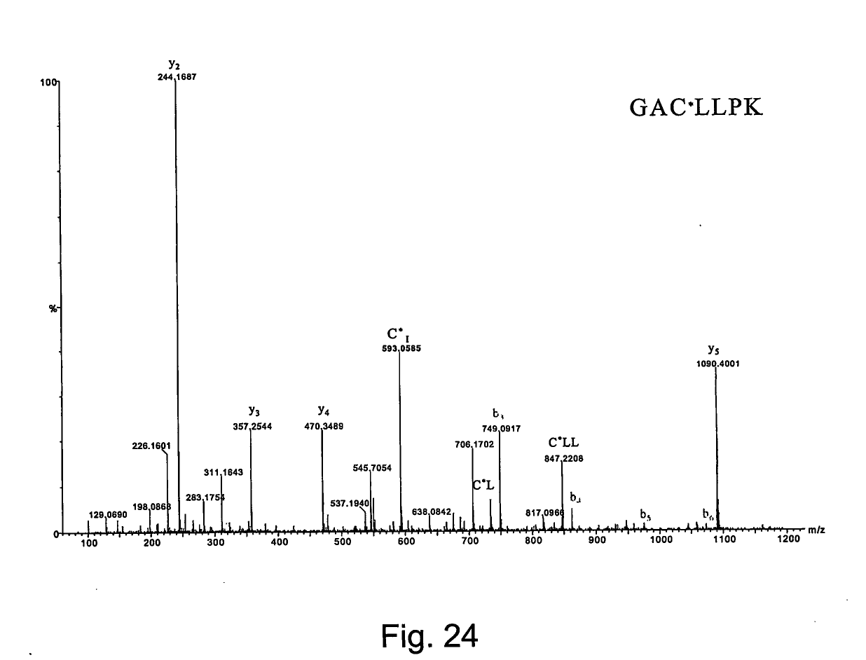

FIG. 23A provides the MALDI spectrum generated for the tryptic digest of BSA after reduction with TCEP and reaction with N-[(3-perfluorooctyl)-propyl]iodoacetamide as described above. The sample was then subjected to FSPE, and the peptides retained and subsequently eluted were subjected to MALDI MS (FIG. 23B). The peaks denoted with the “*” symbol are fluorous tagged cysteine-containing peptides. FIG. 24 depicts the tandem MS data for the 2+ charge state of fluorous labeled peptide GAC*LLPK (SEQ ID NO:35). The peak labeled C*₁ is the immonium ion of the modified cysteine residue.

### Example 19

### Cleavable Reagents

The fluorous labeling reagents of the invention also include embodiments in which the fluorous label can be cleaved or otherwise released from the associated biologically-derived sample component, e.g., to facilitate recovery of the biologically-derived component during an enrichment or isolation process.

An exemplary embodiment of a cleavable fluorous labeling reagent of the invention is compound 25, 6-[3-(3,3,4,4,5,5,6,6,6-nonafluoro-hexyldisulfanyl)-propionylamino]-hexanoic acid 2,5-dioxo-pyrrolidin-1-yl ester. Cleavable reagent 25 was synthesized by adding 50 mg LC-SPDP (succinimidyl 6[3-(2-pyridyldithio)-propionamido]hexanoate, 59 μmol, bought from Pierce, Rockford Ill.) to 32.9 mg of 1H,1H,2H,2H perfluorohexanethiol in 90% TBF/10% 50 mM Na₂HPO₄, pH 7.2. After 1 hour, solvent was removed under reduced pressure.

Peptide modification was performed as follows: 1 nmol bradykinin in 100 mM sodium acetate pH 7.7 (10 μL) was added to 110 nmol of fluorous labeling reagent 25 in 110 μL DMF. After 2 hr, unreacted label was removed by incubation with aminopropyl-functionalized polystyrene beads (Novabiochem) for 2 hr. The resulting isolated modified peptide was found to have a m/z of 1539.6 by MALDI TOF MS. The modified peptide was then incubated with 100 mM TCEP to cleave the disulfide bond. The resulting peptide was found to have a m/z of 1261.6 by MALDI TOP MS, indicating loss of the fluorous tag.

General Materials and Methods

Peptide Capture and Desalting

μC₁₈ ZiptipS™ (Millipore, Bedford, Mass.) were used to capture, concentrate and desalt peptides before labeling. Activation was performed by aspirating 5×10 μL aliquots of 80% Acetonitrile/0.1% trifluoroacetic acid (IfA) (v/v). Tips were equilibrated similarly by using 0.1% TFA (v/v). Peptide samples were prepared in 0.1-0.5% TFA (v/v) and loaded by repeated aspiration. Loading of fluorous-derivatized peptides was preferably performed with the addition of a minimum of 20-25% methanol in the loading solution to reduce precipitation. The tips were then washed with aliquots of 0.1% TPA (v/v), and peptides eluted by repeated aspiration in 4-5 μL aliquot of 80% Acetonitrile/0.1% TFA (v/v).

‘Fluorous’ HPLC

Fluoroflash, Tridecafluoro Silica (TDF) & Pentafluorophenyl Silica (PFP) (Silicycle, Montreal, QC, Canada) 180 Å pore size, 3 μm particles were pressure packed into fritted fused silica (360/200 or 360/100 1 m O.D./I.D.) to a length of ˜12 cm. Gradient elution was performed using an ammonium formate modified mobile phase, and interfaced via ESI to QqTOF mass spectrometer.

Mass Spectrometry and Data Analysis

MALDI-TOF MS was performed on a Bruker Biflex III in delayed extraction/reflector mode. Peptides were deposited on a MALDI target using the dried droplet method by first mixing a sample with a stock solution of 2,5-dihydroxybenzoic acid matrix (DHB, 10 mg/mL in 50% acetonitrile/0.2% trifluoroacetic acid v/v). Laser attenuation was set at 40-45 with several hundred shots averaged. Acceleration voltages were set to 19 kV (IS/1) & 15.2 kV (IS/2), with the reflectron voltage set at 18.7 kV.

Capillary LC-ESI MS and tandem MS were performed using a Monitor C18 packed capillary column (3 μm particles, 100 Å, 75 μm or 300 μm I.D., 8-15 cm length, available from Column Engineering Inc., Ontario, Calif.) interfaced to a hybrid quadrupole time-of-flight (QqTOF) mass spectrometer (Micromass Q-TOF 2, Waters, Milford Mass.) operating in survey scan mode. The 15 cm column was typically run at 3 μL/min using a gradient generated using 0.5M acetic acid (A) and acetonitrile with 0.5M acetic acid (B). ESI was performed using a spray voltage of 4 kV and cone voltage of 30V in collision gas (argon). Mass (m/z) range of 450-1800 was analyzed for intensity threshold MS/MS triggering (LM Res, HM Res=5 corresponding to ˜5Da isolation window). Collisional dissociation energies were automatically adjusted according to determined parent mass and charge state.

Data analysis from samples prepared by alkaline β-elimination/fluorous Michael addition and analyzed by capillary LC-MS/MS (cLC-MS/MS) were searched by including the possible conversions of cysteine to cysteic acid (residue mass=150.9939 Da exact mass, 151.1411 Da average mass), and methionine to methionine sulfone (residue mass=163.0303, 163.1949), as well as the possible C₆F₁₃ sulfoxide side-chain modifications to former pS (residue mass=465.0068, 465.2352) and pT residues (residue mass=479.0224, 479.2621). The latter modifications were identified based on both their unique residual masses as well as the presence of characteristic neutral loss fragmentation products, dehydroalanine (pS) or β-methyldehydroalanine (pT), in the tandem MS patterns. Fluorous labeled phosphopeptide derivatives that were allowed to oxidize to the β-linked fluorous sulfone exhibit much less side-chain tandem MS fragmentation, and were characterized by recognition of the intact residual mass and not by neutral loss product fragments.

cLC-MS/MS data analysis of the fluorous derivatives of cysteinyl peptides allowed for their identification by recognition of the unique residual masses formed by the respective reaction of the cysteine residue with either TDFOA (residue mass=521.0330, 521.2994) or the fluorous iodoacetamide (residue mass=620.0426, 620.3303).

The tandem MS spectra of iodoacetamide-derivatized peptides display a characteristic signal ion at m/z of 593 corresponding to the immonium ion of the derivatized cysteine residue. This signal does not dominate the spectra like some neutral loss species (i.e. pS/pT-containing peptides), but it is large enough in intensity and high enough in mass that its presence can serve as a ‘diagnostic’ signal even on ion traps operating under standard conditions. The acrylate functionalized species show a similar immonium ion signal.

Analysis of samples prepared by 8-amino “guanidination”/α-amino fluorous derivatization involved searching for the variable modifications of lysine depending on the reagent employed, fluorous acylation of N-termini (Δ mass 32 275.011, 275.0937) and in the case of polyubiqiatin, fluorous amidation of the glycine-glycine isopeptide-modified lysine side chain (residue mass=516.1419, 516.3639).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1-69. (canceled)

70. A fluorous labeling reagent comprising a fluorous moiety coupled to a bioconjugation agent comprising a chemically reactive functional group, wherein the fluorous labeling reagent comprises five or more fluorine atoms.

71. The fluorous labeling reagent of claim 70, wherein the fluorous moiety comprises five or more fluorine atoms coupled to contiguous carbon atoms.

72. The fluorous labeling reagent of claim 70, wherein the fluorous moiety comprises two or more clusters of carbon-coupled fluorine atoms separated by non-fluorous linker regions.

73. The fluorous labeling reagent of claim 70, wherein the labeling reagent comprises a first fluorous moiety coupled at a first position on the bioconjugation agent and a second fluorous moiety coupled at a second position on the bioconjugation agent.

74. The fluorous labeling reagent of claim 70, wherein the labeling reagent comprises an aqueous compatible reagent.

75. The fluorous labeling reagent of claim 70, wherein the fluorous moiety comprises one or more ¹³C atoms, ¹⁵N atoms, ¹⁸O atoms, or deuterium atoms.

76. The fluorous labeling reagent of claim 70, wherein the chemically-reactive functional group comprises an isotopic label.

77. The fluorous labeling reagent of claim 70, wherein the fluorous labeling reagent further comprises a non-fluorous linker element positioned between the bioconjugation agent and one or more of the fluorine moieties.

78. The fluorous labeling reagent of claim 77, wherein the linker element comprises an alkyl chain between two and twenty carbons in length.

79. The fluorous labeling reagent of claim 77, wherein the linker element comprises one or more ¹³C atoms, ¹⁵N atoms, ¹⁸O atoms, or deuterium atoms.

80. The fluorous labeling reagent of claim 77, wherein the linker element comprises a releasable element.

81. The fluorous labeling reagent of claim 80, wherein the releasable element comprises a peptide cleavage site.

82. The fluorous labeling reagent of claim 80, wherein the releasable element comprises a disulfide bond.

83. The fluorous labeling reagent of claim 82, wherein the reagent is:

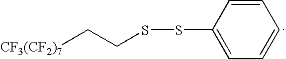

84. The fluorous labeling reagent of claim 82, wherein the reagent is:

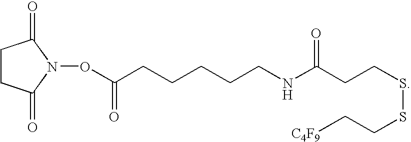

85. The fluorous labeling reagent of claim 70, wherein the chemically-reactive functional group comprises a maleimide, a halogen β-ketone, a disulfide exchange reagent, a phenylglyoxal derivative, an anhydride, an acrylate, an NHS ester, a thiol, a dialkyl pyrocarbonate, an aminooxy group, or a hydrazine.

86. The fluorous labeling reagent of claim 70, wherein the chemically-reactive functional group comprises an isotopic label.

87. The fluorous labeling reagent of claim 70, wherein the fluorous labeling reagent comprises an fluoroalkyl thiol, a fluorous alkoxyamine, a fluorous NHS ester, a fluorous sulfo-NHS ester, or a fluorous azide.

88. The fluorous labeling reagent of claim 70, wherein the fluorous labeling reagent further comprises a second bioconjugation agent.

89. The fluorous labeling reagent of claim 88, wherein the fluorous labeling reagent comprises first and second bioconjugation agents for derivatizing first and second amino acid-associated functional groups, and wherein the first and second amino acid-associated functional groups are similar functional groups.

90. The fluorous labeling reagent of claim 89, wherein the fluorous labeling reagent is

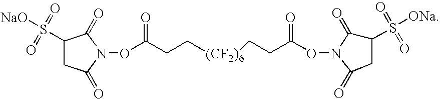

91. The fluorous labeling reagent of claim 88, wherein the fluorous labeling reagent comprises first and second bioconjugation agents for derivatizing first and second amino acid-associated functional groups, wherein the first and second amino acid-associated functional groups are differing functional groups.

92. The fluorous labeling reagent of claim 70, wherein the fluorous labeling reagent is inert under standard ionization and/or fragmentation conditions for mass spectroscopy.

93. The fluorous labeling reagent of claim 70, wherein the fluorous labeling reagent is selected from the group consisting of:
CF₂H(CF₂)₅CH₂CH₂SH,
(CF₃CF₂)₂CF(CF₂)₂CH₂CH₂SH,
(CF₃CF₂)₂CH(CF₂)₂CH₂CH₂SH,

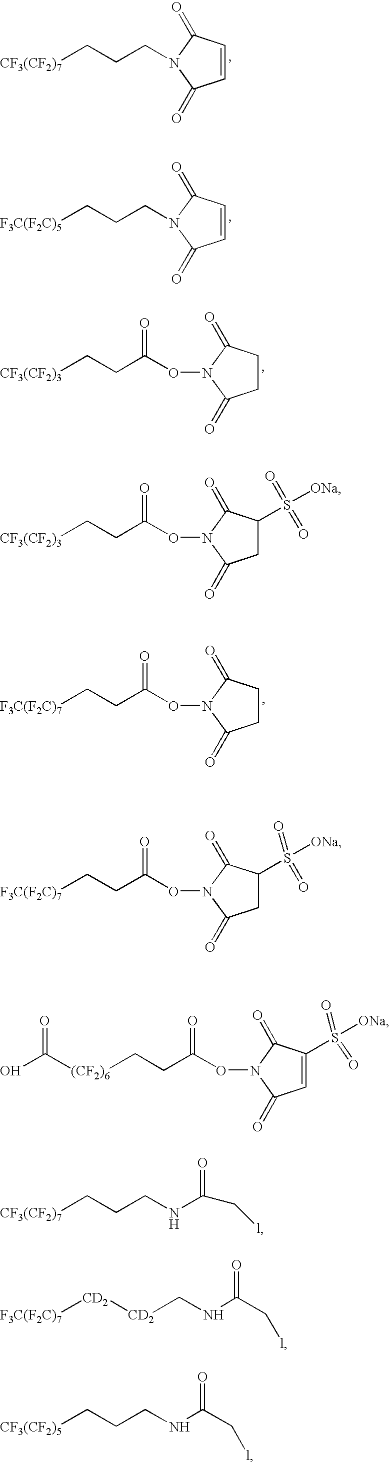

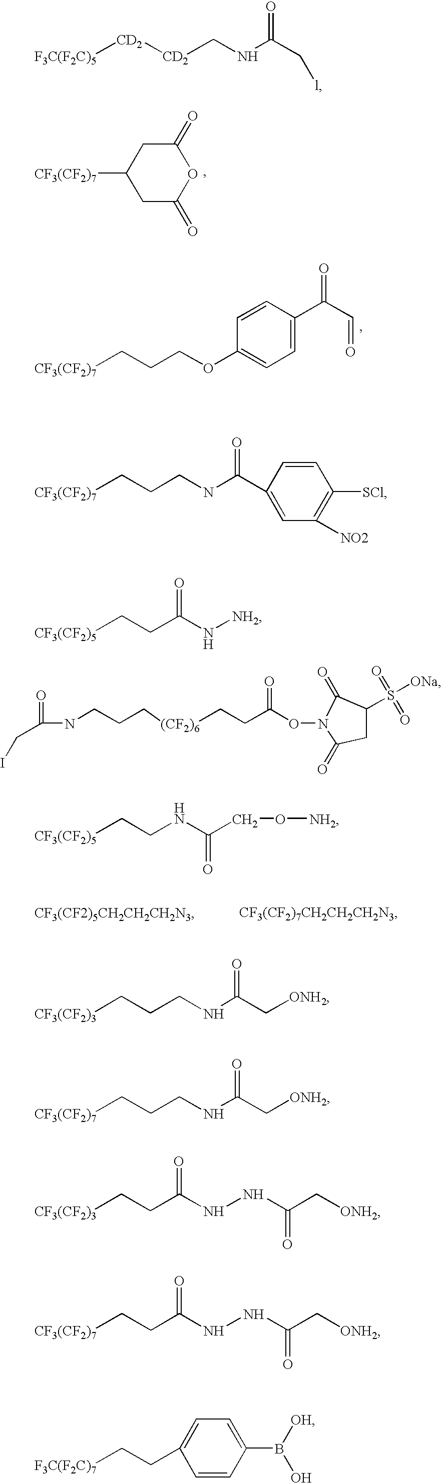

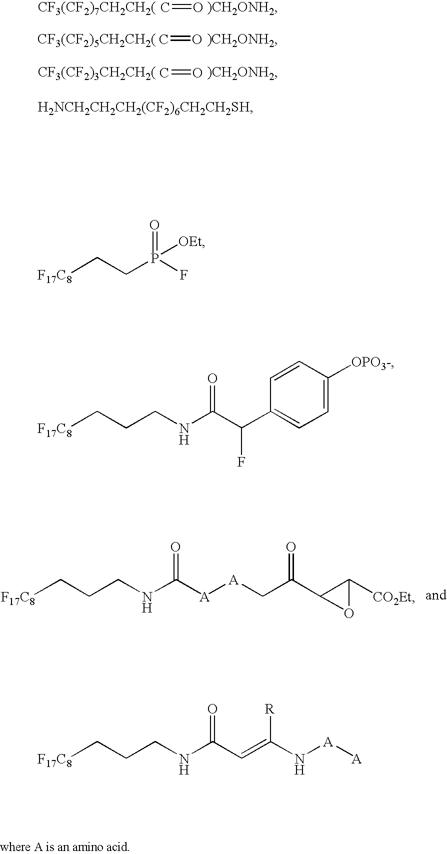

94. A set of fluorous labeling reagents for differential quantification of a biologically-derived sample, the set comprising two or more fluorous labeling reagents of claim 70, wherein the fluorous labeling reagents are differentially labeled with one or more stable isotopes.

95. The set of fluorous labeling reagents of claim 94, wherein the stable isotope comprises deuterium.

96. The set of fluorous labeling reagents of claim 94, wherein the stable isotope comprises ¹³C.

97. The set of fluorous labeling reagents of claim 94, wherein the stable isotope comprises ¹⁵N.

98. The set of fluorous labeling reagents of claim 94, wherein the stable isotope comprises ¹⁸O.

99-101. (canceled)
